(19) United States
(12) Patent Application Publication
(10) Pub. No.: US 2026/0116933 A1
(43) Pub. Date: Apr. 30, 2026

(54) INHIBITORS OF NFAT AND NFKB SIGNALING FOR IMPROVING IMMUNE CELL FUNCTION

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Scott JAMES, New York, NY (US); Jacob S. FISCHMAN, New York, NY (US); Alexander P. BOARDMAN, New York, NY (US); Marcel VAN DEN BRINK, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/258,139

(22) Filed: Jul. 2, 2025

Related U.S. Application Data
(63) Continuation of application No. PCT/US2024/010515, filed on Jan. 5, 2024.
(60) Provisional application No. 63/478,664, filed on Jan. 5, 2023.

(51) Int. Cl. C07K 14/47; A61K 35/17; A61K 40/11; A61K 40/31; A61P 35/02; C07K 7/08; C12N 5/0783; C12N 9/10; C12N 15/86

(52) U.S. Cl. CPC C07K 14/4703; A61K 35/17; A61K 40/11; A61K 40/31; A61P 35/02; C07K 7/08; C07K 14/4728; C12N 5/0636; C12N 9/1088; C12N 15/86; A61K 2239/48; C07K 2319/60; C12N 2510/00; C12N 2740/10043; C12Y 205/01018

(57) ABSTRACT

The present disclosure is directed to systems for inhibiting NFAT and/or NFkB signaling to improve the function of CAR expressing immune cells, e.g., CAR T cells. The system enables generation of immune cells engineered to express a CAR or multiple combinations of CARs (multi-CAR) and inhibitors of NFAT and/or NFkB signaling.

### CROSS-REFERENCE TO RELATED APPLICATIONS

This application is a Continuation of International Patent Application No. PCT/US2024/010515 filed Jan. 5, 2024, which claims priority to U.S. Provisional Patent Application No. 63/478,664, filed on Jan. 5, 2023, the contents of each of which are incorporated by reference in their entirety, and to each of which priority is claimed.

### SEQUENCE LISTING

The specification further incorporates by reference the Sequence Listing submitted herewith via EFS on Jul. 2, 2025. Pursuant to 37 C.F.R. § 1.52(e)(5), the Sequence Listing XML file, identified as 0727341773_ST26.xml, is 147,699 bytes and was created on Jul. 1, 2025. The Sequence Listing, electronically filed herewith, does not extend beyond the scope of the specification and thus does not contain new matter.

### BACKGROUND OF THE INVENTION

Chimeric antigen receptor (CAR) T cell therapy has demonstrated remarkable therapeutic activity in refractory B cell malignancies and myeloma. However, CAR T cells targeting both hematologic malignancies and solid tumors face a number of challenges that limit their safety and efficacy including antigen-loss or antigen-low escape of malignant cells; T cell exhaustion related to tonic CAR signaling and repetitive antigen-stimulation; immunosuppressive tumor microenvironments; lack of target antigen specificity for tumor cells; and CAR T cell-mediated cytokine release syndrome (CRS) and neurotoxicity. Synthetic biology approaches have produced multiple solutions to address these problems individually. For example, engineered co-expression of multiple CARs can overcome escape of antigen-low/negative disease. In addition, temporal manipulation of CAR expression or activation, regulation of CAR expression density, attenuation of CAR CD3ζ signal strength, and 4-1BB costimulation can attenuate tonic-signaling and antigen-stimulation-induced T cell dysfunction. Dominant negative and switch receptors blocking PD-1, CD200R1, and Fas can also improve CAR T cell activity in response to tumor-mediated immune suppression.

As tumor cells utilize many immune evasion strategies, CAR T cells may fail due to both exhaustion and tumor-mediated suppression. A combination of multiple synthetic biology solutions may be required to enhance CAR T cell activity. There are, however, challenges to coalescing multiple strategies due to limitations in packaging and delivery of large vector inserts encoding multiple transgenes. There is, therefore, a need in the art for systems and methodologies in disease treatment that improve the efficacy of T cells and CAR T cells.

### SUMMARY OF THE INVENTION

The presently disclosed subject matter provides a system is directed, in certain embodiments, to leucine zipper-based sorting systems adapted to facilitate the expression and coordination of inhibitor peptide sequences capable of improving the function of CAR expressing immune cells. In certain embodiments, the systems facilitate the generation of immune cells engineered to express multiple combinations of CARs (multi-CAR), safety-switches, switch receptors, and/or cytokines. For example, but not by way of limitation, the present disclosure is directed to systems comprising a plurality of nucleic acid constructs, wherein the plurality comprises:
(a) a first nucleic acid construct encoding a membrane bound polypeptide comprising:
   (i) an extracellular domain comprising a first leucine zipper sequence;
   (ii) a transmembrane domain; and
   (iii) an intracellular domain;
(b) a second nucleic acid construct encoding a soluble polypeptide comprising a second leucine zipper sequence capable of heterodimerizing with the first leucine zipper sequence and a signal peptide sequence;
   wherein each of the first and/or second nucleic acid constructs further encode one or more CAR, safety switch, switch receptor, and/or cytokine; and wherein the first or second nucleic acid construct further comprises a nucleic acid sequence encoding: (1) a nuclear factor of activated T cells (NFAT) inhibitor peptide or NFkB inhibitor peptide; and (2) a carrier protein (CP). In certain embodiments, the inhibitor peptide inhibits calcineurin signaling. In certain embodiments, the inhibitor peptide is a NFAT inhibitor peptide (NFAT inhibitor). In certain embodiments, the NFAT inhibitor comprises a PXIXIT calcineurin binding sequence, wherein X is any amino acid. In certain embodiments, the NFAT inhibitor is a VIVIT peptide or a variant thereof. In certain embodiments, the NFAT inhibitor comprises the amino acid sequence set forth in any one of SEQ ID Nos: 24-29. In certain embodiments, the inhibitor peptide comprises a sequence derived from the autoinhibitory domain of calcineurin. In certain embodiments, the inhibitor peptide comprises the amino acid sequence set forth in SEQ ID No: 30. In certain embodiments, the inhibitor peptide inhibits NFkB signaling. In certain embodiments, the inhibitor peptide is an NFkB inhibitor, or a degradation-resistant mutant thereof. In certain embodiments, the inhibitory peptide is a mutated NFkB inhibitor alpha (NFKBIAm). In certain embodiments, the NFKBIAm comprises the amino acid sequence set forth in SEQ ID No: 32.

In certain embodiments of the presently disclosed subject matter, the CP is selected from the group consisting of glutathione-S-transferase (GSTA1), enhanced green fluorescence protein (EGFP), and blue fluorescent (BFP). In certain embodiments, the nucleic acid sequence encoding the CP comprises a regulatable gene element. In certain embodiments, the regulatable gene element encodes a regulator motif that regulates expression of the inhibitory peptide. In certain embodiments, the regulator motif comprises a drug-stabilized signaling domain. In certain embodiments, the drug-destabilized signaling domain has a dihydrofolate reductase destabilization domain (DHFR-DD). In certain embodiments, the DHFR-DD comprises the amino acid sequence set forth in SEQ ID NO: 43. In certain embodiments, the drug is trimethoprim, analogs thereof, or derivatives thereof. In certain embodiments, the drug-destabilized signaling domain has a FK506 binding protein destabilization domain (FKBP-DD). In certain embodiments, the drug is Shield-1 (Shld1), analogs thereof, or derivatives thereof.

In certain embodiments of the presently disclosed subject matter, the first nucleic acid construct comprises the amino acid sequence set forth in SEQ ID NO: 34. In certain embodiments, the second nucleic acid construct comprises the amino acid sequence set forth in SEQ ID NO: 35. In certain embodiments, the nucleic acid sequence of the first nucleic acid construct further encodes a hinge domain. In certain embodiments, the hinge domain comprises the amino acid sequence set forth in any one of SEQ ID NOs: 52, 54 or 62.

In certain embodiments of the presently disclosed subject matter, the first and/or second nucleic acid construct comprises a promoter element that regulates expression of the inhibitor peptide. In certain embodiments, the promoter element is a NFAT responsive promoter element or an NFkB responsive promoter element. In certain embodiments, the NFAT responsive promoter element comprises the sequence set forth in SEQ ID No: 48. In certain embodiments, the NFkB responsive promoter element comprises the sequence set forth in SEQ ID No: 49. In certain embodiments, the inhibitory peptide comprises the amino acid sequence set forth in any one of SEQ ID NOs: 70-93.

In certain embodiments, the presently disclosed subject matter is directed to engineered immune cells comprising a system as described herein. In certain embodiments, the immune cell is a T cell. In certain embodiments, the immune cell is a CAR T cell.

In certain embodiments, the presently disclosed subject matter is directed to methods of modifying a cell comprising delivering to the cell, a system as disclosed herein. In certain embodiments, the cell is a mammalian cell. In certain embodiments, the mammalian cell is an immune cell. In certain embodiments, the immune cell is a T cell. In certain embodiments, the immune cell expresses at least one chimeric antigen receptor (CAR).

In certain embodiments, the presently disclosed subject matter is directed to engineered immune cells comprising:
(a) a first nucleic acid construct comprising a nucleic acid sequence encoding a CAR; and
(b) a second nucleic acid construct comprising a nucleic acid sequence encoding: (1) a nuclear factor of activated T cells (NFAT) inhibitor peptide or NFkB inhibitor peptide; and a (2) a carrier protein.
   In certain embodiments, the inhibitor peptide inhibits calcineurin signaling. In certain embodiments, the inhibitor peptide is a NFAT inhibitor. In certain embodiments, the NFAT inhibitor comprises a PXIXIT calcineurin binding sequence, wherein X is any amino acid. In certain embodiments, the NFAT inhibitor is a VIVIT peptide or a variant thereof. In certain embodiments, the NFAT inhibitor comprises the amino acid sequence set forth in any one of SEQ ID Nos: 24-29. In certain embodiments, the inhibitor peptide comprises a sequence derived from the autoinhibitory domain of calcineurin. In certain embodiments, the inhibitor peptide comprises the amino acid sequence set forth in SEQ ID No: 30. In certain embodiments, the inhibitor peptide inhibits NFkB signaling. In certain embodiments, the inhibitor peptide is an NFkB inhibitor, or a degradation-resistant mutant thereof. In certain embodiments, the inhibitory peptide is NFKBIAm. In certain embodiments, the NFKBIAm comprises the amino acid sequence set forth in SEQ ID No: 32.

In certain embodiments, the presently disclosed subject matter is directed to engineered immune cells comprising:
(a) a first nucleic acid construct comprising a nucleic acid sequence encoding a CAR; and
(b) a second nucleic acid construct comprising a nucleic acid sequence encoding: (1) a nuclear factor of activated T cells (NFAT) inhibitor peptide or NFkB inhibitor peptide; and a (2) a carrier protein (CP); and
   wherein the CP is selected from the group consisting of glutathione-S-transferase (GSTA1), enhanced green fluorescence protein (EGFP), and blue fluorescent (BFP). In certain embodiments, the nucleic acid sequence encoding the CP comprises a regulatable gene element. In certain embodiments, the regulatable gene element encodes a regulator motif that regulates expression of the inhibitory peptide. In certain embodiments, the regulator motif comprises a drug-stabilized signaling domain. In certain embodiments, the drug-destabilized signaling domain has a dihydrofolate reductase destabilization domain (DHFR-DD). In certain embodiments, the DHFR-DD comprises the amino acid sequence set forth in SEQ ID NO: 43. In certain embodiments, the drug is trimethoprim, analogs thereof, or derivatives thereof. In certain embodiments, the drug-destabilized signaling domain has a FK506 binding protein destabilization domain (FKBP-DD). In certain embodiments, the drug is Shield-1 (Shld1), analogs thereof, or derivatives thereof.

In certain embodiments, the presently disclosed subject matter is directed to engineered immune cells comprising:
(a) a first nucleic acid construct comprising a nucleic acid sequence encoding a CAR; and
(b) a second nucleic acid construct comprising a nucleic acid sequence encoding: (1) a nuclear factor of activated T cells (NFAT) inhibitor peptide or NFkB inhibitor peptide; and a (2) a carrier protein; and
   wherein the second nucleic acid construct comprises a promoter element that regulates expression of the inhibitor peptide. In certain embodiments, the promoter element is a NFAT responsive promoter element or an NFkB responsive promoter element. In certain embodiments, the NFAT responsive promoter element comprises the sequence set forth in SEQ ID No: 48. In certain embodiments, the NFkB responsive promoter element comprises the sequence set forth in SEQ ID No: 49.

In certain embodiments, the presently disclosed subject matter is directed to engineered immune cells comprising:
(a) a first nucleic acid construct comprising a nucleic acid sequence encoding a CAR; and
(b) a second nucleic acid construct comprising a nucleic acid sequence encoding: (1) a nuclear factor of activated T cells (NFAT) inhibitor peptide or NFkB inhibitor peptide; and a (2) a carrier protein; and
   wherein the inhibitory peptide comprises the amino acid sequence set forth in any one of SEQ ID NOs: 70-93.

In certain embodiments, the presently disclosed subject matter is directed to methods for treating a disease comprising providing to a subject in need thereof:
(a) a population of modified cells comprising a system as described herein;
(b) a cell modified according to a method described herein; or
(c) an engineered immune cell as described herein.
   In certain embodiments, the subject is a human subject. In certain embodiments, the disease is a cancer, an autoimmune disease, an inflammatory disease, or a graft versus-host disease. In certain embodiments, the cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma. In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain embodiments, the lymphoma is Hodgkin's lymphoma or non-Hodgkin's lymphoma. In certain embodiments, the non-Hodgkin's lymphoma is B-cell non-Hodgkin's lymphoma or T-cell non-Hodgkin's lymphoma.

In certain embodiments, the cancer comprises cells expressing CD19 or CD20. In certain embodiments, the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LlCAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11

### BRIEF DESCRIPTION OF THE DRAWINGS

FIGS. 1A-1B depict exemplary approaches for improving immune cell, e.g., T cell, health and function. FIG. 1A illustrates an exemplary approach for inhibiting transcription factor NFAT activation by calcineurin in CAR expressing cells. This approach uses a VIVIT peptide appended to a “carrier” protein (e.g., enhanced green fluorescence protein) and expressed as a VIVIT-carrier fusion protein. Binding of the VIVIT peptide motif in the fusion protein to calcineurin inhibits its ability to dephosphorylate NFAT, whereby nuclear translocation of NFAT is inhibited. As a result, induction of proteins involved in cell exhaustion and/or cell death (e.g., PD-1/PD-L1, Lag-3, CD200R, Tim-3, Fas) is inhibited. FIG. 1B provides exemplary peptides that may be used to inhibit NFAT signaling. In one example, VIVIT, variants of VIVIT (Vmut), or other conserved PxIxIT calcineurin binding sequences of NFAT can be used to sequester calcineurin and block its interaction with NFAT, thereby effectively inhibiting NFAT signaling. These inhibitory peptides can be expressed as a fusion protein, appended to the C-terminal or N-terminal ends of a suitable carrier protein such as for example, enhanced green fluorescence protein (EGFP) and glutathione-S-transferase (GSTA1), among others. In another example, a peptide derived from the autoinhibitory domain of calcineurin (ITSFEEAKGLDRINERMPPRRDAMP), denoted here as “DAMP” can also be used to inhibit calcineurin dependent NFAT signaling. Similar to the PxIxIT group of peptides, the DAMP group of peptides can also be expressed as a fusion protein, appended to the C-terminal or N-terminal ends of carrier proteins like EGFP, GSTA1, or others.

FIGS. 2A-2B depict another exemplary approach for improving T cell health and function, which uses drug-regulated inhibition of NFAT in CAR expressing cells. FIG. 2A illustrates using destabilized mutants of dihydrofolate reductase (DHFR-DD) fused to the NFAT inhibitory peptides described above in FIG. 1A to generate inhibitory peptides. Stable intracellular expression of these peptides requires the presence of a stabilizing drug, for example, trimethoprim (TMP), thereby allowing for drug regulated inhibition of NFAT and thus, drug regulated inhibition of proteins involved in cell exhaustion and/or cell death. Destabilized mutants of FK506 binding protein destabilization domain (FKBP-DD) can similarly be used to generate inhibitory peptide-FKBP-DD fusion proteins, stable expression of which require a stabilizing drug, for example, Shield-1 (Shld1). FIG. 2B illustrates degradation of a DHFR-DD/NFAT inhibitory peptide (e.g., VIVIT-DHFR-DD) in the absence of TMP. In the presence of TMP, VIVIT-DHFR-DD is stably expressed thereby enabling inhibition of calcineurin mediated NFAT signaling.

FIGS. 3A-3C depict exemplary approaches to achieve NFAT inhibition via a negative feedback loop. FIG. 3A illustrates an approach to create dual-vector-transduced cells by enabling single-step magnetic-activated cell sorting (MACS), where the approach utilizes a heterodimerizing leucine zipper pair encoded by two vectors: (1) a secreted affinity-tagged zipper and (2), a membrane-bound capture-zipper, and where one of the vectors can comprise an inhibitory peptide-carrier fusion. FIG. 3B is a vector map illustrating use of an NFAT responsive promoter (NFAT-response element) to drive NFAT-dependent expression of the inhibitory peptide-carrier fusion proteins (e.g., VIVIT-EGFP). This negative feedback loop inhibits excessive NFAT signaling in cells (e.g., T cells) encountering targets with high antigen expression for extended durations, while avoiding NFAT inhibition against weaker targets that induce NFAT to a lesser extent. FIG. 3C is a vector map illustrating use of an NFkB responsive promoter to drive NFkB-dependent expression of the inhibitory peptide-carrier fusion protein.

FIGS. 4A-4C depict yet another exemplary approach for improving T cell health and function by inhibiting NFAT signaling in CAR expressing cells. FIG. 4A illustrates tethering Calcineurin/NFAT inhibitory peptides to the cytoplasmic-facing domain of transmembrane proteins, including a heterodimerizing leucine zipper pair (e.g., a pair comprising a membrane bound capture zipper and a secreted zipper that is secreted outside the cell). FIG. 4B illustrates an exemplary capture zipper with a transmembrane domain and a cytoplasmic stalk to which is attached a calcineurin/NFAT inhibitory peptide (e.g., Vmut3). FIG. 4C illustrates another exemplary capture zipper with a transmembrane domain and a cytoplasmic stalk to which is attached a protein having three calcineurin/NFAT inhibitory peptides (e.g., Vmut3 3×).

FIGS. 5A-5D depict yet another exemplary approach for regulating T cell function by attenuating NFkB signaling. FIG. 5A illustrates the effects of degradation-resistant mutants of an NFkB inhibitor (e.g., NFKBIAm) on T cell survival, proliferation, and cytokine production. FIG. 5B illustrates NFKBIAm (or other degradation-resistant mutants or NFkB) fused to destabilized mutants of dihydrofolate reductase (DHFR-DD) to enable drug-regulated NFkB attenuation. Stable intracellular expression of NFKBIAm in these cells require the presence of a stabilizing drug (e.g., TMP), thereby allowing for drug regulated NFkB attenuation. Destabilized mutants of FK binding protein (FKBP-DD) can similarly be used to generate NFKBIAm-FKBP-DD fusion proteins, stable expression of which require a stabilizing drug (e.g., Shield-1). FIG. 5C illustrates degradation of a DHFR-DD-NFKBIAm in the absence of TMP. In the presence of TMP, DHFR-DD-NFKBIAm is stably expressed thereby achieving NFkB attenuation. FIG. 5D illustrates use of an NFAT responsive promoter to drive NFkB-dependent expression of NFkB-inhibiting proteins.

FIGS. 6A-6C show exemplary data for TMP inducible VIVIT peptide expression in T cells transfected with hemagglutinin tagged (HA-tag) VIVIT-DHFR-DD (see exemplary FIG. 2A). FIG. 6A shows FACS analysis of VIVIT-DHFR-DD expression in T cells in the absence (DMSO) or presence of TMP by HA-tag staining. FIG. 6B shows FACS analysis for expression of the T cell exhaustion regulator, PD-1, and NFAT-EGFP in the absence or presence of TMP. FIG. 6C shows quantitative assessment of NFAT-EGFP expression in the absence or presence of TMP.

FIGS. 7A-7G are show exemplary data for the effects of constitutive NFAT inhibition in Zip-sorted CD19-28z/CD20-28z dual-CAR T cells (see exemplary FIG. 4A) transduced with VIVIT-GFP, EGFP-DAMP, or EGFP constructs. FIG. 7A shows LAG-3 upregulation in Zip-sorted CD19-28z/CD20-28z dual-CAR T cells cultured with or without targets for 24 h. FIG. 7B shows PD-1 upregulation in the Zip-sorted CD19/28z/CD20/28z dual-CAR T cells cultured with or without targets for 24 h. FIG. 7C shows PD-1 and LAG-3 upregulation in the Zip-sorted CD19/28z/CD20/28z dual-CAR T cells stimulated for 24 h with anti-CD3/CD28. FIG. 7D shows a luciferase-based target cell lysis assay using the Zip-sorted CD19-28z/CD20-28z dual CAR T cells. FIG. 7E shows the results of bioluminescent imaging (BLI) of leukemia cells injected into BALB/c mice that received the Zip-sorted CD19/28z/CD20/28z dual-CAR T cells transduced with the indicated constructs. FIG. 7F shows the results of survival studies in the animals treated as described for FIG. 7E. FIG. 7G shows FACS analysis of CD45.1⁺ CAR T cells in peripheral blood on day 14.

FIGS. 8A-8E are exemplary data showing the effect of higher affinity PxIxIT calcineurin binding peptide sequences on NFAT inhibition. FIG. 8A shows PD-1 and LAG-3 down regulation after 24 h stimulation with anti-CD3/CD28 in T cells transduced with the indicated VIVIT mutants expressed as EGFP fusion peptides. FIG. 8B shows HA-tag staining in T cells transduced with vectors encoding HA-tagged human GSTA1, VIVIT-GSTA1, and Vmut3-GSTA1. FIG. 8C shows PD-1 upregulation as function of hCD8 reporter expression in T cells transduced with the indicated vectors and stimulated for 24 h with CD3/CD28. FIG. 8D shows PD-1 levels as function of anti-CD3/CD28 in T cells transduced with the indicated vectors and stimulated for 24 h. FIG. 8E shows PD-1 upregulation in T cells transduced with the indicated vectors and stimulated for 24 h in the presence or absence of BM185-CD19 targets with varying concentrations of CD3/CD28 for 24 h as depicted. Unstimulated cells were assigned an arbitrary value of 10⁻² μg/mL stimulation. As illustrated in FIG. 8E, PD-1 and LAG-3 upregulation on Zip-sorted CD1928z/CD2028z BALB/c dual-CAR T cells co-transduced with VIVIT-GSTA1, Vmut3-GSTA1, or GSTA1 control vectors following 24 h stimulation with BM185-CD19 targets or no stimulation. Vmut3-GSTA1 enables potent inhibition of inhibitory receptor upregulation in contrast to VIVIT-GSTA1, which has insufficient affinity and expression to inhibit in this context.

FIG. 9 shows FACS analysis of EGFP and PD-1 expression in NFAT-VIVIT-EGFP or control NFAT-EGFP CAR T cells (see illustrations in FIGS. 1A and 3A) incubated for 2 d with or without C1498-CD19 targets added every 24 h (E:T 1:1, two additions).

FIGS. 10A-10B show exemplary data depicting drug-dependent inhibition of NFAT and AP-1 signaling. FIG. 10A shows PD-1 and LAG-3 expression in Zip-sorted CD19-28z/CD20-28z dual-CAR T cells transduced with Vmut3-DHFR-DD-E2A-hCD8 vector, cultured 2 d with TMP and stimulated for 24 h with BM185-CD19 targets. FIG. 10B shows induction of NFAT and AP-1 reporters in the T cells described in FIG. 10A.

FIG. 11 shows exemplary use of NFkB-inducible NFAT-inhibitor expression (see FIG. 3C) for inhibiting PD-1 and Lag-3 upregulation in transduced T cells stimulated for 24 h with CD3/CD28.

FIGS. 12A-12D shows exemplary use of capture-zipper of a leucine zipper sorting system for presenting calcineurin/NFAT inhibitory peptide (see FIG. 4A). FIG. 12A shows PD-1 expression following CD3/CD28 stimulation in CD19-28z/CD20-28z dual-CAR T cells transduced with a capture zipper conjugated at its C-terminus with a NFAT inhibitory peptide (e.g., Vmut3 peptide). FIG. 12B shows Lag-3 expression following CD3/CD28 stimulation in the transduced dual-CAR T cells. FIG. 12C shows PD-1 expression following co-culturing the transduced dual-CAR T cells with BM185-CD19 targets. FIG. 12D shows Lag-3 expression following co-culturing the transduced dual-CAR T cells with BM185-CD19 targets.

FIGS. 13A-13F shows exemplary use of triplet repeats of calcineurin/NFAT inhibitory peptides for enhancing inhibition of CAR and TCR-stimulated upregulation of PD-1 and Lag-3. FIG. 13A shows FACS analysis for inhibition of PD-1 and Lag-3 induction in T cells expressing Vmut3 (1×) and triplet repeat Vmut3 (3×) calcineurin/NFAT inhibitory peptides using the capture-zipper format (see FIG. 4A) following overnight stimulation with CD3/CD28. A percent of population chart derived from the FACS data is also shown. FIG. 13B shows mean fluorescence intensity (MFI) of PD-1 and Lag-3 on the cells described in FIG. 13A. FIG. 13C shows relative inhibition of anti-CD3/CD28 stimulated PD-1 and Lag-3 induction by Vmut3 and Vmut3 3× capture-zippers. FIG. 13D shows FACS analysis for inhibition of PD-1 and Lag-3 induction in T cells expressing Vmut3 (1×) and Vmut3 (3×) calcineurin/NFAT inhibitory peptides following overnight stimulation with BM185-CD19 targets. FIG. 13E shows MFI of PD-1 and Lag-3 in the cells described in FIG. 13D. FIG. 13F shows relative inhibition of antigen stimulated PD-1 and Lag-3 induction by Vmut3 (1×) and Vmut3 (3×) capture-zippers. A percent of population chart derived from the FACS data is also shown.

FIGS. 14A-14E shows exemplary use of vectors-encoding NFkB inhibitory protein NFKBIAm to abrogates antigen-independent proliferation associated with tonic signaling in dual-CAR T cells. FIG. 14A shows vector maps for CD19-4-1BB CAR (CD19BBz, secreted-zipper) and CD20-4-1BB CAR (CD20BBz, capture-zipper) vectors encoding the NFκB inhibitor, NFKBIAm. FIG. 14B shows FACS analysis of HA-tagged NFKBIAm expression in the dual transduced CAR T cells described in FIG. 14A. FIG. 14C shows FACS analysis of NFκB induction (EGFP) in zip-sorted CD19/CD20 dual-CAR T cells co-transduced with an SV40-BFP-NFκB-dEGFP reporter vector and stimulated for 24 h with BM185-CD20 targets (E:T=1:1). FIG. 14D shows NFκB reporter activity in T cells transduced with vectors encoding CD20BBz±NFKBIAm, followed by co-culture with BM185-CD20 targets for 24 h (E:T=1:1). FIG. 14E shows target-stimulated spontaneous carboxyfluorescein diacetate, succinimidyl ester (CFSE) diluted and absolute T cell counts of dual-CAR T cells following 3 d culture with irradiated targets at (E:T=1:3).

FIGS. 15A-15E show reversal of immune phenotype induced by tonic signaling in dual-4-1BB CAR T cells following NFκB inhibition. FIG. 15A shows expression of TIM-3, CD44/CD62L and CD39 in CD4+ and CD8+ dual-4-1BB CAR T cells. FIG. 15B shows FACS analysis of immunophenotype in unstimulated day 5 cultured Zip-sorted CD19/CD20 dual-CAR T cells (First generation (FG), NFKBIAm BB/BB, and BB/BB). FIG. 15C shows expression of CD200 in the dual CAR T cells described for FIG. 5B. FIG. 15D shows quantitation of total cellular ROS production in the T cells described in FIGS. 15A-15C using CM-H₂DCFDA. FIG. 15E shows the results of FACS analysis for transcription factors and apoptosis-associated proteins in T cells described in FIGS. 15A-15C.

FIGS. 16A-16D show prevention of toxicity and cytokine release following NFKB inhibition in dual-4-1BB-CAR T cells. FIG. 16A shows bioluminescence from BM185 leukemic cells injected into BALB/c mice, which were administered sorted CD19BBz/CD20BBz dual CAR BALB/c T cells or, CD19BBz/CD20BBz CAR T cells transduced with the NFKBIAm vector. FIG. 16B shows the results of survival studies performed in the animals treated as described in FIG. 16A. FIG. 16C shows serial assessment of weights post BM185 leukemia cell injection for the animals treated as described in FIG. 16A. FIG. 16D shows the results of serum cytokine analysis on day 6 post BM185 leukemia cell injection.

FIGS. 17A-17C show prevention of toxicity and cytokine release following NFKB inhibition in CD24-CAR T cells. FIG. 17A shows tumor fluorescence, body weights and survival of BALB/c mice injected with BM185 leukemic cells followed by administration with CD24-CAR T cells. FIG. 17B shows survival of BALB/c mice injected with BM185 leukemic cells followed by administration with CD24-CAR T cells or CD24-CAR T cells transduced with the NFKBIAm vector. FIG. 17C shows the results of serum cytokine analysis on day 6 post BM185 leukemia cell injection in mice administered the indicated CAR T cells.

FIG. 18 shows exemplary use of NFAT-inducible NFKBIAm expression (see FIG. 5D) for inhibiting NFκB-upregulation of CD200.

FIG. 19 shows exemplary use of drug inducible NFKBIAm expression for inhibiting of tonic T cell proliferation associated in dual-4-1BB CAR T cells. Zip-sorted CD19BBz/CD20BBz dual-CAR T cells, CD19BBz/CD20BBz dual-CAR T cells transduced with the NFKBIAm vector, or the DHFR-DD-NFKBIAm vector were incubated with TMP for 1 or 3 days, after which T cell counts and NFKBIAm expression (HA-tag) were evaluated.

### DETAILED DESCRIPTION

The present disclosure is directed to systems and methods to improve the function of CAR expressing immune cells, e.g., CAR T cells, by inhibiting NFAT and/or NFkB signaling. For example, the systems of the present disclosure enable the generation of T cells engineered to express a single CAR or multiple combinations of CARs (multi-CAR) and one or more inhibitors of NFAT and/or NFkB signaling. In certain embodiments, the presently disclosed subject matter relates to the use of a leucine zipper-based system in connection with inhibition of NFAT and/or NFkB signaling, where the leucine zipper-based system enables single-step immunomagnetic sorting of cells transduced with two vectors with the goal of doubling the amount of genetic information delivered and promoting enhanced transgene expression.

The platform described herein facilitates generation of immune cells, e.g., T cells, expressing combinations of many transgenes at high expression levels able to simultaneously overcome multiple challenges faced by CAR expressing immune cells, e.g., CAR T cells. This platform exhibits remarkable versatility, allowing generation of capture-zippers from a wide array of molecules. As outlined herein, this system can be used to overcome antigen-loss escape and tumor-associated immune suppression strategies, inhibit CRS, and attenuate tonic CAR signaling-induced dysfunction.

The subject matter of the present disclosure is described with reference to the Figures. It should be understood that numerous specific details, relationships, and methods are set forth in this Detailed Description, Examples, and accompanying Figures to provide a more complete understanding of the subject matter disclosed herein.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other instances “comprising,” “consisting of”, and “consisting essentially of,” the instances or elements presented herein, whether explicitly set forth or not.

For the recitation of numeric ranges herein, each intervening number within the range is explicitly contemplated with the same degree of precision. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein the term “inhibitory peptide” or “inhibitory polypeptide” refers to any peptide or peptide expressed as fusion product with a carrier polypeptide or protein.

As used herein, a “linker” refers to a functional group (e.g., chemical or polypeptide) that covalently attaches two or more polypeptides or nucleic acids so that they are connected to one another. In certain embodiments, the linker comprises one or more amino acids used to couple two polypeptides together (e.g., to couple V_{H} and V_{L} domains or to couple two dimerization domains). The linker can be usually rich in glycine for flexibility, as well as serine or threonine for solubility.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence operably linked to appropriate nucleic acid sequences necessary for the expression of the coding sequence in a particular host organism. Nucleic acid sequences necessary for expression in prokaryotes usually include a promoter, an operator (optional), and a ribosome binding site, often along with other sequences. Nucleic acid sequences necessary for expression in eukaryotic cells can include, but are not limited to, promoters, enhancers, and termination and polyadenylation signals.

In certain embodiments, nucleic acid molecules useful in the presently disclosed subject matter include nucleic acid molecules that encode an antibody or an antigen-binding fragment thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule.

As used herein, the term “disease” refers to any condition or disorder that damages or interferes with the normal function of a cell, tissue, or organ. Examples of diseases include neoplasia or pathogenic infection of a cell, tissue, or organ.

An “effective amount” (or “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The dose comprising an effective amount is generally determined by the physician on a case-by-case basis and making such a determination is within the level of ordinary skill in the art. Several factors are typically taken into account when determining an appropriate dosage to achieve an effective amount. These factors include age, sex and weight of the subject, the condition being treated, the severity of the condition and the form and effective concentration of the cells (e.g., engineered immune cells) administered.

As used herein, the term “neoplasm” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of skin, bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, spinal cord, spleen, stomach, testes, thymus, thyroid, trachea, urogenital tract, ureter, urethra, uterus, and vagina, or a tissue or cell type thereof. Neoplasia include cancers, such as melanoma, sarcomas, carcinomas, or plasmacytomas (malignant tumor of the plasma cells).

As used herein, the term “immunoresponsive cell” refers to a cell that functions in an immune response, and includes a progenitor of such cell, and a progeny of such cell.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” polypeptide is sufficiently free of other materials such that any impurities do not materially affect the biological properties of the polypeptide or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or polypeptide gives rise to essentially one band in an electrophoretic gel. For a polypeptide that can be subjected to modifications, for example, phosphorylation or glycosylation, different modifications may give rise to different isolated polypeptides, which can be separately purified.

As used herein, the term “secreted” refers to a polypeptide that is released from a cell via the secretory pathway through the endoplasmic reticulum, Golgi apparatus, and as a vesicle that transiently fuses at the cell plasma membrane, releasing the polypeptides outside of the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, bispecific antibodies; chimeric antibodies; Fab, Fab′, single chain V region fragments (scFv), fusion polypeptides, and unconventional antibodies. In certain embodiments, an antibody is a glycopolypeptide comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, the term “single-chain variable fragment” or “scFv” is a fusion polypeptide of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., about 10, 15, 20, 25 amino acids), which connects the N-terminus of the V_{H} with the Cterminus of the V_{L}, or the C-terminus of the V_{H} with the N-terminus of the V_{L}.

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

In certain non-limiting embodiments, an intracellular signaling domain of a CAR or a ZipR CAR comprises a CD3ζ polypeptide, which can activate or stimulate a cell (e.g., a cell of the lymphoid lineage, e.g., a T cell). CD3ζ comprises 3 immunoreceptor tyrosine-based activation motifs (ITAMs) and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain non-limiting embodiments, a CAR or a ZipR CAR can also comprise a spacer/hinge region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and fragments of CD3, a fragment of a CD28 polypeptide, a fragment of a CD8 polypeptide, a variant thereof, or a synthetic spacer sequence.

As used herein, “costimulatory molecules” refer to cell surface molecules other than antigen receptors or their ligands that are required for a response of lymphocytes to antigen. The at least one co-stimulatory signaling region can include a CD28 polypeptide (e.g., intracellular domain of CD28 or a fragment thereof), a 4-1BB polypeptide (e.g., intracellular domain of 4-1BB or a fragment thereof), an OX40 polypeptide (e.g., intracellular domain of OX40 or a fragment thereof), an ICOS polypeptide (e.g., intracellular domain of ICOS or a fragment thereof), a DAP-10 polypeptide (e.g., intracellular domain of DAP10 or a fragment thereof), or a combination thereof. The co-stimulatory molecule can bind to a co-stimulatory ligand. As used herein, the term a “co-stimulatory ligand” refers to a polypeptide expressed on cell surface that upon binding to its receptor produces a co-stimulatory response, i.e., an intracellular response that effects the stimulation provided by an activating signaling domain (e.g., a CD3ζ signaling domain). Non-limiting examples of co-stimulatory ligands include tumor necrosis factor (TNF) family members, immunoglobulin (Ig) superfamily members, or combination thereof. the co-stimulatory ligand is selected from the group consisting of tumor necrosis factor (TNF) family members, immunoglobulin (Ig) superfamily members, and combinations thereof. Non-limiting examples of TNF family member include 4-1BBL, OX40L, CD70, GITRL, CD40L, RANK, GITR, LTBR, HVEM, BAFF-R, TACI, BCMA, TROY, and CD30L. Non-limiting examples of Ig superfamily member include CD80, CD86, and ICOSLG. For example, 4-1BBL may bind to 4-1BB for providing an intracellular signal that in combination with a CAR signal induces an effector cell function of the CAR⁺ T cell. CARs comprising an intracellular signaling domain that comprises a co-stimulatory signaling region comprising a 4-1BB, ICOS or DAP-10 co-stimulatory signaling domain are disclosed in U.S. Pat. No. 7,446,190, which is herein incorporated by reference in its entirety.

As used herein, the term “multimerization” refers to the formation of multimers (including dimers). Multimerization includes dimerization.

As used herein, the term “a conservative sequence modification” refers to an amino acid modification that does not significantly affect or alter the binding characteristics of the presently disclosed polypeptide (e.g., the extracellular antigen-binding domain of the polypeptide) comprising the amino acid sequence. Conservative modifications can include amino acid substitutions, additions and deletions. Modifications can be introduced into the human scFv of the presently disclosed polypeptide by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function using the functional assays described herein. In certain embodiments, no more than one, no more than two, no more than three, no more than four, no more than five residues within a specified sequence or a CDR region are altered.

As used herein, “very low” expression of a ligand or receptor corresponds to less than 2-fold increase in mean fluorescence intensity (MFI) shift when compared with a negative control cell line. As used herein, “very high” expression of a ligand or receptor corresponds to greater than 20-fold increase in mean fluorescence intensity (MFI) shift when compared with a negative control cell line.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal polypeptide processing (e.g., removal of amino acids from the nascent polypeptide that are not required for biological activity or removal of amino acids by alternative mRNA splicing or alternative polypeptide processing events).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST polypeptide searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

### 2. Exemplary Sequences

Table 1 lists exemplary sequences for the elements and constructs of the system in the presently disclosed subject matter.

TABLE 1
Exemplary amino acid sequences for elements and constructs of the system in
the presently disclosed subject matter

| | | SEQ |
|---|---|---|
| Description | Sequence | ID NO: |
| Linker1 | GSGRSG | 1 |
| Linker2 | GGGGSGGGS | 2 |
| Linker3 | GGGGSGGGGSGGGGSTS | 3 |
| Linker4 | GPPVASGSGRSG | 4 |
| Linker5 | GGGGSGGGSGSL | 5 |
| Linker6 | GGGGSGGGSELPTQGTFSNVSTNVSGGGGSGGGSRSG | 6 |
| Linker7 | TSGGGGSGGGSG | 7 |
| Linker8 | TSGGGGSGGGSGTG | 8 |
| Linker9 | GGGGSGGGSGTG | 9 |
| Linker10 | STGGGGSGGGS | 10 |
| Linker11 | GSGGGGSGGGS | 11 |
| Linker12 | GTSRAKRGSG | 12 |
| Linker13 | GGGGSGTG | 13 |
| Linker14 | GTSRAKRGS | 14 |
| Linker15 | ALGGSGGGS | 15 |
| Linker16 | GGGGSTS | 16 |
| Linker17 | SGGGGSD | 17 |
| Linker18 | GSSGSG | 18 |
| Linker19 | GSGGTR | 19 |
| Linker20 | GSGTR | 20 |
| Linker21 | TSGSG | 21 |
| Linker22 | GSGGS | 22 |
| interchain linker | GSTGGGGSGGGGSGGGGS | 23 |
| VIVIT peptide | MAGPHPVIVITGPHEEGPPVAS | 24 |
| Vmut1 | MAGPVPRIIITKPYEEGPPVAS | 25 |
| Vmut2 | MAGPVPRIIITKPDEEGPPVAS | 26 |
| Vmut3 | MAGPVPRIIITKPEEEGPPVAS | 27 |
| Vmut3 3x | MAGPVPRIIITKPEEEGSTGGGGSGGGGSGGGGSMAGP | 28 |
| | VPRIIITKPEEEGSTGGGGSGGGGSGGGGSMAGPVPRIII | |
| | TKPEEE | |
| Vmut5 | MASGLLPRIIITPPYELGPPVAS | 29 |
| DAMP | ITSFEEAKGLDRINERMPPRRDAMP | 30 |
| linker + DAMP peptide | GGGGSGGGSGSLITSFEEAKGLDRINERMPPRRDAMP | 31 |
| (linker underlined, | | |
| DAMP peptide in bold) | | |
| NFKBIAm (mutant IkBa) | MFQPAGHGQDWAMEGPRDGLKKERLVDDRHDAGLD | 32 |
| | AMKDEEYEQMVKELREIRLQPQEAPLAAEPWKQQLTE | |
| | DGDSFLHLAIIHEEKPLTMEVIGQVKGDLAFLNFQNNLQ | |
| | QTPLHLAVITNQPGIAEALLKAGCDPELRDFRGNTPLHL | |
| | ACEQGCLASVAVLTQTCTPQHLHSVLQATNYNGHTCL | |
| | HLASIHGYLAIVEHLVTLGADVNAQEPCNGRTALHLAV | |
| | DLQNPDLVSLLLKCGADVNRVTYQGYSPYQLTWGRPS | |
| | TRIQQQLGQLTLENLQMLPEAEDEEAYDAEAEFAEDEL | |
| | PDDDCVFGGQRLTL | |
| NFKBIAm DHFR-DD | YPYDVPDYAGYPYDVPDYAGYPYDVPDYAGSGMFQP | 33 |
| | AGHGQDWAMEGPRDGLKKERLVDDRHDAGLDAMKD | |
| | EEYEQMVKELREIRLQPQEAPLAAEPWKQQLTEDGDSF | |
| | LHLAIIHEEKPLTMEVIGQVKGDLAFLNFQNNLQQTPLH | |
| | LAVITNQPGIAEALLKAGCDPELRDFRGNTPLHLACEQ | |
| | GCLASVAVLTQTCTPQHLHSVLQATNYNGHTCLHLASI | |
| | HGYLAIVEHLVTLGADVNAQEPCNGRTALHLAVDLQN | |
| | PDLVSLLLKCGADVNRVTYQGYSPYQLTWGRPSTRIQQ | |
| | QLGQLTLENLQMLPEAEDEEAYDAEAEFAEDELPDDD | |
| | CVFGGQRLTLGSGTRGSTGGGGSGGGGSGGGGSISLIA | |
| | ALAVDHVIGMETVMPWNLPADLAWFKRNTLNKPVIM | |
| | GRHTWESIGRPLPGRKNIILSSQPSTDDRVTWVKSVDEA | |
| | IAACGDVPEIMVIGGGRVYEQFLPKAQKLYLTHIDAEV | |
| | EGDTHFPDYEPDDWESVFSEFHDADAQNSHSYCFEILE | |
| | RR | |
| EE12RR345L zipper | LEIEAAFLERENTALETRVAELRQRVQRLRNRVSQYRT | 34 |
| | RYGPLGGGK | |
| RR12EE345L zipper | LEIRAAFLRQRNTALRTEVAELEQEVQRLENEVSQYET | 35 |
| | RYGPLGGGK | |
| 3N RR EE blocked zipper | LEIEAAFLRQENTALRTRVAELEQEVQRLENEVSQYET | 36 |
| | RYGPLGGGKGGGGSGGGGSGGGGSGGGSLEIEAAFLE | |
| | RENTALETRVAELRQRVQRLRNRVSQYRTRYGPLGGG | |
| | K | |
| RR EE blocked zipper | LEIRAAFLRQRNTALRTEVAELEQEVQRLENEVSQYET | 37 |
| | RYGPLGGGKGGGGSGGGGSGGGGSGGGSLEIEAAFLE | |
| | RENTALETRVAELRQRVQRLRNRVSQYRTRYGPLGGG | |
| | K | |
| 3N mutant zipper (bolded | LEIEAAFLRQENTALRTRVAELEQEVQRLENEVSQYET | 38 |
| are R/E substitutions) | RYGPLGGGK | |
| 3x HA tag | YPYDVPDYAGYPYDVPDYAGYPYDVPDYA | 39 |
| twin Strep Tag | SAWSHPQFEKGGGSGGGSGGSAWSHPQFEKGGGGSGG | 40 |
| | GS | |
| tagBFP | MSELIKENMHMKLYMEGTVDNHHFKCTSEGEGKPYEG | 41 |
| | TQTMRIKVVEGGPLPFAFDILATSFLYGSKTFINHTQGIP | |
| | DFFKQSFPEGFTWERVTTYEDGGVLTATQDTSLQDGCL | |
| | IYNVKIRGVNFTSNGPVMQKKTLGWEAFTETLYPADG | |
| | GLEGRNDMALKLVGGSHLIANIKTTYRSKKPAKNLKM | |
| | PGVYYVDYRLERIKEANNETYVEQHEVAVARYCDLPS | |
| | KLGHKLN | |
| EGFP | MVSKGEELFTGVVPILVELDGDVNGHKFSVSGEGEGDA | 42 |
| | TYGKLTLKFICTTGKLPVPWPTLVTTLTYGVQCFSRYPD | |
| | HMKQHDFFKSAMPEGYVQERTIFFKDDGNYKTRAEVK | |
| | FEGDTLVNRIELKGIDFKEDGNILGHKLEYNYNSHNVYI | |
| | MADKQKNGIKVNFKIRHNIEDGSVQLADHYQQNTPIGD | |
| | GPVLLPDNHYLSTQSALSKDPNEKRDHMVLLEFVTAA | |
| | GITLGMDELYK | |
| DHFR-DD | MISLIAALAVDYVIGMENAMPWNLPADLAWFKRNTLN | 43 |
| | KPVIMGRHTWESIGRPLPGRKNIILSSQPSTDDRVTWVK | |
| | SVDEAIAACGDVPEIMVIGGGR VIEQFLPKAQKLYLTHI | |
| | DAEVEGDTHFPDYEPDDWESVFSEFHDADAQNSHSYC | |
| | FEILERR | |
| E2A peptide | QCTNYALLKLAGDVESNPGP | 44 |
| human CD8 alpha | MALPVTALLLPLALLLHAARPSQFRVSPLDRTWNLGET | 45 |
| | VELKCQVLLSNPTSGCSWLFQPRGAAASPTFLLYLSQN | |
| | KPKAAEGLDTQRFSGKRLGDTFVLTLSDFRRENEGYYF | |
| | CSALSNSIMYFSHFVPVFLPAKPTTTPAPRPPTPAPTIAS | |
| | QPLSLRPEACRPAAGGAVHTRGLDFACDIYIWAPLAGT | |
| | CGVLLLSLVITLYCNHRNRRRVCKCPRPVVKSGDKPSL | |
| | SARYV | |
| glutathione-S- | MAEKPKLHYFNARGRMESTRWLLAAAGVEFEEKFIKS | 46 |
| transferase (GSTA1) | AEDLDKLRNDGYLMFQQVPMVEIDGMKLVQTRAILNY | |
| | IASKYNLYGKDIKERALIDMYIEGIADLGEMILLLPVCPP | |
| | EEKDAKLALIKEKIKNRYFPAFEKVLKSHGQDYLVGNK | |
| | LSRADIHLVELLYYVEELDSSLISSFPLLKALKTRISNLPT | |
| | VKKFLQPGSPRKPPMDEKSLEEARKIFRF | |
| SFFV promoter | GTAACGCCATTTTGCAAGGCATGGAAAAATACCAAA | 47 |
| | CCAAGAATAGAGAAGTTCAGATCAAGGGCGGGTACA | |
| | TGAAAATAGCTAACGTTGGGCCAAACAGGATATCTG | |
| | CGGTGAGCAGTTTCGGCCCCGGCCCGGGGCCAAGAA | |
| | CAGATGGTCACCGCAGTTTCGGCCCCGGCCCGAGGC | |
| | CAAGAACAGATGGTCCCCAGATATGGCCCAACCCTC | |
| | AGCAGTTTCTTAAGACCCATCAGATGTTTCCAGGCTC | |
| | CCCCAAGGACCTGAAATGACCCTGCGCCTTATTTGAA | |
| | TTAACCAATCAGCCTGCTTCTCGCTTCTGTTCGCGCG | |
| | CTTCTGCTTCCCGAGCTCTATAAAAGAGCTCACAACC | |
| | CCTCACTCGGCGCGCCAGTCCTCCGACAGACTGAGTC | |
| | G | |
| NFAT response element | GGAGGAAAAACTGTTTCATACAGAAGGCGTCAATTA | 48 |
| | GGAGGAAAAACTGTTTCATACAGAAGGCGTCAATTA | |
| | GGAGGAAAAACTGTTTCATACAGAAGGCGTCAATTG | |
| | GTCCCATCGAATTAGGAGGAAAAACTGTTTCATACA | |
| | GAAGGCGTCAATTAGGAGGAAAAACTGTTTCATACA | |
| | GAAGGCGTCAATTAGGAGGAAAAACTGTTTCATACA | |
| | GAAGGCGTCAATTGGTCCCGGGACATTTTGACACCCC | |
| | CATAATATTTTTCCAGAATTAACAGTATAAATTGCAT | |
| | CTCTTGTTCAAGAGTTCCCTATCACTCTCTTTAATCAC | |
| | TACTCACAGTAACCTCAACTCCTG | |
| NFKB response element | gggactttccgcttggggactttccgctggggactttccgctggggactttccgctggg | 49 |
| | gactttccgcggagactctagagggtatataatggaagctcgaattccag | |
| poly(A) signal | AATAAAATATCTTTATTTTCATTACATCTGTGTGTTGG | 50 |
| | TTTTTTGTGTG | |
| R2-dual hCD20 cyclic | CPYSNPSLCSGGGGSSGGGGSDCPYSNPSLCGGGGSGG | 51 |
| mimotope with | GS | |
| intervening linkers | | |
| PD-1 hinge | TERILETSTRYPSPSPKPEGRFQGM | 52 |
| CD28TM | FWALVVVAGVLFCYGLLVTVALCVIWT | 53 |
| CD28 hinge | IEFMYPPPYLDNERSNGTIIHIKEKHLCHTQSSPKL | 54 |
| CD28 cytoplasmic | NSRRNRLLQSDYMNMTPRRPGLTRKPYQPYAPARDFA | 55 |
| | AYRP | |
| human 4-1BB cytoplasmic | KRGRKKLLYIFKQPFMRPVQTTQEEDGCSCRFPEEEEG | 56 |
| domain | GCEL | |
| CD3zeta delta | RAKFSRSAETAANLQDTFDALHMQTLAPR | 57 |
| CD3zeta | RAKFSRSAETAANLQDPNQLYNELNLGRREEYDVLEK | 58 |
| | KRARDPEMGGKQQRRRNPQEGVYNALQKDKMAEAYS | |
| | EIGTKGERRRGKGHDGLYQGLSTATKDTYDALHMQTL | |
| | APR | |
| P2A | ATNFSLLKQAGDVEENPGP | 59 |
| Q2 CD34 zipper | ELPTQGTFSNVSTNVSGGGGSGGGSELPTQGTFSNVSTN | 60 |
| | VS | |
| iCaspase9 | MGVQVETISPGDGRTFPKRGQTCVVHYTGMLEDGKKV | 61 |
| | DSSRDRNKPFKFMLGKQEVIRGWEEGVAQMSVGQRAK | |
| | LTISPDYAYGATGHPGIIPPHATLVFDVELLKLESGGGS | |
| | GVDGFGDVGALESLRGNADLAYILSMEPCGHCLIINNV | |
| | NFCRESGLRTRTGSNIDCEKLRRRFSSLHFMVEVKGDLT | |
| | AKKMVLALLELARQDHGALDCCVVVILSHGCQASHLQ | |
| | FPGAVYGTDGCPVSVEKIVNIFNGTSCPSLGGKPKLFFIQ | |
| | ACGGEQKDHGFEVASTSPEDESPGSNPEPDATPFQEGLR | |
| | TFDQLDAISSLPTPSDIFVSYSTFPGFVSWRDPKSGSWY | |
| | VETLDDIFEQWAHSEDLQSLLLRVANAVSVKGIYKQMP | |
| | GCFNFLRKKLFFKTSVD | |
| CD8 hinge | STTTKPVLRTPSPVHPTGTSQPQRPEDCRPRGSVKGTGL | 62 |
| | DFACD | |
| CD8TM | IYIWAPLAGICVALLLSLIITLICY | 63 |
| F2A | VKQTLNFDLLKLAGDVESNPGP | 64 |
| FLAG RR zipper | DYKDDDDKLEIRAAFLRQRNTALRTEVAELEQEVQRLE | 65 |
| | NEVSQYETRYGPLGGGK | |
| Thy 1.1 | QKVTSLTACLVNQNLRLDCRHENNTKDNSIQHEFSLTR | 66 |
| | EKRKHVLSGTLGIPEHTYRSRVTLSNQPYIKVLTLANFT | |
| | TKDEGDYFCELRVSGANPMSSNKSISVYRDKLVKCGGI | |
| | SLLVQNTSWMLLLLLSLSLLQALDFISL | |
| clone M1/69 CD24 scFv | EVKLQESGPGLVKPSLTLSLTCTVSGFSLNNYAVIWVR | 67 |
| | QPPGKGLEWMGVIWSDGKTNYNSALKSRLSISRDTSKS | |
| | QVFLKMNNLQTEDTAMYFCARSSYYSSWFDSWGQGV | |
| | MVTVSSGGGGSGGGGSGGGGSDIQLTQSPSFLSASVGD | |
| | RVTLSCKASQNINKYLAWYQQKLGEAPKLLIYNANSLQ | |
| | TGIPSRFSGSGSGTDFTLTISSLQPEDVATYFCLQHNSW | |
| | WTLGGGTKLELKR | |
| 1D3 CD19 scFv-VH- | EVQLQQSGAELVRPGTSVKLSCKVSGDTITFYYMHFVK | 68 |
| (G4S)3-VL (CD19 scFv) | QRPGQGLEWIGRIDPEDESTKYSEKFKNKATLTADTSSN | |
| | TAYLKLSSLTSEDTATYFCIYGGYYFDYWGQGVMVTV | |
| | SSGGGGSGGGGSGGGGSDIQMTQSPASLSTSLGETVTIQ | |
| | CQASEDIYSGLAWYQQKPGKSPQLLIYGASDLQDGVPS | |
| | RFSGSGSGTQYSLKITSMQTEDEGVYFCQQGLTYPRTF | |
| | GGGTKLELKR | |
| 18B12 scFv mCD20 | TMASPLTRFLSLNLLLLGESIILGSGEAQIVMSQSPAILSA | 69 |
| targeting-Patent: | SPGEKVTMTCRARSSVSYIHWYQQKPGSSPKPWIYATS | |
| WO2007064911A1 | NLASGVPGRFSGSGSGTSYSLTITRVEAEDAATYYCQQ | |
| | WSSKPPTFGGGTKLEIKRGGGGSGGGGSGGGGSQVQL | |
| | QQPGAELVRPGTSVKLSCKASGYTFTSYWMHWIKQRP | |
| | GQGLEWIGVIDPSDNYTKYNQKFKGKATLTVDTSSSTA | |
| | YMQLSSLTSEDSAVYFCAREGYYGSSPWFAYWGQGTL | |
| | VTVSS | |
| VIVIT-EGFP | MAGPHPVIVITGPHEEGPPVASGSGRSGMVSKGEELFTG | 70 |
| | VVPILVELDGDVNGHKFSVSGEGEGDATYGKLTLKFIC | |
| | TTGKLPVPWPTLVTTLTYGVQCFSRYPDHMKQHDFFKS | |
| | AMPEGYVQERTIFFKDDGNYKTRAEVKFEGDTLVNRIE | |
| | LKGIDFKEDGNILGHKLEYNYNSHNVYIMADKQKNGIK | |
| | VNFKIRHNIEDGSVQLADHYQQNTPIGDGPVLLPDNHY | |
| | LSTQSALSKDPNEKRDHMVLLEFVTAAGITLGMDELYK | |
| VIVIT 3xHA DHFR-DD | MAGPHPVIVITGPHEEGPPVASGSGRSGYPYDVPDYAG | 71 |
| N type hCD8 | YPYDVPDYAGYPYDVPDYAGSGMISLIAALAVDYVIG | |
| | MENAMPWNLPADLAWFKRNTLNKPVIMGRHTWESIG | |
| | RPLPGRKNIILSSQPSTDDRVTWVKSVDEAIAACGDVPE | |
| | IMVIGGGRVIEQFLPKAQKLYLTHIDAEVEGDTHFPDYE | |
| | PDDWESVFSEFHDADAQNSHSYCFEILERRGSGQCTNY | |
| | ALLKLAGDVESNPGPLEMALPVTALLLPLALLLHAARP | |
| | SQFRVSPLDRTWNLGETVELKCQVLLSNPTSGCSWLFQ | |
| | PRGAAASPTFLLYLSQNKPKAAEGLDTQRFSGKRLGDT | |
| | FVLTLSDFRRENEGYYFCSALSNSIMYFSHFVPVFLPAK | |
| | PTTTPAPRPPTPAPTIASQPLSLRPEACRPAAGGAVHTRG | |
| | LDFACDIYIWAPLAGTCGVLLLSLVITLYCNHRNRRRV | |
| | CKCPRPVVKSGDKPSLSARYV | |
| Vmut3 3xHA DHFR-DD | MAGPVPRIIITKPEEEGPPVASGSGRSGYPYDVPDYAGY | 72 |
| N type hCD8 | PYDVPDYAGYPYDVPDYAGSGMISLIAALAVDYVIGM | |
| | ENAMPWNLPADLAWFKRNTLNKPVIMGRHTWESIGRP | |
| | LPGRKNIILSSQPSTDDRVTWVKSVDEAIAACGDVPEIM | |
| | VIGGGRVIEQFLPKAQKLYLTHIDAEVEGDTHFPDYEPD | |
| | DWESVFSEFHDADAQNSHSYCFEILERRGSGQCTNYAL | |
| | LKLAGDVESNPGPLEMALPVTALLLPLALLLHAARPSQ | |
| | FRVSPLDRTWNLGETVELKCQVLLSNPTSGCSWLFQPR | |
| | GAAASPTFLLYLSQNKPKAAEGLDTQRFSGKRLGDTFV | |
| | LTLSDFRRENEGYYFCSALSNSIMYFSHFVPVFLPAKPT | |
| | TTPAPRPPTPAPTIASQPLSLRPEACRPAAGGAVHTRGL | |
| | DFACDIYIWAPLAGTCGVLLLSLVITLYCNHRNRRRVC | |
| | KCPRPVVKSGDKPSLSARYV | |
| VIVIT GSTA1 3xHA | MAGPHPVIVITGPHEEGPPVASGSGRSGMAEKPKLHYF | 73 |
| hCD8 | NARGRMESTRWLLAAAGVEFEEKFIKSAEDLDKLRND | |
| | GYLMFQQVPMVEIDGMKLVQTRAILNYIASKYNLYGK | |
| | DIKERALIDMYIEGIADLGEMILLLPVCPPEEKDAKLALI | |
| | KEKIKNRYFPAFEKVLKSHGQDYLVGNKLSRADIHLVE | |
| | LLYYVEELDSSLISSFPLLKALKTRISNLPTVKKFLQPGS | |
| | PRKPPMDEKSLEEARKIFRFGTGYPYDVPDYAGYPYDV | |
| | PDYAGYPYDVPDYAALGQCTNYALLKLAGDVESNPGP | |
| | LEMALPVTALLLPLALLLHAARPSQFRVSPLDRTWNLG | |
| | ETVELKCQVLLSNPTSGCSWLFQPRGAAASPTFLLYLSQ | |
| | NKPKAAEGLDTQRFSGKRLGDTFVLTLSDFRRENEGYY | |
| | FCSALSNSIMYFSHFVPVFLPAKPTTTPAPRPPTPAPTIAS | |
| | QPLSLRPEACRPAAGGAVHTRGLDFACDIYIWAPLAGT | |
| | CGVLLLSLVITLYCNHRNRRRVCKCPRPVVKSGDKPSL | |
| | SARYV | |
| Vmut3 GSTA1 3xHA | MAGPVPRIIITKPEEEGPPVASGSGRSGMAEKPKLHYFN | 74 |
| hCD8 | ARGRMESTRWLLAAAGVEFEEKFIKSAEDLDKLRNDG | |
| | YLMFQQVPMVEIDGMKLVQTRAILNYIASKYNLYGKDI | |
| | KERALIDMYIEGIADLGEMILLLPVCPPEEKDAKLALIKE | |
| | KIKNRYFPAFEKVLKSHGQDYLVGNKLSRADIHLVELL | |
| | YYVEELDSSLISSFPLLKALKTRISNLPTVKKFLQPGSPR | |
| | KPPMDEKSLEEARKIFRFGTGYPYDVPDYAGYPYDVPD | |
| | YAGYPYDVPDYAALGQCTNYALLKLAGDVESNPGPLE | |
| | MALPVTALLLPLALLLHAARPSQFRVSPLDRTWNLGET | |
| | VELKCQVLLSNPTSGCSWLFQPRGAAASPTFLLYLSQN | |
| | KPKAAEGLDTQRFSGKRLGDTFVLTLSDFRRENEGYYF | |
| | CSALSNSIMYFSHFVPVFLPAKPTTTPAPRPPTPAPTIAS | |
| | QPLSLRPEACRPAAGGAVHTRGLDFACDIYIWAPLAGT | |
| | CGVLLLSLVITLYCNHRNRRRVCKCPRPVVKSGDKPSL | |
| | SARYV | |
| VIVIT GFP-Vmut1 | MAGPVPRIIITKPYEEGPPVASGSGRSGMVSKGEELFTG | 75 |
| | VVPILVELDGDVNGHKFSVSGEGEGDATYGKLTLKFIC | |
| | TTGKLPVPWPTLVTTLTYGVQCFSRYPDHMKQHDFFKS | |
| | AMPEGYVQERTIFFKDDGNYKTRAEVKFEGDTLVNRIE | |
| | LKGIDFKEDGNILGHKLEYNYNSHNVYIMADKQKNGIK | |
| | VNFKIRHNIEDGSVQLADHYQQNTPIGDGPVLLPDNHY | |
| | LSTQSALSKDPNEKRDHMVLLEFVTAAGITLGMDELYK | |
| VIVIT GFP-Vmut2 | MAGPVPRIIITKPDEEGPPVASGSGRSGMVSKGEELFTG | 76 |
| | VVPILVELDGDVNGHKFSVSGEGEGDATYGKLTLKFIC | |
| | TTGKLPVPWPTLVTTLTYGVQCFSRYPDHMKQHDFFKS | |
| | AMPEGYVQERTIFFKDDGNYKTRAEVKFEGDTLVNRIE | |
| | LKGIDFKEDGNILGHKLEYNYNSHNVYIMADKQKNGIK | |
| | VNFKIRHNIEDGSVQLADHYQQNTPIGDGPVLLPDNHY | |
| | LSTQSALSKDPNEKRDHMVLLEFVTAAGITLGMDELYK | |
| VIVIT GFP-Vmut3 | MAGPVPRIIITKPEEEGPPVASGSGRSGMVSKGEELFTG | 77 |
| | VVPILVELDGDVNGHKFSVSGEGEGDATYGKLTLKFIC | |
| | TTGKLPVPWPTLVTTLTYGVQCFSRYPDHMKQHDFFKS | |
| | AMPEGYVQERTIFFKDDGNYKTRAEVKFEGDTLVNRIE | |
| | LKGIDFKEDGNILGHKLEYNYNSHNVYIMADKQKNGIK | |
| | VNFKIRHNIEDGSVQLADHYQQNTPIGDGPVLLPDNHY | |
| | LSTQSALSKDPNEKRDHMVLLEFVTAAGITLGMDELYK | |
| VIVIT GFP-Vmut5 | MASGLLPRIIITPPYELGPPVASGSGRSGMVSKGEELFTG | 78 |
| | VVPILVELDGDVNGHKFSVSGEGEGDATYGKLTLKFIC | |
| | TTGKLPVPWPTLVTTLTYGVQCFSRYPDHMKQHDFFKS | |
| | AMPEGYVQERTIFFKDDGNYKTRAEVKFEGDTLVNRIE | |
| | LKGIDFKEDGNILGHKLEYNYNSHNVYIMADKQKNGIK | |
| | VNFKIRHNIEDGSVQLADHYQQNTPIGDGPVLLPDNHY | |
| | LSTQSALSKDPNEKRDHMVLLEFVTAAGITLGMDELYK | |
| VIVIT GFP NFAT SFFV | CACACAAAAAACCAACACACAGATGTAATGAAAATA | 79 |
| BFP | AAGATATTTTATTTAAGCTTTActtgtacagctcgtccatgccgaga | |
| | gtgatcccggcggcggtcacgaactccagcaggaccatgtgatcgcgcttctcgttgg | |
| | ggtctttgctcagggcggactgggtgctcaggtagtggttgtcgggcagcagcacggg | |
| | gccgtcgccgatgggggtgttctgctggtagtggtcggcgagctgcacgctgccgtcct | |
| | cgatgttgtggcggatcttgaagttcaccttgatgccgttcttctgcttgtcggccatga | |
| | tatagacgttgtggctgttgtagttgtactccagcttgtgccccaggatgttgccgtcct | |
| | ccttgaagtcgatgcccttcagctcgatgcggttcaccagggtgtcgccctcgaacttca | |
| | cctcggcgcgggtcttgtagttgccgtcgtccttgaagaagatggtgcgctcctggacgt | |
| | agccttcgggcatggcggacttgaagaagtcgtgctgcttcatgtggtcggggtagcggc | |
| | tgaagcactgcacgccgtaggtcagggtggtcacgaggggggccagggcacgggcagct | |
| | tgccggtggtgcagatgaacttcagggtcagcttgccgtaggtggcategccctcgccc | |
| | tcgccggacacgctgaacttgtggccgtttacgtcgccgtccagctcgaccaggatgg | |
| | gcaccaccccggtgaacagctcctcgcccttgctcaccatCCCAGATCTtcctg | |
| | atccgctTgcgacagggggcccctcctcgtgtggaccTgtaattacgattactgggtgt | |
| | ggtcccgccatGGTACGCGTCAGGAGTTGAGGTTACTGTG | |
| | AGTAGTGATTAAAGAGAGTGATAGGGAACTCTTGAA | |
| | CAAGAGATGCAATTTATACTGTTAATTCTGGAAAAAT | |
| | ATTATGGGGGTGTCAAAATGTCCCGGGACCAATTGA | |
| | CGCCTTCTGTATGAAACAGTTTTTCCTCCTAATTGAC | |
| | GCCTTCTGTATGAAACAGTTTTTCCTCCTAATTGACG | |
| | CCTTCTGTATGAAACAGTTTTTCCTCCTAATTCGATG | |
| | GGACCAATTGACGCCTTCTGTATGAAACAGTTTTTCC | |
| | TCCTAATTGACGCCTTCTGTATGAAACAGTTTTTCCTC | |
| | CTAATTGACGCCTTCTGTATGAAACAGTTTTTCCTCCT | |
| | AATTCGATATCAAGCTGGCGGCCGCAACTGGCCGGT | |
| | ACCTGTAACGCCATTTTGCAAGGCATGGAAAAATAC | |
| | CAAACCAAGAATAGAGAAGTTCAGATCAAGGGCGGG | |
| | TACATGAAAATAGCTAACGTTGGGCCAAACAGGATA | |
| | TCTGCGGTGAGCAGTTTCGGCCCCGGCCCGGGGCCA | |
| | AGAACAGATGGTCACCGCAGTTTCGGCCCCGGCCCG | |
| | AGGCCAAGAACAGATGGTCCCCAGATATGGCCCAAC | |
| | CCTCAGCAGTTTCTTAAGACCCATCAGATGTTTCCAG | |
| | GCTCCCCCAAGGACCTGAAATGACCCTGCGCCTTATT | |
| | TGAATTAACCAATCAGCCTGCTTCTCGCTTCTGTTCG | |
| | CGCGCTTCTGCTTCCCGAGCTCTATAAAAGAGCTCAC | |
| | AACCCCTCACTCGGCGCGCCAGTCCTCCGACAGACTG | |
| | AGTCGGGATCCACCatgagcgagctgattaaggagaacatgcacatgaag | |
| | ctgtacatggagggcaccgtggacaaccatcacttcaagtgcacatccgagggcgaag | |
| | gcaagccctacgagggcacccagaccatgagaatcaaggtggtcgagggcggccct | |
| | ctccccttcgccttegacatcctggctactagcttcctctacggcagcaagaccttcatc | |
| | aaccacacccagggcatccccgacTTTttcaagcagtccttccctgagggcttcacatg | |
| | ggagagagtcaccacatacgaagacgggggcgtgctgaccgctacccaggacacca | |
| | gcctccaggacggctgcctcatctacaacgtcaagatcagaggggtgaacTTTacat | |
| | ccaacggccctgtgatgcagaagaaaacactcggctgggaggccttcaccgagacgc | |
| | tgtaccccgctgacggcggcctggaaggcagaaacgacatggccctgaagctcgtgg | |
| | gcgggagccatctgatcgcaaacatcaagaccacatatagatccaagaaacccgctaa | |
| | gaacctcaagatgcctggcgtctactatgtggactacagactggaaagaatcaaggag | |
| | gccaacaacgagacctacgtcgagcagcacgaggtggcagtggccagatactgcga | |
| | cctccctagcaaactgggCcacaagctaaat | |
| VIVIT GFP NFKB SFFV | CACACAAAAAACCAACACACAGATGTAATGAAAATA | 80 |
| BFP | AAGATATTTTATTTAAGCTTTActtgtacagctcgtccatgccgaga | |
| | gtgatcccggcggcggtcacgaactccagcaggaccatgtgatcgcgcttctcgttgg | |
| | ggtctttgctcagggcggactgggtgctcaggtagtggttgtcgggcagcagcacggg | |
| | gccgtcgccgatgggggtgttctgctggtagtggtcggcgagctgcacgctgccgtcct | |
| | cgatgttgtggcggatcttgaagttcaccttgatgccgttcttctgcttgtcggccatga | |
| | tatagacgttgtggctgttgtagttgtactccagcttgtgccccaggatgttgccgtcct | |
| | ccttgaagtcgatgcccttcagctcgatgcggttcaccagggtgtcgccctegaacttca | |
| | cctcggcgcgggtcttgtagttgccgtcgtccttgaagaagatggtgcgctcctggacgt | |
| | agccttcgggcatggcggacttgaagaagtcgtgctgcttcatgtggtcggggtagcggc | |
| | tgaagcactgcacgccgtaggtcagggtggtcacgagggtgggccagggcacgggcagct | |
| | tgccggtggtgcagatgaacttcagggtcagcttgccgtaggtggcategccctcgccc | |
| | tcgccggacacgctgaacttgtggccgtttacgtcgccgtccagctcgaccaggatgg | |
| | gcaccaccccggtgaacagctcctcgcccttgctcaccatCCCAGATCTtcctg | |
| | atccgctTgcgacagggggcccctcctcgtgtggaccTgtaattacgattactgggtgt | |
| | ggtcccgccatGGTAGGATCTAAGCTTcaacagtaccggaatgccaag | |
| | ctggaattcgagcttccattatataccctctagagtctccgcggaaagtccccagcggaa | |
| | agtccccagcggaaagtccccageggaaagtccccaagcggaaagtcccctagcttg | |
| | gatccagacatgataagaGAGCTCAGGTACCTGTAACGCCATT | |
| | TTGCAAGGCATGGAAAAATACCAAACCAAGAATAGA | |
| | GAAGTTCAGATCAAGGGCGGGTACATGAAAATAGCT | |
| | AACGTTGGGCCAAACAGGATATCTGCGGTGAGCAGT | |
| | TTCGGCCCCGGCCCGGGGCCAAGAACAGATGGTCAC | |
| | CGCAGTTTCGGCCCCGGCCCGAGGCCAAGAACAGAT | |
| | GGTCCCCAGATATGGCCCAACCCTCAGCAGTTTCTTA | |
| | AGACCCATCAGATGTTTCCAGGCTCCCCCAAGGACCT | |
| | GAAATGACCCTGCGCCTTATTTGAATTAACCAATCAG | |
| | CCTGCTTCTCGCTTCTGTTCGCGCGCTTCTGCTTCCCG | |
| | AGCTCTATAAAAGAGCTCACAACCCCTCACTCGGCG | |
| | CGCCAGTCCTCCGACAGACTGAGTCGGGATCCACCatg | |
| | agcgagctgattaaggagaacatgcacatgaagctgtacatggagggcaccgtggac | |
| | aaccatcacttcaagtgcacatccgagggcgaaggcaagccctacgagggcacccag | |
| | accatgagaatcaaggtggtcgagggcggccctctccccttcgccttcgacatcctggc | |
| | tactagcttcctctacggcagcaagaccttcatcaaccacacccagggcatccccgacT | |
| | TTttcaagcagtccttccctgagggcttcacatgggagagagtcaccacatacgaaga | |
| | cgggggcgtgctgaccgctacccaggacaccagcctccaggacggctgcctcatcta | |
| | caacgtcaagatcagaggggtgaacTTTacatccaacggccctgtgatgcagaaga | |
| | aaacactcggctgggaggccttcaccgagacgctgtaccccgctgacggcggcctgg | |
| | aaggcagaaacgacatggccctgaagctcgtgggcgggagccatctgatcgcaaaca | |
| | tcaagaccacatatagatccaagaaacccgctaagaacctcaagatgcctggcgtctac | |
| | tatgtggactacagactggaaagaatcaaggaggccaacaacgagacctacgtcgag | |
| | cagcacgaggtggcagtggccagatactgcgacctccctagcaaactgggCcacaa | |
| | gctaaat | |
| R2 3N PD-1EC CD28 | METDTLLLWVLLLWVPGSTGCPYSNPSLCSGGGGSSGG | 81 |
| delta Vmut3 P2A CD20 | GGSDCPYSNPSLCGGGGSGGGSLEIEAAFLRQENTALRT | |
| CD28EC CD28z | RVAELEQEVQRLENEVSQYETRYGPLGGGKGGGGSGG | |
| | GGSGGGGSGGGSLEIEAAFLERENTALETRVAELRQRV | |
| | QRLRNRVSQYRTRYGPLGGGKGGGGSGGGSTERILETS | |
| | TRYPSPSPKPEGRFQGMFWALVVVAGVLFCYGLLVTV | |
| | ALCVIWTRAKFSRSAETAANLQDTFDALHMQTLAPRG | |
| | GGGSGGGGSGGGGSTSMAGPVPRIIITKPEEEGSGTGRA | |
| | KRGSGATNFSLLKQAGDVEENPGPGSLTMASPLTRFLS | |
| | LNLLLLGESIILGSGEAQIVMSQSPAILSASPGEKVTMTC | |
| | RARSSVSYIHWYQQKPGSSPKPWIYATSNLASGVPGRF | |
| | SGSGSGTSYSLTITRVEAEDAATYYCQQWSSKPPTFGG | |
| | GTKLEIKRGGGGSGGGGSGGGGSQVQLQQPGAELVRP | |
| | GTSVKLSCKASGYTFTSYWMHWIKQRPGQGLEWIGVI | |
| | DPSDNYTKYNQKFKGKATLTVDTSSSTAYMQLSSLTSE | |
| | DSAVYFCAREGYYGSSPWFAYWGQGTLVTVSSSAWSH | |
| | PQFEKGGGSGGGSGGSAWSHPQFEKGGGGSGGGSEAR | |
| | LIEFMYPPPYLDNERSNGTIIHIKEKHLCHTQSSPKLFWA | |
| | LVVVAGVLFCYGLLVTVALCVIWTNSRRNRLLQSDYM | |
| | NMTPRRPGLTRKPYQPYAPARDFAAYRPRAKFSRSAET | |
| | AANLQDPNQLYNELNLGRREEYDVLEKKRARDPEMGG | |
| | KQQRRRNPQEGVYNALQKDKMAEAYSEIGTKGERRRG | |
| | KGHDGLYQGLSTATKDTYDALHMQTLAPRGSGQCTN | |
| | YALLKLAGDVESNPGPLE | |
| R2 3N PD-1EC CD28 | METDTLLLWVLLLWVPGSTGCPYSNPSLCSGGGGSSGG | 82 |
| delta Vmut3 3x P2A CD20 | GGSDCPYSNPSLCGGGGSGGGSLEIEAAFLRQENTALRT | |
| CD28EC CD28z | RVAELEQEVQRLENEVSQYETRYGPLGGGKGGGGSGG | |
| | GGSGGGGSGGGSLEIEAAFLERENTALETRVAELRQRV | |
| | QRLRNRVSQYRTRYGPLGGGKGGGGSGGGSTERILETS | |
| | TRYPSPSPKPEGRFQGMFWALVVVAGVLFCYGLLVTV | |
| | ALCVIWTRAKFSRSAETAANLQDTFDALHMQTLAPRG | |
| | GGGSGGGGSGGGGSTSMAGPVPRIIITKPEEEGSTGGGG | |
| | SGGGGSGGGGSMAGPVPRIIITKPEEEGSTGGGGSGGGG | |
| | SGGGGSMAGPVPRIIITKPEEEGSGTGRAKRGSGATNFS | |
| | LLKQAGDVEENPGPGSLTMASPLTRFLSLNLLLLGESIIL | |
| | GSGEAQIVMSQSPAILSASPGEKVTMTCRARSSVSYIHW | |
| | YQQKPGSSPKPWIYATSNLASGVPGRFSGSGSGTSYSLT | |
| | ITRVEAEDAATYYCQQWSSKPPTFGGGTKLEIKRGGGG | |
| | SGGGGSGGGGSQVQLQQPGAELVRPGTSVKLSCKASG | |
| | YTFTSYWMHWIKQRPGQGLEWIGVIDPSDNYTKYNQK | |
| | FKGKATLTVDTSSSTAYMQLSSLTSEDSAVYFCAREGY | |
| | YGSSPWFAYWGQGTLVTVSSSAWSHPQFEKGGGSGGG | |
| | SGGSAWSHPQFEKGGGGSGGGSEARLIEFMYPPPYLDN | |
| | ERSNGTIIHIKEKHLCHTQSSPKLFWALVVVAGVLFCYG | |
| | LLVTVALCVIWTNSRRNRLLQSDYMNMTPRRPGLTRK | |
| | PYQPYAPARDFAAYRPRAKFSRSAETAANLQDPNQLY | |
| | NELNLGRREEYDVLEKKRARDPEMGGKQQRRRNPQEG | |
| | VYNALQKDKMAEAYSEIGTKGERRRGKGHDGLYQGLS | |
| | TATKDTYDALHMQTLAPRGSGQCTNYALLKLAGDVES | |
| | NPGPLE | |
| R2 3N PD-1EC CD28 | METDTLLLWVLLLWVPGSTGCPYSNPSLCSGGGGSSGG | 83 |
| delta DAMP P2A CD20 | GGSDCPYSNPSLCGGGGSGGGSLEIEAAFLRQENTALRT | |
| CD28EC CD28z | RVAELEQEVQRLENEVSQYETRYGPLGGGKGGGGSGG | |
| | GGSGGGGSGGGSLEIEAAFLERENTALETRVAELRQRV | |
| | QRLRNRVSQYRTRYGPLGGGKGGGGSGGGSTERILETS | |
| | TRYPSPSPKPEGRFQGMFWALVVVAGVLFCYGLLVTV | |
| | ALCVIWTRAKFSRSAETAANLQDTFDALHMQTLAPRG | |
| | GGGSGGGGSGGGGSTSITSFEEAKGLDRINERMPPRRD | |
| | AMPGSGTGRAKRGSGATNFSLLKQAGDVEENPGPGSL | |
| | TMASPLTRFLSLNLLLLGESIILGSGEAQIVMSQSPAILSA | |
| | SPGEKVTMTCRARSSVSYIHWYQQKPGSSPKPWIYATS | |
| | NLASGVPGRFSGSGSGTSYSLTITRVEAEDAATYYCQQ | |
| | WSSKPPTFGGGTKLEIKRGGGGSGGGGSGGGGSQVQL | |
| | QQPGAELVRPGTSVKLSCKASGYTFTSYWMHWIKQRP | |
| | GQGLEWIGVIDPSDNYTKYNQKFKGKATLTVDTSSSTA | |
| | YMQLSSLTSEDSAVYFCAREGYYGSSPWFAYWGQGTL | |
| | VTVSSSAWSHPQFEKGGGSGGGSGGSAWSHPQFEKGG | |
| | GGSGGGSEARLIEFMYPPPYLDNERSNGTIIHIKEKHLC | |
| | HTQSSPKLFWALVVVAGVLFCYGLLVTVALCVIWINS | |
| | RRNRLLQSDYMNMTPRRPGLTRKPYQPYAPARDFAAY | |
| | RPRAKFSRSAETAANLQDPNQLYNELNLGRREEYDVLE | |
| | KKRARDPEMGGKQQRRRNPQEGVYNALQKDKMAEA | |
| | YSEIGTKGERRRGKGHDGLYQGLSTATKDTYDALHMQ | |
| | TLAPRGSGQCTNYALLKLAGDVESNPGPLE | |
| EGFP-DAMP | MVSKGEELFTGVVPILVELDGDVNGHKFSVSGEGEGDA | 84 |
| | TYGKLTLKFICTTGKLPVPWPTLVTTLTYGVQCFSRYPD | |
| | HMKQHDFFKSAMPEGYVQERTIFFKDDGNYKTRAEVK | |
| | FEGDTLVNRIELKGIDFKEDGNILGHKLEYNYNSHNVYI | |
| | MADKQKNGIKVNFKIRHNIEDGSVQLADHYQQNTPIGD | |
| | GPVLLPDNHYLSTQSALSKDPNEKRDHMVLLEFVTAA | |
| | GITLGMDELYKGGGGSGGGSGSLITSFEEAKGLDRINER | |
| | MPPRRDAMP | |
| Q2 RR12EE345L 2A iC9 | METDTLLLWVLLLWVPGSTGGSGELPTQGTFSNVSTNV | 85 |
| CD19 CD8EC CD28z | SGGGGSGGGSELPTQGTFSNVSTNVSGGGGSGGGSALG | |
| | LEIRAAFLRQRNTALRTEVAELEQEVQRLENEVSQYET | |
| | RYGPLGGGKGSGRAKRATNFSLLKQAGDVEENPGPEFT | |
| | RAKRMGVQVETISPGDGRTFPKRGQTCVVHYTGMLED | |
| | GKKVDSSRDRNKPFKFMLGKQEVIRGWEEGVAQMSV | |
| | GQRAKLTISPDYAYGATGHPGIIPPHATLVFDVELLKLE | |
| | SGGGSGVDGFGDVGALESLRGNADLAYILSMEPCGHC | |
| | LIINNVNFCRESGLRTRTGSNIDCEKLRRRFSSLHFMVE | |
| | VKGDLTAKKMVLALLELARQDHGALDCCVVVILSHGC | |
| | QASHLQFPGAVYGTDGCPVSVEKIVNIFNGTSCPSLGGK | |
| | PKLFFIQACGGEQKDHGFEVASTSPEDESPGSNPEPDAT | |
| | PFQEGLRTFDQLDAISSLPTPSDIFVSYSTFPGFVSWRDP | |
| | KSGSWYVETLDDIFEQWAHSEDLQSLLLRVANAVSVK | |
| | GIYKQMPGCFNFLRKKLFFKTSVDGTGRAKRVKQTLNF | |
| | DLLKLAGDVESNPGPGSLTMASPLTRFLSLNLLLLGESII | |
| | LGSGEAEVQLQQSGAELVRPGTSVKLSCKVSGDTITFY | |
| | YMHFVKQRPGQGLEWIGRIDPEDESTKYSEKFKNKATL | |
| | TADTSSNTAYLKLSSLTSEDTATYFCIYGGYYFDYWGQ | |
| | GVMVTVSSGGGGSGGGGSGGGGSDIQMTQSPASLSTSL | |
| | GETVTIQCQASEDIYSGLAWYQQKPGKSPQLLIYGASD | |
| | LQDGVPSRFSGSGSGTQYSLKITSMQTEDEGVYFCQQG | |
| | LTYPRTFGGGTKLELKREQKLISEEDLLESSTTTKPVLRT | |
| | PSPVHPTGTSQPQRPEDCRPRGSVKGTGLDFACDIYIWA | |
| | PLAGICVALLLSLIITLICYNSRRNRLLQSDYMNMTPRRP | |
| | GLTRKPYQPYAPARDFAAYRPRAKFSRSAETAANLQDP | |
| | NQLYNELNLGRREEYDVLEKKRARDPEMGGKQQRRR | |
| | NPQEGVYNALQKDKMAEAYSEIGTKGERRRGKGHDGL | |
| | YQGLSTATKDTYDALHMQTLAPRGTEARL | |
| FLAG-RR12EE345L P2A | METDTLLLWVLLLWVPGSTGDYKDDDDKLEIRAAFLR | 86 |
| iC9 F2A CD19 CD8H | QRNTALRTEVAELEQEVQRLENEVSQYETRYGPLGGG | |
| CD8TM BBz | KGSGRAKRATNFSLLKQAGDVEENPGPEFTRAKRMGV | |
| | QVETISPGDGRTFPKRGQTCVVHYTGMLEDGKKVDSSR | |
| | DRNKPFKFMLGKQEVIRGWEEGVAQMSVGQRAKLTIS | |
| | PDYAYGATGHPGIIPPHATLVFDVELLKLESGGGSGVD | |
| | GFGDVGALESLRGNADLAYILSMEPCGHCLIINNVNFC | |
| | RESGLRTRTGSNIDCEKLRRRFSSLHFMVEVKGDLTAK | |
| | KMVLALLELARQDHGALDCCVVVILSHGCQASHLQFP | |
| | GAVYGTDGCPVSVEKIVNIFNGTSCPSLGGKPKLFFIQA | |
| | CGGEQKDHGFEVASTSPEDESPGSNPEPDATPFQEGLRT | |
| | FDQLDAISSLPTPSDIFVSYSTFPGFVSWRDPKSGSWYV | |
| | ETLDDIFEQWAHSEDLQSLLLRVANAVSVKGIYKQMPG | |
| | CFNFLRKKLFFKTSVDGTGRAKRVKQTLNFDLLKLAGD | |
| | VESNPGPGSLTMASPLTRFLSLNLLLLGESIILGSGEAEV | |
| | QLQQSGAELVRPGTSVKLSCKVSGDTITFYYMHFVKQR | |
| | PGQGLEWIGRIDPEDESTKYSEKFKNKATLTADTSSNTA | |
| | YLKLSSLTSEDTATYFCIYGGYYFDYWGQGVMVTVSS | |
| | GGGGSGGGGSGGGGSDIQMTQSPASLSTSLGETVTIQC | |
| | QASEDIYSGLAWYQQKPGKSPQLLIYGASDLQDGVPSR | |
| | FSGSGSGTQYSLKITSMQTEDEGVYFCQQGLTYPRTFG | |
| | GGTKLELKREQKLISEEDLLESSTTTKPVLRTPSPVHPTG | |
| | TSQPQRPEDCRPRGSVKGTGLDFACDIYIWAPLAGICVA | |
| | LLLSLIITLICYKRGRKKLLYIFKQPFMRPVQTTQEEDGC | |
| | SCRFPEEEEGGCELRAKFSRSAETAANLQDPNQLYNEL | |
| | NLGRREEYDVLEKKRARDPEMGGKQQRRRNPQEGVY | |
| | NALQKDKMAEAYSEIGTKGERRRGKGHDGLYQGLSTA | |
| | TKDTYDALHMQTLAPRGTEARL | |
| RR12EE345L linker | METDTLLLWVLLLWVPGSTGLEIRAAFLRQRNTALRTE | 87 |
| EE12RR345L Thy1.1 P2A | VAELEQEVQRLENEVSQYETRYGPLGGGKGGGGSGGG | |
| CD20 CD28H CD28TM | GSGGGGSGGGSLEIEAAFLERENTALETRVAELRQRVQ | |
| BBz E2A | RLRNRVSQYRTRYGPLGGGKEFTQKVTSLTACLVNQN | |
| | LRLDCRHENNTKDNSIQHEFSLTREKRKHVLSGTLGIPE | |
| | HTYRSRVTLSNQPYIKVLTLANFTTKDEGDYFCELRVS | |
| | GANPMSSNKSISVYRDKLVKCGGISLLVQNTSWMLLLL | |
| | LSLSLLQALDFISLGSGATNFSLLKQAGDVEENPGPGSL | |
| | TMASPLTRFLSLNLLLLGESIILGSGEAQIVMSQSPAILSA | |
| | SPGEKVTMTCRARSSVSYIHWYQQKPGSSPKPWIYATS | |
| | NLASGVPGRFSGSGSGTSYSLTITRVEAEDAATYYCQQ | |
| | WSSKPPTFGGGTKLEIKRGGGGSGGGGSGGGGSQVQL | |
| | QQPGAELVRPGTSVKLSCKASGYTFTSYWMHWIKQRP | |
| | GQGLEWIGVIDPSDNYTKYNQKFKGKATLTVDTSSSTA | |
| | YMQLSSLTSEDSAVYFCAREGYYGSSPWFAYWGQGTL | |
| | VTVSSSAWSHPQFEKGGGSGGGSGGSAWSHPQFEKGG | |
| | GGSGGGSEARLIEFMYPPPYLDNERSNGTIIHIKEKHLC | |
| | HTQSSPKLFWALVVVAGVLFCYGLLVTVALCVIWTKR | |
| | GRKKLLYIFKQPFMRPVQTTQEEDGCSCRFPEEEEGGCE | |
| | LRAKFSRSAETAANLQDPNQLYNELNLGRREEYDVLEK | |
| | KRARDPEMGGKQQRRRNPQEGVYNALQKDKMAEAYS | |
| | EIGTKGERRRGKGHDGLYQGLSTATKDTYDALHMQTL | |
| | APRGSGQCTNYALLKLAGDVESNPGP | |
| FLAG-RR12EE345L P2A | METDTLLLWVLLLWVPGSTGDYKDDDDKLEIRAAFLR | 88 |
| iC9 F2A 3xHA NFKBIAm | QRNTALRTEVAELEQEVQRLENEVSQYETRYGPLGGG | |
| E2A CD19 CD8H CD8TM | KGSGRAKRATNFSLLKQAGDVEENPGPEFTRAKRMGV | |
| BBz | QVETISPGDGRTFPKRGQTCVVHYTGMLEDGKKVDSSR | |
| | DRNKPFKFMLGKQEVIRGWEEGVAQMSVGQRAKLTIS | |
| | PDYAYGATGHPGIIPPHATLVFDVELLKLESGGGSGVD | |
| | GFGDVGALESLRGNADLAYILSMEPCGHCLIINNVNFC | |
| | RESGLRTRTGSNIDCEKLRRRFSSLHFMVEVKGDLTAK | |
| | KMVLALLELARQDHGALDCCVVVILSHGCQASHLQFP | |
| | GAVYGTDGCPVSVEKIVNIFNGTSCPSLGGKPKLFFIQA | |
| | CGGEQKDHGFEVASTSPEDESPGSNPEPDATPFQEGLRT | |
| | FDQLDAISSLPTPSDIFVSYSTFPGFVSWRDPKSGSWYV | |
| | ETLDDIFEQWAHSEDLQSLLLRVANAVSVKGIYKQMPG | |
| | CFNFLRKKLFFKTSVDGTGRAKRVKQTLNFDLLKLAGD | |
| | VESNPGPGSGYPYDVPDYAGYPYDVPDYAGYPYDVPD | |
| | YAGSGMFQPAGHGQDWAMEGPRDGLKKERLVDDRH | |
| | DAGLDAMKDEEYEQMVKELREIRLQPQEAPLAAEPWK | |
| | QQLTEDGDSFLHLAIIHEEKPLTMEVIGQVKGDLAFLNF | |
| | QNNLQQTPLHLAVITNQPGIAEALLKAGCDPELRDFRG | |
| | NTPLHLACEQGCLASVAVLTQTCTPQHLHSVLQATNY | |
| | NGHTCLHLASIHGYLAIVEHLVTLGADVNAQEPCNGRT | |
| | ALHLAVDLQNPDLVSLLLKCGADVNRVTYQGYSPYQL | |
| | TWGRPSTRIQQQLGQLTLENLQMLPEAEDEEAYDAEAE | |
| | FAEDELPDDDCVFGGQRLTLGSGRAKRQCTNYALLKL | |
| | AGDVESNPGPGSLTMASPLTRFLSLNLLLLGESIILGSGE | |
| | AEVQLQQSGAELVRPGTSVKLSCKVSGDTITFYYMHFV | |
| | KQRPGQGLEWIGRIDPEDESTKYSEKFKNKATLTADTSS | |
| | NTAYLKLSSLTSEDTATYFCIYGGYYFDYWGQGVMVT | |
| | VSSGGGGSGGGGSGGGGSDIQMTQSPASLSTSLGETVTI | |
| | QCQASEDIYSGLAWYQQKPGKSPQLLIYGASDLQDGVP | |
| | SRFSGSGSGTQYSLKITSMQTEDEGVYFCQQGLTYPRTF | |
| | GGGTKLELKREQKLISEEDLLESSTTTKPVLRTPSPVHPT | |
| | GTSQPQRPEDCRPRGSVKGTGLDFACDIYIWAPLAGICV | |
| | ALLLSLIITLICYKRGRKKLLYIFKQPFMRPVQTTQEEDG | |
| | CSCRFPEEEEGGCELRAKFSRSAETAANLQDPNQLYNE | |
| | LNLGRREEYDVLEKKRARDPEMGGKQQRRRNPQEGV | |
| | YNALQKDKMAEAYSEIGTKGERRRGKGHDGLYQGLST | |
| | ATKDTYDALHMQTLAPRGTEARL | |
| RR12EE345L linker | METDTLLLWVLLLWVPGSTGLEIRAAFLRQRNTALRTE | 89 |
| EE12RR345L Thy1.1 P2A | VAELEQEVQRLENEVSQYETRYGPLGGGKGGGGSGGG | |
| CD20 CD28H CD28TM | GSGGGGSGGGSLEIEAAFLERENTALETRVAELRQRVQ | |
| BBz E2A 3xHA | RLRNRVSQYRTRYGPLGGGKEFTQKVTSLTACLVNQN | |
| NFKBIAm | LRLDCRHENNTKDNSIQHEFSLTREKRKHVLSGTLGIPE | |
| | HTYRSRVTLSNQPYIKVLTLANFTTKDEGDYFCELRVS | |
| | GANPMSSNKSISVYRDKLVKCGGISLLVQNTSWMLLLL | |
| | LSLSLLQALDFISLGSGATNFSLLKQAGDVEENPGPGSL | |
| | TMASPLTRFLSLNLLLLGESIILGSGEAQIVMSQSPAILSA | |
| | SPGEKVTMTCRARSSVSYIHWYQQKPGSSPKPWIYATS | |
| | NLASGVPGRFSGSGSGTSYSLTITRVEAEDAATYYCQQ | |
| | WSSKPPTFGGGTKLEIKRGGGGSGGGGSGGGGSQVQL | |
| | QQPGAELVRPGTSVKLSCKASGYTFTSYWMHWIKQRP | |
| | GQGLEWIGVIDPSDNYTKYNQKFKGKATLTVDTSSSTA | |
| | YMQLSSLTSEDSAVYFCAREGYYGSSPWFAYWGQGTL | |
| | VTVSSSAWSHPQFEKGGGSGGGSGGSAWSHPQFEKGG | |
| | GGSGGGSEARLIEFMYPPPYLDNERSNGTIIHIKEKHLC | |
| | HTQSSPKLFWALVVVAGVLFCYGLLVTVALCVIWTKR | |
| | GRKKLLYIFKQPFMRPVQTTQEEDGCSCRFPEEEEGGCE | |
| | LRAKFSRSAETAANLQDPNQLYNELNLGRREEYDVLEK | |
| | KRARDPEMGGKQQRRRNPQEGVYNALQKDKMAEAYS | |
| | EIGTKGERRRGKGHDGLYQGLSTATKDTYDALHMQTL | |
| | APRGSGQCTNYALLKLAGDVESNPGPLEYPYDVPDYA | |
| | GYPYDVPDYAGYPYDVPDYAGSGMFQPAGHGQDWA | |
| | MEGPRDGLKKERLVDDRHDAGLDAMKDEEYEQMVKE | |
| | LREIRLQPQEAPLAAEPWKQQLTEDGDSFLHLAIIHEEK | |
| | PLTMEVIGQVKGDLAFLNFQNNLQQTPLHLAVITNQPGI | |
| | AEALLKAGCDPELRDFRGNTPLHLACEQGCLASVAVLT | |
| | QTCTPQHLHSVLQATNYNGHTCLHLASIHGYLAIVEHL | |
| | VTLGADVNAQEPCNGRTALHLAVDLQNPDLVSLLLKC | |
| | GADVNRVTYQGYSPYQLTWGRPSTRIQQQLGQLTLEN | |
| | LQMLPEAEDEEAYDAEAEFAEDELPDDDCVFGGQRLT | |
| | L | |
| RR12EE345L linker | METDTLLLWVLLLWVPGSTGLEIRAAFLRQRNTALRTE | 90 |
| EE12RR345L Thy1.1 | VAELEQEVQRLENEVSQYETRYGPLGGGKGGGGSGGG | |
| CD20 CD28EC BBz 3xHA | GSGGGGSGGGSLEIEAAFLERENTALETRVAELRQRVQ | |
| DHFR-DD NFKBIAm | RLRNRVSQYRTRYGPLGGGKEFTQKVTSLTACLVNQN | |
| | LRLDCRHENNTKDNSIQHEFSLTREKRKHVLSGTLGIPE | |
| | HTYRSRVTLSNQPYIKVLTLANFTTKDEGDYFCELRVS | |
| | GANPMSSNKSISVYRDKLVKCGGISLLVQNTSWMLLLL | |
| | LSLSLLQALDFISLGSGATNFSLLKQAGDVEENPGPGSL | |
| | TMASPLTRFLSLNLLLLGESIILGSGEAQIVMSQSPAILSA | |
| | SPGEKVTMTCRARSSVSYIHWYQQKPGSSPKPWIYATS | |
| | NLASGVPGRFSGSGSGTSYSLTITRVEAEDAATYYCQQ | |
| | WSSKPPTFGGGTKLEIKRGGGGSGGGGSGGGGSQVQL | |
| | QQPGAELVRPGTSVKLSCKASGYTFTSYWMHWIKQRP | |
| | GQGLEWIGVIDPSDNYTKYNQKFKGKATLTVDTSSSTA | |
| | YMQLSSLTSEDSAVYFCAREGYYGSSPWFAYWGQGTL | |
| | VTVSSSAWSHPQFEKGGGSGGGSGGSAWSHPQFEKGG | |
| | GGSGGGSEARLIEFMYPPPYLDNERSNGTIIHIKEKHLC | |
| | HTQSSPKLFWALVVVAGVLFCYGLLVTVALCVIWTKR | |
| | GRKKLLYIFKQPFMRPVQTTQEEDGCSCRFPEEEEGGCE | |
| | LRAKFSRSAETAANLQDPNQLYNELNLGRREEYDVLEK | |
| | KRARDPEMGGKQQRRRNPQEGVYNALQKDKMAEAYS | |
| | EIGTKGERRRGKGHDGLYQGLSTATKDTYDALHMQTL | |
| | APRGSGQCTNYALLKLAGDVESNPGPLEYPYDVPDYA | |
| | GYPYDVPDYAGYPYDVPDYAGSGMFQPAGHGQDWA | |
| | MEGPRDGLKKERLVDDRHDAGLDAMKDEEYEQMVKE | |
| | LREIRLQPQEAPLAAEPWKQQLTEDGDSFLHLAIIHEEK | |
| | PLTMEVIGQVKGDLAFLNFQNNLQQTPLHLAVITNQPGI | |
| | AEALLKAGCDPELRDFRGNTPLHLACEQGCLASVAVLT | |
| | QTCTPQHLHSVLQATNYNGHTCLHLASIHGYLAIVEHL | |
| | VTLGADVNAQEPCNGRTALHLAVDLQNPDLVSLLLKC | |
| | GADVNRVTYQGYSPYQLTWGRPSTRIQQQLGQLTLEN | |
| | LQMLPEAEDEEAYDAEAEFAEDELPDDDCVFGGQRLT | |
| | LGSGTRGSTGGGGSGGGGSGGGGSISLIAALAVDHVIG | |
| | METVMPWNLPADLAWFKRNTLNKPVIMGRHTWESIGR | |
| | PLPGRKNIILSSQPSTDDRVTWVKSVDEAIAACGDVPEI | |
| | MVIGGGRVYEQFLPKAQKLYLTHIDAEVEGDTHFPDYE | |
| | PDDWESVFSEFHDADAQNSHSYCFEILERR | |
| NFKBIAm NFAT SFFV | CACACAAAAAACCAACACACAGATGTAATGAAAATA | 91 |
| BFP | AAGATATTTTATTTAAGCTTTATTAtaatgtcagacgctggcctc | |
| | caaacacacagtcatcatcgggcagctcatcctctgcgaactctgcctccgcgtcatag | |
| | gcctcctcatcctcggcctcaggtagcatctggagattttccaaggtcagctggcccagc | |
| | tgctgctgtatccgggtacttgggcggccccaggtaagctggtagggggagtagccttg | |
| | gtaggttaccctgttgacatcagccccacatttcaacaagagcgaaaccaggtcaggatt | |
| | ctgcaggtccaccgcaaggtggagggctgtccggccattgcagggctcctgagcgttg | |
| | acatcagcacccaaagtcaccaagtgctccacgatggccaggtagccgtggatagag | |
| | gctaggtgcagacacgtgtggccattgtagttggtggcctgTaggacggagtggagat | |
| | gctggggtgtgcaggtctgcgtcaagactgctacactggccaggcagccctgctcaca | |
| | ggcaagatgtagaggggtatttcctcgaaagtctcggagTtcaggatcacagccagctt | |
| | tcagaagtgcctcagcaattcctggctggttggtgatcacagccaagtggagtggagtct | |
| | gctgcaggttgttctggaagttgaggaaggccaggtctcccttcacctgaccaatgactt | |
| | ccatTgtcagcggcttctcttcgtggatgattgccaagtgcaggaacgagtctccgtcct | |
| | ccgtgagctgctgcttccagggctcggcggccagcggcgcctcctgcggctgcaggc | |
| | ggatctcccgcagctccttcaccatttgctcgtactcctcgtccttcatggegtcAagac | |
| | ctgcgtcgtggcggtcgtccaccaagcgctccttcttgaggccatcccgcgggccctcca | |
| | tTgcccagtcctggccgtgcccagctggctgaaacattcctgatccagcAtaatcAg | |
| | gaacGtcgtaAgggtagccagcgtaatctggaacatcgtatggAtagccagcAtaa | |
| | tcGggaacatcAtaGggAtaCATGGTCCATGGACGCGTCAGG | |
| | AGTTGAGGTTACTGTGAGTAGTGATTAAAGAGAGTG | |
| | ATAGGGAACTCTTGAACAAGAGATGCAATTTATACT | |
| | GTTAATTCTGGAAAAATATTATGGGGGTGTCAAAAT | |
| | GTCCCGGGACCAATTGACGCCTTCTGTATGAAACAGT | |
| | TTTTCCTCCTAATTGACGCCTTCTGTATGAAACAGTTT | |
| | TTCCTCCTAATTGACGCCTTCTGTATGAAACAGTTTTT | |
| | CCTCCTAATTCGATGGGACCAATTGACGCCTTCTGTA | |
| | TGAAACAGTTTTTCCTCCTAATTGACGCCTTCTGTAT | |
| | GAAACAGTTTTTCCTCCTAATTGACGCCTTCTGTATG | |
| | AAACAGTTTTTCCTCCTAATTCGATATCAAGCTGGCG | |
| | GCCGCAACTGGCCGGTACCTGTAACGCCATTTTGCAA | |
| | GGCATGGAAAAATACCAAACCAAGAATAGAGAAGTT | |
| | CAGATCAAGGGCGGGTACATGAAAATAGCTAACGTT | |
| | GGGCCAAACAGGATATCTGCGGTGAGCAGTTTCGGC | |
| | CCCGGCCCGGGGCCAAGAACAGATGGTCACCGCAGT | |
| | TTCGGCCCCGGCCCGAGGCCAAGAACAGATGGTCCC | |
| | CAGATATGGCCCAACCCTCAGCAGTTTCTTAAGACCC | |
| | ATCAGATGTTTCCAGGCTCCCCCAAGGACCTGAAATG | |
| | ACCCTGCGCCTTATTTGAATTAACCAATCAGCCTGCT | |
| | TCTCGCTTCTGTTCGCGCGCTTCTGCTTCCCGAGCTCT | |
| | ATAAAAGAGCTCACAACCCCTCACTCGGCGCGCCAG | |
| | TCCTCCGACAGACTGAGTCGGGATCCACCatgagcgagctg | |
| | attaaggagaacatgcacatgaagctgtacatggagggcaccgtggacaaccatcactt | |
| | caagtgcacatccgagggcgaaggcaagccctacgagggcacccagaccatgagaa | |
| | tcaaggtggtcgagggcggccctctccccttcgccttcgacatcctggctactagcttcc | |
| | tctacggcagcaagaccttcatcaaccacacccagggcatccccgacTTTttcaagc | |
| | agtccttccctgagggcttcacatgggagagagtcaccacatacgaagacgggggcgt | |
| | gctgaccgctacccaggacaccagcctccaggacggctgcctcatctacaacgtcaag | |
| | atcagaggggtgaacTTTacatccaacggccctgtgatgcagaagaaaacactcgg | |
| | ctgggaggccttcaccgagacgctgtaccccgctgacggcggcctggaaggcagaa | |
| | acgacatggccctgaagctcgtggggggagccatctgatcgcaaacatcaagacca | |
| | catatagatccaagaaacccgctaagaacctcaagatgcctggcgtctactatgtggac | |
| | tacagactggaaagaatcaaggaggccaacaacgagacctacgtcgagcagcacgag | |
| | gtggcagtggccagatactgcgacctccctagcaaactgggCcacaagctaaatTA | |
| | A | |
| RR-EE Thy1.1 M1 VH-VL | METDTLLLWVLLLWVPGSTGLEIRAAFLRQRNTALRTE | 92 |
| CD24 ST CD28EC CD28 | VAELEQEVQRLENEVSQYETRYGPLGGGKGGGGSGGG | |
| 1XX 3xHA NFKBIAm | GSGGGGSGGGSLEIEAAFLERENTALETRVAELRQRVQ | |
| | RLRNRVSQYRTRYGPLGGGKEFTQKVTSLTACLVNQN | |
| | LRLDCRHENNTKDNSIQHEFSLTREKRKHVLSGTLGIPE | |
| | HTYRSRVTLSNQPYIKVLTLANFTTKDEGDYFCELRVS | |
| | GANPMSSNKSISVYRDKLVKCGGISLLVQNTSWMLLLL | |
| | LSLSLLQALDFISLGSGATNFSLLKQAGDVEENPGPGSL | |
| | TMASPLTRFLSLNLLLLGESIILGSGEAEVKLQESGPGLV | |
| | KPSLTLSLTCTVSGFSLNNYAVIWVRQPPGKGLEWMGV | |
| | IWSDGKTNYNSALKSRLSISRDTSKSQVFLKMNNLQTE | |
| | DTAMYFCARSSYYSSWFDSWGQGVMVTVSSGGGGSG | |
| | GGGSGGGGSDIQLTQSPSFLSASVGDRVTLSCKASQNIN | |
| | KYLAWYQQKLGEAPKLLIYNANSLQTGIPSRFSGSGSG | |
| | TDFTLTISSLQPEDVATYFCLQHNSWWTLGGGTKLELK | |
| | RSAWSHPQFEKGGGSGGGSGGSAWSHPQFEKGGGGSG | |
| | GGSEARLIEFMYPPPYLDNERSNGTIIHIKEKHLCHTQSS | |
| | PKLFWALVVVAGVLFCYGLLVTVALCVIWTNSRRNRL | |
| | LQSDYMNMTPRRPGLTRKPYQPYAPARDFAAYRPRAK | |
| | FSRSAETAANLQDPNQLYNELNLGRREEYDVLEKKRA | |
| | RDPEMGGKQQRRRNPQEGVFNALQKDKMAEAFSEIGT | |
| | KGERRRGKGHDGLFQGLSTATKDTFDALHMQTLAPRG | |
| | SGQCTNYALLKLAGDVESNPGPLEYPYDVPDYAGYPY | |
| | DVPDYAGYPYDVPDYAGSGMFQPAGHGQDWAMEGPR | |
| | DGLKKERLVDDRHDAGLDAMKDEEYEQMVKELREIRL | |
| | QPQEAPLAAEPWKQQLTEDGDSFLHLAIIHEEKPLTME | |
| | VIGQVKGDLAFLNFQNNLQQTPLHLAVITNQPGIAEALL | |
| | KAGCDPELRDFRGNTPLHLACEQGCLASVAVLTQTCTP | |
| | QHLHSVLQATNYNGHTCLHLASIHGYLAIVEHLVTLGA | |
| | DVNAQEPCNGRTALHLAVDLQNPDLVSLLLKCGADVN | |
| | RVTYQGYSPYQLTWGRPSTRIQQQLGQLTLENLQMLPE | |
| | AEDEEAYDAEAEFAEDELPDDDCVFGGQRLTL | |
| R2 3N PD-1EC CD28 | METDTLLLWVLLLWVPGSTGCPYSNPSLCSGGGGSSGG | 93 |
| delta P2A CD20 CD28EC | GGSDCPYSNPSLCGGGGSGGGSLEIEAAFLRQENTALRT | |
| CD28z | RVAELEQEVQRLENEVSQYETRYGPLGGGKGGGGSGG | |
| | GGSGGGGSGGGSLEIEAAFLERENTALETRVAELRQRV | |
| | QRLRNRVSQYRTRYGPLGGGKGGGGSGGGSTERILETS | |
| | TRYPSPSPKPEGRFQGMFWALVVVAGVLFCYGLLVTV | |
| | ALCVIWTRAKFSRSAETAANLQDTFDALHMQTLAPRGS | |
| | GATNFSLLKQAGDVEENPGPGSLTMASPLTRFLSLNLLL | |
| | LGESIILGSGEAQIVMSQSPAILSASPGEKVTMTCRARSS | |
| | VSYIHWYQQKPGSSPKPWIYATSNLASGVPGRFSGSGS | |
| | GTSYSLTITRVEAEDAATYYCQQWSSKPPTFGGGTKLEI | |
| | KRGGGGSGGGGSGGGGSQVQLQQPGAELVRPGTSVKL | |
| | SCKASGYTFTSYWMHWIKQRPGQGLEWIGVIDPSDNY | |
| | TKYNQKFKGKATLTVDTSSSTAYMQLSSLTSEDSAVYF | |
| | CAREGYYGSSPWFAYWGQGTLVTVSSSAWSHPQFEKG | |
| | GGSGGGSGGSAWSHPQFEKGGGGSGGGSEARLIEFMY | |
| | PPPYLDNERSNGTIIHIKEKHLCHTQSSPKLFWALVVVA | |
| | GVLFCYGLLVTVALCVIWTNSRRNRLLQSDYMNMTPR | |
| | RPGLTRKPYQPYAPARDFAAYRPRAKFSRSAETAANLQ | |
| | DPNQLYNELNLGRREEYDVLEKKRARDPEMGGKQQRR | |
| | RNPQEGVYNALQKDKMAEAYSEIGTKGERRRGKGHD | |
| | GLYQGLSTATKDTYDALHMQTLAPRGSGQCTNYALLK | |
| | LAGDVESNPGP | |

### 3. Enhancing CAR Expressing Immune Cell Function with Inhibitory Peptides

The presently disclosed subject matter provides systems, engineered immune cells, and methods using the same wherein the systems, cells, and methods comprise nucleic acid constructs comprising a nucleic acid sequence encoding an inhibitory peptide and a carrier protein (CP). In certain embodiments, the nucleic acid construct comprises a nucleic acid sequence encoding an inhibitory peptide:CP fusion. In certain embodiments, the inhibitory pepetide:CP fusion is oriented with the inhibitory peptide first, followed by the CP, while in certain embodiments the CP is first, followed by the inhibitory peptide.

### 3.1 NFAT Signaling Inhibitory Peptides

### 3.1.1 PXIXIT Calcineurin Binding Sequences

In certain embodiments, the inhibitory peptide inhibits the calcineurin signaling pathway. In certain embodiments, the inhibitory peptide blocks calcineurin dependent dephosphorylation of nuclear factor of activated T cells (NFAT) whereby, NFAT activation in inhibited. In certain embodiments, the NFAT inhibitor comprises a PXIXIT calcineurin binding sequence. In certain embodiments, the NFAT inhibitor comprises a PXIXIT calcineurin binding sequence in which X is any amino acid. Non-limiting exemplary NFAT signaling inhibitors comprise the amino acid sequence set forth in any one of SEQ ID NOs: 24-29.

### 3.1.2 Autoinhibitory Domain Sequence of Calcineurin

In certain embodiments, the inhibitory peptide comprises the autoinhibitory domain sequence in calcineurin. In certain embodiments, these peptides bind and block the ability of calcineurin to activate NFAT and can therefore be used to inhibit calcineurin dependent NFAT signaling. An exemplary autoinhibitory domain-based calcineurin/NFAT signaling inhibitor comprises the amino acid sequence set forth in SEQ ID NO: 30.

### 3.2 NFkB Inhibitors

In certain embodiments, the inhibitory peptide is a NFkB inhibitor. In a non-limiting example, the NFkB inhibitor is NFkB inhibitor alpha (NFKBIA). In certain embodiments, the inhibitory peptide is a degradation-resistant mutant of a NFkB inhibitor. In certain embodiments, the degradation-resistant mutant of a NFkB inhibitor is a mutated variant of NFkBIA, for e.g., NFkBIAm having a sequence set forth in SEQ ID No: 32.

### 4. Carrier Proteins

Carrier proteins, as used in connection with the presently disclosure subject matter, can be any protein to which an inhibitory peptide of the presently disclosed subject matter is operably linked, e.g., fused in the context of an inhibitor peptide:CP fusion protein. In certain embodiments, the fusion of a CP to an inhibitory peptide enables intracellular expression of an inhibitory peptide-carrier fusion protein that retains the inhibitory properties of the “free” peptide when not fused to the CP.

In certain embodiments, and not by way of any limitation, the carrier protein is glutathione-S-transferase (GSTA1), enhanced green fluorescence protein (EGFP), or blue fluorescent (BFP), or variants thereof.

In certain embodiments, the nucleic acid sequence for the CP encodes a regulatable gene element. In certain of the foregoing systems, the regulatable gene element encodes a regulator motif that regulates expression of the inhibitory peptide. In certain embodiments, the regulator motif comprises a drug-destabilized domain, where stable expression of the inhibitory peptide-CP fusion protein requires presence of a stabilizing drug. Non-limiting examples of drug-destabilized domain include a dihydrofolate reductase destabilization domain (DHFR-DD) and a FK506 binding protein destabilization domain (FKBP-DD). A non-limiting example of a drug that stabilizes DHFR-DD is trimethoprim (TMP). A non-limiting example of a drug that stabilizes FKBP-DD is Shield-1 (Shld1).

In certain embodiments, the drug-destabilized domain is DHFR-DD having an amino acid sequence set forth in SEQ ID NO: 43. In certain embodiments, the inhibitory peptide is NFKBIAm and the drug-destabilized domain is DHFR-DD, the fusion protein having an amino acid sequence set forth in SEQ ID NO: 33

### 4.1 Carrier Proteins Comprising Leucine Zipper Motifs

In certain embodiments, the CP is a membrane bound polypeptide, wherein the nucleic acid sequence for the CP encodes an extracellular domain comprising a first leucine zipper sequence (capture zipper), a transmembrane domain, and an intracellular domain, the system further comprising a second nucleic acid construct comprising a nucleic acid sequence encoding a soluble polypeptide attached to a second leucine zipper sequence (secreted zipper) that heterodimerizes with the first leucine zipper motif.

4.1.1 Membrane-Bound Polypeptide In certain embodiments, the membrane-bound polypeptide comprises a transmembrane domain and an extracellular domain. In certain embodiments, the membrane-bound polypeptide further comprises a hinge/spacer domain and/or an intracellular domain.

### 4.1.2 Extracellular Domain

In certain embodiments, the extracellular domain comprises a dimerization sequence that is capable of dimerizing with one or more dimerization sequences comprised in the membrane-bound polypeptide. In certain embodiments, the dimerization sequence is capable of dimerizing with one or more dimerization sequences comprised in a soluble polypeptide disclosed herein. In certain embodiments, the extracellular domain of the membrane-bound polypeptide comprises a first dimerization sequence and a second dimerization sequence that is capable of dimerizing with the first dimerization sequence at a cell surface. In certain embodiments, the extracellular domain comprises a dimerization sequence comprising a first leucine zipper sequence (capture zipper) that is capable of heterodimerizing with one or more dimerization sequences comprised in the soluble polypeptide disclosed herein. In certain embodiments, the extracellular domain comprises a dimerization sequence comprising a first leucine zipper sequence (capture zipper) that is capable of heterodimerizing with a second leucine zipper sequence (secreted zipper) comprised in the soluble polypeptide disclosed herein.

In certain embodiments, the leucine zipper sequence comprises a dimerization sequence of the Basic-region leucine zipper (bZIP) class of eukaryotic transcription factors. In certain embodiments, the leucine zipper sequence comprises a specific alpha helix monomer that can dimerize with anther alpha helix monomer. In certain embodiments, the leucine zipper sequence comprises an EE domain that comprises one or more acidic amino acids, e.g., glutamic acid (E). In certain embodiments, the leucine zipper sequence comprises an RR domain that comprises one or more basic amino acids, e.g., arginine (R). In certain embodiments, the second leucine zipper sequence comprises an RR domain and the first leucine zipper sequence comprises an EE domain.

In certain embodiments, the RR domain comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 35 or a fragment thereof. In certain embodiments, the RR domain comprises a modification of SEQ ID NO: 35 or a fragment thereof. In certain embodiments, the modification comprises up to one, up to two, or up to three amino acid substitutions.

In certain embodiments, the RR domain comprises a modification of SEQ ID NO: 35, wherein the modification consists of or has one amino acid substitution.

In certain embodiments, the RR domain comprises a modification of SEQ ID NO: 35, wherein the modification consists of or has three amino acid substitutions.

In certain embodiment, the modification is positioned in the “g” residues of the RR domain of the leucine zipper. In certain embodiment, the modification reduces heterodimerization affinity between the membrane-bound polypeptide and a linked soluble polypeptide.

In certain embodiments, the EE domain comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 34 or a fragment thereof. In certain embodiments, the EE domain comprises a modification of SEQ ID NO: 34 or a fragment thereof. In certain embodiments, the modification comprises up to one, up to two, or up to three amino acid substitutions.

In certain embodiments, the extracellular domain further comprises a linker between the first dimerization sequence and the second dimerization sequence. In certain embodiments, the linker comprises the amino acid sequence set forth in any one of SEQ ID NOs: 1-23.

In certain embodiments, a dimerization sequence comprises an orthogonal zipper. Orthogonal zippers are coiled coil domains that form heterodimers with their specific partner only and not with other zipper domains. In certain embodiments, orthogonality refers to sets of molecules (e.g., leucine zippers) that are non-cross-reactive, i.e., “orthogonal”, to other sets of molecules. For example, A+B=AB and C+D=CD, but neither A nor B bind to C or D, and vice versa.

In certain embodiments, the first and second leucine zipper sequences of the membrane-bound polypeptide are a pair of orthogonal zippers i.e., the first and the second leucine zipper sequences are the specific partners for each other to form heterodimers. Orthogonal zippers include, but are not limited to, RR/EE zippers, Fos/Jun zippers and Fos/synZip zippers. Fos/Jun zippers are previously disclosed in Ransone et al., Genes Dev. 1989 June; 3(6):770-81; Kohler et al., Biochemistry. (2001 January); 9; 40(1):130-42, which are incorporated by reference herein. Fos/synZip zippers are previously disclosed in Grigoryan et al., Nature. (2009); 458, 859-864; Reinke et al., J Am Chem Soc. (2010); 132, 6025-6031, which are incorporated by reference herein.

In certain embodiments, the orthogonal zippers comprise an amino acid sequence that is at least about 80%, at least about 85%, at least about 90%, at least about 95%, at least about 96%, at least about 97%, at least about 98%, at least about 99% or at least about 100% homologous or identical to RR/EE zippers, Fos/Jun zippers or Fos/synZip zippers, or a fragment thereof, and/or may optionally comprise up to one or up to two or up to three conservative amino acid substitutions.

In certain embodiments, the extracellular domain of the membrane-bound polypeptide further comprises a spacer/hinge domain between a dimerization sequence and a transmembrane domain.

In certain embodiments, the spacer/hinge domain is flexible enough to allow the dimerization sequence to orient in different directions to facilitate antigen recognition after dimerizing with the soluble polypeptide disclosed herein. The spacer region can be the hinge region from IgG1, or the CH₂CH₃ region of immunoglobulin and fragments of CD3, a fragment of a CD28 polypeptide, a fragment of a CD8 polypeptide, a variation of any of the foregoing that is at least about 80%, at least about 85%, at least about 90%, at least about 95%, at least about 96%, at least about 97%, at least about 98%, at least about 99% or at least about 100% identical thereto, or a synthetic spacer sequence. Non-limiting hinge domain amino acid sequences are set forth in any one of SEQ ID NOs: 52, 54 or 62;

In certain embodiments, the spacer/hinge domain comprises an epitope recognized by an antibody. In certain embodiments, binding of the antibody to the epitope mediates a deletion of a cell comprising the membrane-bound polypeptide. In certain embodiments, the spacer/hinge domain comprises a Thy1.1 molecule, a truncated EGFR molecule (EGFRt), CD22 immunoglobulin-like domain epitope, an IgG/Fc domain (can be a Fc from any IgG), CD2, CD20 cyclic mimotope, CD30, CD52, or HER2.

In certain embodiments, the Thy1.1 molecule comprises or has the amino acid sequence set forth in SEQ ID NO: 66.

In certain embodiments, the membrane-bound polypeptide further comprises a blocking spacer, wherein the blocking spacer is capable of preventing dimerization of the membrane-bound polypeptide with a soluble polypeptide when the membrane-bound polypeptide and the soluble polypeptide are not expressed from the same cell. In certain embodiments, the blocking spacer comprises a minimum spacer of no more than about 20 to about 30 amino acid residues. In certain embodiments, the blocking spacer comprises no more than about 25 amino acid residues. In certain embodiments, the blocking spacer comprises about 1, about 2, about 3, about 4, about 5, about 6, about 7, about 8, about 9 about 10, about 11, about 12, about 13, about 14, about 15, about 16, about 17, about 18, about 19 or about 20 amino acid residues. In certain embodiments, the blocking spacer comprises between about 5 amino acid residues and about 25 amino acid residues, between about 5 amino acid residues and about 20 amino acid residues, between about 10 amino acid residues and about 25 amino acid residues or between about 10 amino acid residues and about 20 amino acid residues.

In certain embodiments, the blocking spacer has a length of no more than about 25 amino acids. In certain embodiments, the blocking spacer has a length of between about 5 amino acids and about 25 amino acids. In certain embodiments, the blocking spacer is a truncated CD28 spacer or an IgG1 hinge.

In certain non-limiting embodiments, the extracellular domain of the membrane-bound polypeptide comprises at least one co-stimulatory ligand or a fragment thereof.

### 4.1.3. Transmembrane Domain

Different transmembrane domains can result in different receptor stabilities. In accordance with the presently disclosed subject matter, the transmembrane domain can comprise a CD8 polypeptide (e.g., the transmembrane domain of CD8 or a fragment thereof), a CD28 polypeptide (e.g., the transmembrane domain of CD28 or a fragment thereof), a CD3ζ polypeptide (e.g., the transmembrane domain of CD3ζ or a fragment thereof), a CD4 polypeptide (e.g., the transmembrane domain of CD4 or a fragment thereof), a 4-1BB polypeptide (e.g., the transmembrane domain of 4-1BB or a fragment thereof), an OX40 polypeptide (e.g., the transmembrane domain of OX40 or a fragment thereof), an ICOS polypeptide (e.g., the transmembrane domain of ICOS or a fragment thereof), a CD2 polypeptide (e.g., the transmembrane domain of CD2 or a fragment thereof), a synthetic peptide (not based on a protein associated with the immune response), or a combination thereof.

In certain embodiments, the transmembrane domain of the membrane-bound polypeptide comprises a CD8 polypeptide (e.g., the transmembrane domain of CD8 or a fragment thereof). In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001139345.1 (SEQ ID NO: 45) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 45, which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, 183 to 203, or 200 to 235 of SEQ ID NO: 45. In certain embodiments, the transmembrane domain of the membrane-bound polypeptide comprises a CD8 polypeptide comprising or having an amino acid sequence of amino acids 183 to 203 of SEQ ID NO: 45.

In certain embodiments, the CD8 polypeptide comprises or has the amino acid sequence set forth in SEQ ID NO: 63.

In certain embodiments, the transmembrane domain of the membrane-bound polypeptide comprises a CD28 polypeptide (e.g., the transmembrane domain of CD28 or a fragment thereof). The CD28 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P10747 or NP_006130, or a fragment thereof, and/or may optionally comprise up to one or up to two or up to three conservative amino acid substitutions. In certain embodiments, the CD28 polypeptide comprises or has an amino acid sequence that is a consecutive fragment of which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, 153 to 179, or 200 to 220 of the sequence having NCBI Reference No: P10747 or NP_006130. In certain embodiments, the transmembrane domain of a presently disclosed membrane-bound polypeptide comprises a CD28 polypeptide comprising or having an amino acid sequence of amino acids 153 to 179 of the sequence having NCBI Reference No: P10747 or NP_006130. In certain embodiments, the transmembrane domain of a membrane-bound polypeptide comprises a CD28 polypeptide comprising or having the amino acid sequence set forth in SEQ ID NO: 53.

### 4.1.4 Intracellular (Cytoplasmic) Domain

In certain embodiments, the membrane-bound polypeptide further comprises an intracellular domain. In certain non-limiting embodiments, the intracellular domain comprises a cytoplasmic stalk to which can be attached the inhibitory peptide-carrier protein fusion.

In certain non-limiting embodiments, the intracellular domain provides an activation signal to a cell (e.g., a cell of the lymphoid lineage, e.g., a T cell). In certain embodiments, the intracellular domain of the membrane-bound polypeptides comprises an immune activating molecule. In certain embodiments, the immune activating molecule is a CD3ζ polypeptide. In certain embodiments, the immune activating molecule is a CD3ζ polypeptide having an amino acid sequence set forth in any one of SEQ ID NOs: 57 or 58.

In certain embodiments, the intracellular domain comprises a member of the immunoglobulin superfamily. In a non-limiting embodiment, the intracellular domain comprises an intracellular domain of CD28.

In certain embodiments, the intracellular domain of the membrane-bound polypeptide comprises a murine CD3ζ polypeptide.

In certain embodiments, the intracellular domain of the membrane-bound polypeptide comprises a human CD3ζ polypeptide.

In certain non-limiting embodiments, the intracellular domain of the membrane-bound polypeptide provides an activation signal and a stimulation signal to a cell. In certain embodiments, the intracellular of the membrane-bound polypeptide domain comprises at least one costimulatory molecule or a fragment thereof.

In certain embodiments, the at least one co-stimulatory signaling region comprises a CD28 polypeptide (e.g., the intracellular domain of CD28 or a fragment thereof), a 4-1BB polypeptide (e.g., the intracellular domain of 4-1BB or a fragment thereof), an OX40 polypeptide (e.g., the intracellular domain of OX40 or a fragment thereof), an ICOS polypeptide (e.g., the intracellular domain of ICOS or a fragment thereof), a DAP-10 polypeptide (e.g., the intracellular domain of DAP-10 or a fragment thereof), or a combination thereof. In certain embodiments, the at least one co-stimulatory signaling region comprises a CD28 polypeptide. In certain embodiments, the at least one co-stimulatory signaling region comprises an intracellular domain of CD28 or a fragment thereof.

In certain embodiments, the costimulatory molecule is a CD28 polypeptide (e.g., the intracellular domain of CD28 or a fragment thereof). The CD28 polypeptide can comprise or have an amino acid sequence that is at least about 80%, at least about 85%, at least about 90%, at least about 95%, at least about 96%, at least about 97%, at least about 98%, at least about 99% or at least 100% homologous or identical to the sequence having a NCBI Reference No: P10747 or NP_006130 or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 92 which is at least 20, or at least 30, or at least 40, or at least 50, and up to 220 amino acids in length.

In certain embodiments, the CD28 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_031668.3 or a fragment thereof, and/or may optionally comprise up to one or up to two or up to three conservative amino acid substitutions.

In certain embodiments, the costimulatory molecule is a mouse CD28 polypeptide. In certain embodiments, the costimulatory molecule is a human CD28 polypeptide.

In certain embodiments, the intracellular domain of the membrane-bound polypeptide comprises two costimulatory molecules, e.g., CD28 and 4-1BB or CD28 and OX40.

In certain embodiments, the at least one co-stimulatory signaling region comprises a 4-1BB polypeptide. In certain embodiments, the at least one co-stimulatory signaling region comprises an intracellular domain of 4-1BB or a fragment thereof.

In certain embodiments, the costimulatory molecule is a 4-1BB polypeptide (e.g., the intracellular domain of 4-1BB or a fragment thereof). 4-1BB can act as a tumor necrosis factor (TNF) ligand and have stimulatory activity. The 4-1BB polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P41273 or NP_001552 or a fragment thereof, and/or may optionally comprise up to one or up to two or up to three conservative amino acid substitutions.

In accordance with the presently disclosed subject matter, a “4-1BB nucleic acid molecule” refers to a polynucleotide encoding a 4-1BB polypeptide.

### 4.1.5 Soluble Polypeptide

In certain embodiments, the soluble polypeptide comprises a dimerization sequence that is capable of dimerizing with a dimerization sequence comprised in a membrane-bound polypeptide disclosed herein. In certain embodiments, the membrane-bound polypeptide is a membrane-bound polypeptide disclosed herein. In certain embodiments, the dimerization sequence comprises a leucin zipper domain. The dimerization sequence can be any of the dimerization sequences disclosed herein.

In certain embodiments, the soluble polypeptide comprises a dimerization sequence and an antigen-binding domain that is capable of binding to an antigen.

In certain embodiments, the soluble polypeptide comprises a dimerization sequence and a cytokine or a chemokine. In certain embodiments, the soluble polypeptide further comprises a tag.

In certain embodiments, the leucine zipper sequence of the membrane-bound polypeptide and the leucine zipper sequence of the soluble polypeptide are a pair of orthogonal zippers, i.e., they are the specific partners for each other to form heterodimers.

### 4.1.6 Tags

In certain embodiments, the soluble polypeptide further comprises a tag. In certain embodiments, the tag comprises an epitope tag, which comprises an epitope recognized by an antibody. In certain embodiments, the epitope tag is selected from the group consisting of a Myc-tag, a HA-tag, a Flag-tag, a V5-tag, a T7 tag, and combinations thereof. In certain embodiments, the tag comprises an affinity tag that binds to a substrate. In certain embodiments, the affinity tag is selected from the group consisting of a His-tag, a Strep-tag, an E-tag, a streptavidin binding protein tag (SBP-tag), and combinations thereof.

Furthermore, the soluble polypeptide can further comprise a mimotope recognized by a second antibody. Binding of the second antibody to the mimotope can mediates depletion of a cell comprising the membrane-bound polypeptide. In certain embodiments, the mimotope is a CD20 mimotope recognized by an anti-CD20 antibody. In certain embodiments, the anti-CD20 antibody is Rituxumab. In certain embodiments, the CD20 mimotope is a circular CD20 mimotope.

In certain embodiments, the CD20 mimotope comprises or has the amino acid sequence set forth in SEQ ID NO: 51.

### 5. Reporters

In certain embodiments, the nucleic acid constructs of the present disclosure can further comprise a nucleotide sequence encoding a reporter protein, wherein expression of the reporter protein enables identification of a cell expressing an inhibitory peptide. Non-limiting examples of such reporters include, EGFP, GFP, BFP and luciferase.

### 6. Promoters for Driving NFAT and/or NFkB Inhibitor Expression

The presently disclosed subject matter provides systems, engineered immune cells, and methods using the same wherein the systems, cells, and methods comprise nucleic acid constructs comprising a nucleic acid sequence encoding an inhibitory peptide and a carrier protein, as well as a promoter element that regulates transcription of the inhibitory peptide-carrier protein fusion construct. The use of promoter elements in this manner enables a negative feedback loop that inhibits excessive NFAT signaling in the transduced cells when encountering targets with high antigen expression for extended durations, while avoiding NFAT inhibition against weaker targets that induce NFAT to a lesser extent. Non-limiting examples of such promoter elements include a NFAT and/or NFkB responsive promoter elements. In certain embodiments, the promoter element is a NFAT responsive promoter element. In certain embodiments, the NFAT responsive promoter element has a sequence set forth in SEQ ID No: 48. In certain embodiments, the promoter element is an NFkB responsive promoter element. In certain embodiments, the NFkB responsive promoter element has a sequence set forth in SEQ ID No: 49.

### 7. Antigens

In certain embodiments, the antigen binding domain of the soluble polypeptides and/or the CARs of the presently disclosed subject matter binds to a tumor antigen. Any tumor antigen can be used in the tumor-related embodiments described herein. The antigen can be expressed as a peptide or as an intact protein or fragment thereof. The intact protein or a fragment thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LlCAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, ZDHHC11, CD2, CD3, CD4, CD5, CD19, VpreB, CD40, CD79a, CD70b, CLL-1/CLEC12A, IL-3R complex, TIM-3, TACI, SLAMF7, CD244, E-cadherin, B7-H4, carbonic anhydrase IX (CAlX), carcinoembryonic antigen (CEA), an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), k-light chain, kinase insert domain receptor (KDR), L1 cell adhesion molecule (L1CAM), 1 (MAGE-A1), Proteinase3 (PR1), cancer-testis antigen NY-ESO-1, tumor-associated glycoprotein 72 (TAG-72), and Wilms tumor protein (WT-1), PRAME and ERBB variants thereof, or combinations thereof.

In certain embodiments, the antigen binding domain of the soluble polypeptide binds to a human CD19 polypeptide. In certain embodiments, the antigen binding domain of the soluble polypeptide binds to the extracellular domain of a human CD19 protein.

In certain embodiments, the antigen binding domain of the soluble polypeptide binds to an immune checkpoint molecule. Non-limiting example of immune checkpoint molecules include PD-L1, CD200, B7-H3, B7-H4, HVEM, Galectin9, PD-1, CTLA-4, CD200R1, TIM-3, Lag-3 and TIGIT.

In certain embodiments, the antigen binding domain of the soluble polypeptide binds to an activating receptor, wherein the binding of the antigen binding domain to the activating receptor is capable of activating an antigen presenting cell (APC). Non-limiting example of immune checkpoint molecules include CD40, Toll Like Receptors (TLRs), FLT3, RANK, and GM-CSF receptor.

In certain embodiments, the antigen binding domain of the soluble polypeptide binds to a biomarker of a hematopoietic lineage cell. Non-limiting example of immune checkpoint molecules include CD3, CD16, CD33, c-Kit, CD161, CD19, CD20, VpPreB, luteinizing hormone receptor (LHCGR), CD123, IL-3R complex, CLEC12A/CLL-1.

In certain embodiments, the antigen binding domain of the soluble polypeptide binds to a pathogen antigen, e.g., for use in treating and/or preventing a pathogen infection or other infectious disease, for example, in an immunocompromised subject. Non-limiting examples of pathogens include a virus, bacteria, fungi, parasite and protozoa capable of causing disease.

Non-limiting examples of viruses include, Retroviridae (e.g., human immunodeficiency viruses, such as HIV-1 (also referred to as HDTV-III, LAVE or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronoviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Naira viruses); Arena viridae (hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), the agents of non-A, non-B hepatitis (class 1=internally transmitted; class 2=parenterally transmitted (e.g., Hepatitis C); Norwalk and related viruses, and astroviruses).

Non-limiting examples of bacteria and/or fungi include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringerns, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Aspergillus species and Actinomyces israelli.

### 8. Exemplary Constructs

In certain embodiments, the present disclosure is directed to a VIVIT 3xHA DHFR-DD N type hCD8 construct comprising an inhibitor polypeptide-carrier fusion protein. In certain embodiments, the construct comprises SEQ ID NO: 71.

In certain embodiments, the present disclosure is directed to a Vmut3 3xHA DHFR-DD N type hCD8 construct comprising an inhibitor polypeptide-carrier fusion protein. In certain embodiments, the construct comprises SEQ ID NO: 72.

In certain embodiments, the present disclosure is directed to a VIVIT GSTA1 3xHA hCD8 construct comprising an inhibitor polypeptide-carrier fusion protein. In certain embodiments, the construct comprises SEQ ID NO: 73.

In certain embodiments, the present disclosure is directed to a Vmut3 GSTA1 3xHA hCD8 construct comprising an inhibitor polypeptide-carrier fusion protein. In certain embodiments, the construct comprises SEQ ID NO: 74.

In certain embodiments, the present disclosure is directed to a VIVIT GFP-Vmut1 construct comprising an inhibitor polypeptide-carrier fusion protein. In certain embodiments, the construct comprises SEQ ID NO: 75.

In certain embodiments, the present disclosure is directed to a VIVIT GFP-Vmut2 construct comprising an inhibitor polypeptide-carrier fusion protein. In certain embodiments, the construct comprises SEQ ID NO: 76.

In certain embodiments, the present disclosure is directed to a VIVIT GFP-Vmut3 construct comprising an inhibitor polypeptide-carrier fusion protein. In certain embodiments, the construct comprises SEQ ID NO: 77.

In certain embodiments, the present disclosure is directed to a VIVIT GFP-Vmut5 construct comprising an inhibitor polypeptide-carrier fusion protein. In certain embodiments, the construct comprises SEQ ID NO: 78.

In certain embodiments, the present disclosure is directed to a VIVIT GFP NFAT SFFV BFP construct comprising an inhibitor polypeptide-carrier fusion protein. In certain embodiments, the construct comprises SEQ ID NO: 79.

In certain embodiments, the present disclosure is directed to a VIVIT GFP NFkB SFFV BFP construct comprising an inhibitor polypeptide-carrier fusion protein. In certain embodiments, the construct comprises SEQ ID NO: 80.

In certain embodiments, the present disclosure is directed to a R2 3N PD-1EC CD28 delta Vmut3 P2A CD20 CD28EC CD28z construct comprising an inhibitor polypeptide-carrier fusion protein. In certain embodiments, the construct comprises SEQ ID NO: 81.

In certain embodiments, the present disclosure is directed to a R2 3N PD-1EC CD28 delta Vmut3 3×P2A CD20 CD28EC CD28z construct comprising an inhibitor polypeptide-carrier fusion protein. In certain embodiments, the construct comprises SEQ ID NO: 82.

In certain embodiments, the present disclosure is directed to a R2 3N PD-1EC CD28 delta DAMP P2A CD20 CD28EC CD28z construct comprising an inhibitor polypeptide-carrier fusion protein. In certain embodiments, the construct comprises SEQ ID NO: 83.

In certain embodiments, the present disclosure is directed to a EGFP-DAMP. In certain embodiments, the construct comprises SEQ ID NO: 84.

In certain embodiments, the present disclosure is directed to a Q2 RR12EE345L 2A iC9 CD19 CD8EC CD28z construct comprising an inhibitor polypeptide-carrier fusion protein. In certain embodiments, the construct comprises SEQ ID NO: 85.

In certain embodiments, the present disclosure is directed to a FLAG-RR12EE345L P2A iC9 F2A CD19 CD8H CD8TM BBz construct comprising an inhibitor polypeptide-carrier fusion protein. In certain embodiments, the construct comprises SEQ ID NO: 86.

In certain embodiments, the present disclosure is directed to a RR12EE345L linker EE12RR345L Thy1.1 P2A CD20 CD28H CD28TM BBz E2A construct comprising an inhibitor polypeptide-carrier fusion protein. In certain embodiments, the construct comprises SEQ ID NO: 87.

In certain embodiments, the present disclosure is directed to a FLAG-RR12EE345L P2A iC9 F2A 3xHA NFKBIAm E2A CD19 CD8H CD8TM BBz construct comprising an inhibitor polypeptide-carrier fusion protein. In certain embodiments, the construct comprises SEQ ID NO: 88.

In certain embodiments, the present disclosure is directed to a RR12EE345L linker EE12RR345L Thy1.1 P2A CD20 CD28H CD28TM BBz E2A 3xHA NFKBIAm construct comprising an inhibitor polypeptide-carrier fusion protein. In certain embodiments, the construct comprises SEQ ID NO: 89.

In certain embodiments, the present disclosure is directed to a RR12EE345L linker EE12RR345L Thy1.1 CD20 CD28EC BBz 3xHA DHFR-DD NFKBIAm construct comprising an inhibitor polypeptide-carrier fusion protein. In certain embodiments, the construct comprises SEQ ID NO: 90.

In certain embodiments, the present disclosure is directed to a NFKBIAm NFAT SFFV BFP construct comprising an inhibitor polypeptide-carrier fusion protein. In certain embodiments, the construct comprises SEQ ID NO: 91.

In certain embodiments, the present disclosure is directed to a RR-EE Thy1.1 M1 V_{H}-VL CD24 ST CD28EC CD28 1XX 3xHA NFKBIAm construct comprising an inhibitor polypeptide-carrier fusion protein. In certain embodiments, the construct comprises SEQ ID NO: 92.

In certain embodiments, the present disclosure is directed to a R2 3N PD-1EC CD28 delta P2A CD20 CD28EC CD28z construct comprising an inhibitor polypeptide-carrier fusion protein. In certain embodiments, the construct comprises SEQ ID NO: 93.

### 9. Cells

In certain embodiments, the presently disclosed subject matter is directed to CAR expressing immune cells, e.g., CAR T cells, modified to inhibit NFAT and/or NFkB signaling. For example, the systems of the present disclosure enable the generation of T cells engineered to express a single CAR or multiple combinations of CARs and one or more inhibitors of NFAT and/or NFkB signaling. In certain embodiments, the presently disclosed subject matter relates to engineered cells comprising a leucine zipper-based system to facilitate the inhibition of NFAT and/or NFkB signaling, where the leucine zipper-based system enables single-step immunomagnetic sorting of cells transduced with two vectors with the goal of doubling the amount of genetic information delivered and promoting enhanced transgene expression provides engineered cells comprising a membrane-bound polypeptide, a soluble polypeptide and/or a system disclosed herein. In certain embodiments, the cells of the present disclosure can be transduced with the constructs encoding the polypeptides disclosed herein and/or the systems such that the cells co-express the polypeptides and/or the system. In certain embodiments, the cell is an immunoresponsive cell. The cell can be a cell of the lymphoid lineage or a myeloid lineage.

Cells of the lymphoid lineage can produce antibodies, regulate the cellular immune system, detect foreign agents in the blood, and detect cells foreign to the host, and the like. Non-limiting examples of cells of the lymphoid lineage include T cells, B cells, dendric cells, Natural Killer (NK) cells, cells from which lymphoid cells may be differentiated. In certain embodiments, the stem cell is a pluripotent stem cell. In certain embodiments, the pluripotent stem cell is an embryonic stem cell or an induced pluripotent stem cell.

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of any polypeptide or system disclosed herein. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a Natural killer cell. Natural killer (NK) cells can be lymphocytes that are part of cell-mediated immunity and act during the innate immune response. NK cells do not require prior activation in order to perform their cytotoxic effect on target cells.

In certain embodiments, the cells are human lymphocytes. In certain embodiments, the human lymphocytes comprise without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The cells (e.g., T cells) can be autologous, non-autologous (e.g., allogeneic), or derived in vitro from engineered progenitor or stem cells.

In certain embodiments, the cells of are cells of the myeloid lineage. In certain embodiments, the cells of the myeloid lineage comprise, without limitation, monocytes, macrophages, basophils, neutrophils, eosinophils, mast cell, erythrocyte, and thrombocytes.

The presently disclosed cells are capable of modulating the tumor microenvironment. Tumors have a microenvironment that suppresses the host immune response using any of a series of mechanisms by malignant cells to protect themselves from immune surveillance, recognition and elimination. Immune suppressive factors include but are not limited to infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred modified T cells (e.g., CAR T cells) upon encounter with targeted tumor cells.

In certain embodiments, the presently disclosed cells have increased cell persistence. In certain embodiments, the presently disclosed cells have decreased apoptosis and/or anergy.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. Monoclonal antibodies (mAbs) are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In certain embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be distinguished from dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

The presently disclosed subject matter provides an engineered immune cell comprising the system disclosed herein. In certain embodiments, the immune cell is a T cell. In certain embodiments, the immune cell is a CAR T cell. In certain embodiments, the CAR T cell expresses at least one CAR. In certain embodiments, the CAR is selective to one or more target cells of interest.

The presently disclosed subject matter also provides an engineered T cell comprising (i) at least one CAR comprising an antigen binding domain, a transmembrane domain, and an intracellular signaling domain; and (ii) the system disclosed herein.

In certain embodiments, the CAR is selective to one or more target cells of interest.

In certain embodiments, the CAR binds one or more antigens selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LlCAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

### 10. Nucleic Acid Compositions and Vectors

Genetic modification of a cell (e.g., a T cell) can be accomplished by transducing a substantially homogeneous cell composition with a recombinant DNA construct. In certain embodiments, a retroviral vector is employed for the introduction of the DNA construct into the cell. For example, a polynucleotide encoding any polypeptide or system disclosed herein can be cloned into a retroviral vector and expression can be driven from its endogenous promoter, from the retroviral long terminal repeat, or from a promoter specific for a target cell type of interest. In certain embodiments, the retroviral vector is a gammaretroviral vector. In certain embodiments, the retroviral vector is a lentiviral vector. Non-viral vectors may be used as well.

For initial genetic modification of a cell to include a polypeptide and/or a system disclosed herein, a retroviral vector is generally employed for transduction, however any other suitable viral vector or non-viral delivery system can be used. The polypeptides and/or the system can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits a high efficiency of infection, stable integration into the host cell genome, and durable expression of the recombinant gene product(s) (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALENs nucleases, CRISPR). Transient expression may be obtained by RNA electroporation. In certain embodiments, recombinant receptors can be introduced by a transposon-based vector. In certain embodiments, the transposon-based vector comprises a transposon (a.k.a. a transposable element). In certain embodiments, the transposon can be recognized by a transposase. In certain embodiments, the transposase is a Sleeping Beauty transposase.

The resulting cells can be grown under conditions similar to those for unmodified cells, whereby the modified cells can be expanded and used for a variety of purposes.

### 11. Polypeptides and Analogs

The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally occurring polypeptide disclosed herein (including, but not limited to, CD8, CD28, CD80, 4-1BBL, and CD3z). Analogs can differ from a naturally occurring polypeptide disclosed herein by amino acid sequence differences, by post-translational modifications, or by both. Analogs can exhibit at least about 80%, at least about 85%, at least about 90%, at least about 95%, at least about 96%, at least about 97%, at least about 98%, at least about 99% or at least about 100% homologous to all or part of a naturally occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2 d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein/peptide disclosed herein. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in a cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 12. Administration

Compositions comprising the presently disclosed cells can be provided systemically or directly to a subject for inducing and/or enhancing an immune response to an antigen and/or treating and/or preventing a neoplasia, pathogen infection, or infectious disease. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed cells and a pharmaceutically acceptable carrier. Administration can be autologous or heterologous. For example, cells can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 13. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10⁴ and about 10¹⁰, between about 10⁵ and about 10⁹, or between about 10⁶ and about 10⁸ of the presently disclosed cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein and, the time for sequential administrations can be ascertained without undue experimentation.

### 14. Methods

The presently disclosed subject matter provides methods for treating a disease comprising providing to a subject in need thereof a population of modified cells comprising the system disclosed herein; or a cell modified according to the method disclosed herein; or an enriched population of cells according to the method disclosed herein.

In certain embodiments, the subject is a human subject.

In certain embodiments, the disease is a cancer, an autoimmune disease, an inflammatory disease, or a graft versus-host disease. Non-limiting examples of cancer include cancer is leukemia, lymphoma, myeloma, ovarian cancer, breast cancer, bladder cancer, brain cancer, colon cancer, intestinal cancer, liver cancer, lung cancer, pancreatic cancer, prostate cancer, testicular cancer, anal cancer, skin cancer, stomach cancer, glioblastoma, throat cancer, melanoma, neuroblastoma, adenocarcinoma, glioma, or soft tissue sarcoma.

In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

In certain embodiments, the lymphoma is Hodgkin's lymphoma or non-Hodgkin's lymphoma.

In certain embodiments, the non-Hodgkin's lymphoma is B-cell non-Hodgkin's lymphoma or T-cell non-Hodgkin's lymphoma.

In certain embodiments, the cancer comprises cells expressing CD19 or CD20.

In certain embodiments, the cancer comprises cells expressing at least one antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LlCAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Polypeptidease3 (PR1), PSD2, PTPRJ, RDH16, receptor tyrosine-polypeptide kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycopolypeptide 72 (TAG-72), Tyrosinase, vascular endothelial growth factor R2 (VEGF-R2), VLA-4, Wilms tumor polypeptide (WT-1), WNT4, WT1, and ZDHHC11.

The methods comprise administering to a subject an effective amount of the cells disclosed herein or a pharmaceutical composition comprising such cells. The presently disclosed cells and compositions comprising thereof can be used for treating and/or preventing neoplasia in a subject. The presently disclosed cells and compositions comprising thereof can be used for prolonging the survival of a subject suffering from a neoplasm. The presently disclosed cells and compositions comprising thereof can also be used for treating and/or preventing a pathogen infection or other infectious disease in a subject, such as an immunocompromised human subject. Such methods comprise administering an amount effective the presently disclosed cells or a composition (e.g., a pharmaceutical composition) comprising such cells to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse, or slow the progression of the disease, or otherwise reduce the pathological consequences of the disease. The effective amount is generally determined by the physician on a case-by-case basis and is within the skill of one in the art. Several factors are typically considered when determining an appropriate dosage to achieve an effective amount. These factors include age, sex and weight of the subject, the condition being treated, the severity of the condition and the form and effective concentration of the cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal and directly to the thymus.

The treatment subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasia but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the cells described herein in treatment prophylactically to prevent the occurrence of neoplasia until it is suitable to perform preventive surgery.

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells or a composition (e.g., a pharmaceutical composition) comprising such cells to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

The presently disclosed subject matter further provides methods for increasing an immune activity of an immunoresponsive cell. In certain embodiments, the method comprises introducing to the immunoresponsive cell a system disclosed herein to the immunoresponsive cell.

The presently disclosed subject matter provides methods for activating an antigen presenting cell (APC) in a subject. In certain embodiments, the method comprises administering to the subject an effective amount of the cells or a composition (e.g., a pharmaceutical composition) comprising such cells.

The presently disclosed subject matter provides methods for conditioning a subject for bone marrow transplant. In certain embodiments, the method comprises administering to the subject an effective amount of the cells or a composition (e.g., a pharmaceutical composition) comprising such cells.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), and inducible Caspase 9 Suicide gene (iCasp-9). In certain embodiments, the cells include a truncated human epidermal growth factor receptor (EGFRt) polypeptide. The EGFRt polypeptide can enable T cell elimination by administering anti-EGFR monoclonal antibody (e.g., cetuximab). EGFRt can be covalently joined to the upstream of any polypeptide disclosed herein. The suicide gene can be included within the vector comprising nucleic acids encoding any polypeptide disclosed herein. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated T cells comprising any polypeptide or system disclosed herein. The incorporation of a suicide gene or EGFRt into the presently disclosed polypeptide or system gives an added level of safety with the ability to eliminate the majority of the engineered T cells within a very short time period. A presently disclosed cell (e.g., a T cell) incorporated with a suicide gene can be pre-emptively eliminated at a given timepoint post engineered T cell infusion or eradicated at the earliest signs of toxicity.

### 15. Examples

### 15.1 Materials and Methods

### 15.1.1 Vector and DNA Construct Design

Constructs were expressed from retroviruses or transposons. Table 1 shows the sequences for exemplary construct and construct elements described herein. Vector construct maps are shown in the figures. CAR and other receptor constructs used murine protein sequences except for Fas 4-1BB, which utilized the human 4-1BB costimulatory domain. DNA constructs were generated via standard molecular biology techniques including overlap extension PCR and restriction site-based cloning and ligated directly into pENTR1a no ccdB (Addgene 17398) or the MMLV-based retroviral vector LZRS-Rfa (Addgene 31601). SnapGene 6.2 (SnapGene) was used to design vectors. DNA templates were obtained as gBlocks or gene syntheses from Integrated DNA Technologies (IDT), or purchased from Origene, Addgene, and Sino Biological. pENTR1a-based constructs were transferred via Gateway cloning (Gateway LR Clonase II Enzyme mix, Invitrogen, 11791020) to LZRS-Rfa, piggybac transposons, or piggybac transposon ITR-flanked retroviral vectors containing attR recombination sites, designed in this study. These vectors were designed for stable high-level expression of retroviral vectors integrated into packaging lines using piggybac transposition. The vectors, named PB-MMLV-puro, PB-MPSV-puro, and PB-SIN-puro include the backbone and ITR and insulator domains of the piggybac transposon vector PB-EF1α-MCS-IRES-GFP (System Biosciences PB530A-2) and contain a 5′ compound SV40 enhancer with hybrid RSV-MMSV LTR (based on SERS11 design), leader sequence containing a modified MESV packaging signal (from MP71), attR sites flanking ccdB and chloramphenicol resistance genes, woodchuck hepatitis virus post-transcriptional regulatory element (WPRE), and followed by 3′ LTR regions from LZRS (MMLV), MP71 (MPSV), or inactivated MMLV LTR from pSIN (SIN). These vectors also contain an hPGK-promoter driving a Thy1.2-T2A-puroR selection cassette. PB-SFG5.3-BlastR contains the full 5′ LTR and packaging signal from SFG, attR sites flanking ccdB and chloramphenicol resistance genes, WPRE, 3′ SFG LTR, and hPGK-promoter driving a Thy1.2-T2A-BlastR selection cassette. The transposon PB-EF-1α-intron was generated by replacing the EF-1α core promoter in PB-EF1α-MCS-IRES-GFP with the full EF-1α intron-containing promoter. PB-EF-1α-intron-WPRE contains an added 3′ WPRE. gLuc-PD-1H-CD24-GPI-P2A-EGFP encodes a surface-expressed Gaussia luciferase with PD-1 hinge and membrane anchor based on mouse CD24 GPI transfer signal to localize BLI signal to gLuc-expressing cells and also encodes an EGFP reporter. CBR-P2A-hCD8-T2A-puroR is a construct containing click beetle red luciferase (CBR) along with a human CD8 surface reporter and encoding puromycin resistance. PB-SIN-puro BFP-SV40-NFAT-dEGFP, PB-SIN-puro BFP-SV40-AP-1-dEGFP, and PB-SIN-puro BFP-SV40-NFkB-dEGFP are transcription factor reporter vectors, and PB-EF-1α-intron hCD8-T2A-integrin-alpha-V and PB-EF-1α-intron mCD4-P2A-integrin-beta-3 encode components of human vitronectin receptor and hCD8 and mCD4 reporters, respectively.

### 15.1.2 Engineering a Leucine-Zipper Based Sorting System

To develop a platform to simplify purification of dual-vector-transduced cells by enabling single-step magnetic-bead cell sorting system, we utilized a heterodimerizing leucine zipper pair encoded by two vectors: (1) a secreted affinity-tagged zipper and (2), a membrane-bound capture-zipper designed to pair uniquely in double-transduced cells. This leucine zipper pair, termed RR12EE345L and EE12RR345L, was engineered for high-affinity heterodimerization via charge-based attraction of arginine (R) and glutamate (E) residues (Table 1).

### 15.1.3 Retrovirus Production

Retroviruses were produced in Phoenix-Eco or Phoenix-Eco alpha-V beta-3, designed to enhance adhesion of Phoenix-Eco to culture flasks. Phoenix-Eco or Phoenix-Eco alpha-V beta-3 were transfected with LZRS-vector constructs using Effectene® transfection reagent (Qiagen, 301425) as follows: complexes were generated using 2 μg vector DNA in 16 μL of enhancer, 20 μL of Effectene® and used to transfect Phoenix cells plated on a 10 cm dish at 2.5×10⁶ cells one day prior to transfection. On the day following transfection, cells were removed from dishes and assessed by flow cytometry for construct expression and selected in puromycin 2 μg/mL (Santa Cruz Biotech, sc-108071B). Phoenix-Eco or Phoenix-Eco alpha-V beta-3 were transfected with PB-vector constructs as above, with addition of 1 μg of the hyperactive piggyBac transposase vector pCMV-hyPBase⁷⁰ (Sanger Institute, UK) for stable integration. Transfected cells were selected one day after transfection with puromycin 2 μg/mL (Santa Cruz Biotech, sc-108071B) or blasticidin 10 μg/mL (Santa Cruz Biotechnology, sc-204655). Retroviral supernatant was collected from fully selected stable packaging lines grown from T175 flasks. Supernatants were filtered through 50 mL syringes fitted with Millex-HV Syringe Filters (Millipore, 0.45 μm, PVDF, SLHVR33RS) and polyethylene glycol solution concentrate was added (5× concentrate: PEG 8000 MW Promega, V3011, 40% weight/volume containing 2.4% NaCl weight/volume), and virus was precipitated over 1-2 days at 4 C. Precipitated virus was centrifuged at 3000×g for 15 minutes at 4 C and pellets were resuspended in 500 μL of T cell media and frozen at −80 C or used directly.

### 15.1.4 Retroviral Transduction

Tumor cell lines were transduced with retroviral supernatant containing polybrene 8 μg/mL (Sigma, H9268-10G) in 24-well plates by spinfection at 1500 RPM at 32 C for 1 hour. Cells were transferred to T25 flasks on the following day for expansion. For primary mouse T cell transduction, splenic T cells were enriched by negative selection of splenocytes with anti-CD19 microbeads to deplete B cells (Miltenyi, 130-121-301) and were stimulated on plate-bound anti-CD3/CD28 for 1 day (2 μg/mL each, clones 145-2C11 and 37.51, respectively, BioXCell). Activated T cells were transduced on day 1 after stimulation using combinations of PEG-precipitated retroviral concentrates encoding different constructs adsorbed onto non-tissue culture treated 6-well plates coated with anti-CD3/CD28 2 μg/mL each and retronectin 20 μg/mL (Takara, T100B) at 1-2×10⁶ cells/well. On day 2, T cells were either re-transduced, or transferred to 6-well plates coated with anti-CD3/CD28. Cells were removed on day 3, Zip-sorted, and expanded in 50 IU/mL rhIL-2 (Proleukin).

### 15.1.5 Target Cell Line Construction

BM185-CD19 was constructed by transducing BM185 cells with LZRS ffluc-Thy1.1-Neo and CD38 was deleted for use in concurrent studies using Cas9-NLS (UC Berkeley MacroLab), CD38 gRNA UAAAUUCAUAGUUAGCCAUU (SEQ ID NO: 123, Synthego), and Lonza SF buffer kit (Lonza, V4XC-2032) with a 4D Nucleofector using code DN100. Subclones were identified that were CD19⁺ CD38⁻. BM185-CD20 was similarly generated as a clone uniformly expressing the LZRS CD20 and LZRS ffluc-Thy1.1-Neo transgenes. CD19 was deleted with CD19 gRNA UGAUUCAAACUGCUCCCCCG (SEQ ID NO: 124) and CD38 deleted with the CD38 gRNA, after which cells were negatively selected for CD19 (CD19 microbeads, Miltenyi, 130-121-301) and CD38 (anti-CD38 PE, anti-PE microbeads, Miltenyi, 130-048-801). BM185-CD19-CD20 expresses LZRS ffluc-Thy1.1-Neo. BM185-CD19-FasL was generated from a BM185-CD19 ffluc-Thy1.1-Neo clone generated for concurrent studies with deletion of CD38 and CD22 (CD22 gRNA UGUCAUUGGCACGUAUCGGG, SEQ ID NO: 125, Synthego) and transduced with LZRS FasL and subcloned. BM185-CD20-PD-L1 and BM185-CD20-CD200 were similarly generated from CD38, CD22-deleted clones modified to express PD-L1 (LZRS PD-L1) or CD200 (PB-EF-1α-intron CD200, pCMV-hyPBase, SF buffer kit V4XC-2032, Lonza 4D Nucleofector, code DN100). Transposition reactions used 1-2 μg of vector DNA and 0.5-1.0 μg of pCMV-hyPBase transposase DNA. These cell lines were then subcloned. C1498-CD19 and C1498-CD20 versions were generated by transduction with SFG CD19 and LZRS CD20 and LZRS ffluc-Thy1.1-Neo and immunomagnetically sorted for high transgene expression. However, long-term retroviral expression in C1498 was unstable, so we generated a stable C1498 CBR-hCD8-puro clone using PB-EF-1α-intron transposon and generated subcloned versions individually transposed with the PB-EF-1α-intron transposon vectors encoding CD19, hCD19, CD20, CD79bΔ, or BAFF-R (transposon, pCMV-hyPBase, SF buffer kit V4XC-2032, Lonza 4D Nucleofector, code DA100). CD79bΔ refers to a chimeric protein comprising mouse CD79b extracellular domain with a CD28TM and truncated CD3ζΔ to promote surface expression in the absence of CD79a. For Incucyte® live-cell microscopy analysis, target cells were transduced with PB-MSPV-puro iRFP713-P2A-hygro-E2A-TAA-WPRE and selected in hygromycin B (Santa Cruz Biotechnology, sc-29067) at 0.8 mg/mL to enable near-infrared imaging.

### 15.1.6 Zip-Sorting

Transduced C1498 or T cells were incubated with anti-FLAG (Miltenyi, 130-101-591) or anti-CD34 (Miltenyi, 130-046-702) beads at 30 μL of beads per 10⁷ cells for 30 minutes at 4 C in PBS 2 mM EDTA+0.5% BSA, washed in PBS 2 mM EDTA+0.5% BSA, centrifuged 1200 RPM×5 minutes and resuspended in 500 μL PBS 2 mM EDTA+0.5% BSA. Cells were sorted on LS columns (Miltenyi, 130-042-401) by washing 3× (3 mL, 2 mL, 1 mL) with PBS 2 mM EDTA+0.5% BSA and eluting with 5 mL T cell media. Zip-sorted T cells were used for in vitro experiments on days 4-6 post-stimulation and injected into mice for in vivo experiments on day 5 post-stimulation.

### 15.1.7 Incucyte® Imaging

T cells were transduced with either PB-SFG5.3-BlastR EGFP (constitutive EGFP), or NFκB reporter vectors (inducible, destabilized EGFP reporter), added at 2×10⁴ cells/well to 96-well flat-bottom plates, and co-cultured with iRFP713-expressing target lines at varying effector:target (E:T) ratios in T cell media without rhIL-2. T cell and tumor cell line fluorescence was imaged simultaneously every 3 hours with 10× objective, 4 images per well, in an Incucyte® SX5 (Sartorius). For stress-test experiments, targets were added back to the plate at 1:1 initial E:T ratio at 24 and 48 hours after initial assay setup. Data were analyzed using Incucyte® Software v2021A.

### 15.1.8 Continuous Live-Cell Microscopy

T cells were transduced with either PB-SFG5.3-BlastR EGFP (constitutive EGFP) or NFκB reporter vectors (inducible, destabilized EGFP reporter), added at 2×10⁴ cells/well to 96-well flat-bottom plates, and co-cultured with iRFP713-expressing target lines at varying E:T ratios in T cell media without rhIL-2. T cell and tumor cell line fluorescence was imaged simultaneously every 3 hours with 10× objective, 4 images per well, in an Incucyte® SX5 (Sartorius). For stress-test experiments, targets were added back to the plate at 1:1 initial E:T ratio at 24 and 48 hours after initial assay setup. For T cell cluster analysis, green fluorescence object threshold was set to minimum of 5000 μm², edge split off, eccentricity maximum 1.0, hole fill 0 μm². Data were analyzed using Incucyte® Software v2021A.

### 15.1.9 Bioluminescence-Based Target Lysis Assay

T cells were incubated in triplicate in 96-well U bottom plates for 24 hours at varying E:T ratios with 0.5-1×10⁴ luciferase-expressing targets in T cell media lacking rhIL-2. A no-T cell row was added to obtain relative maximum luciferase activity. For experimental readout, luciferin (Gold Bio LUCK-2G) was added to wells to achieve final 200 μg/mL concentration and luciferase activity was analyzed on a Tecan SPARK luminometer/fluorimeter (Tecan). Target relative percent viable values were calculated as 100*(experimental well activity units/maximum activity units).

### 15.1.10 Flow Cytometry

Flow cytometry analysis acquisition was performed on an LSR-II or FACSymphony™ X50 using FACSDiva™ software (BD Biosciences). Analysis was performed using FlowJo software (BD Biosciences, version 10.8.1). Cell viability was assessed with DAPI (Calbiochem, 5087410001). MFI refers to geometric mean fluorescence intensity. Intracellular flow cytometry analysis was performed by first antibody staining cells for surface markers and live/dead status using LIVE/DEAD Fixable Blue Dead Cell Stain Kit (Invitrogen, L23105), followed by permeabilization using the Foxp3/Transcription Factor Staining Buffer Set (Invitrogen, 00-5523-00) and antibody stained for intracellular contents. CAR expression was detected by flow cytometry with antibodies against affinity tags including Myc, Streptavidin tag, FLAG, hCD20 mimotope (Rituximab-APC), and the hCD34 tandem epitope with their respective antibodies. Streptavidin tag was also detected using Streptactin-PE for some experiments (Iba Biosciences, 6-5000-001). Rituximab was obtained from the MSKCC pharmacy and APC conjugated (APC Conjugation Kit—Lightning-Link, Abcam, ab201807). Anti-streptavidin tag purified antibody (Genscript) was also APC conjugated. The BAFF-R CAR was stained with 1 μg of hBAFF-R hFc (Sino Biological, 16079-H02H), followed by anti-hFc antibody. Similarly, PD-1-DNR interaction with PD-L1 was assessed by staining with 1 μg Recombinant Mouse PD-L1/B7-H1 Fc Chimera Protein (R&D Systems, 1019-B7-100), followed by anti-hFc antibody.

### 15.1.11 Complement Lysis Assay

T cells were incubated at 5×10⁴ cells/well in a 96-well U bottom plate with Rabbit complement (final concentration 10%, Cedarlane, CL3051)±100 μg/mL anti-Thy1.1 (clone 9E12, BioXCell) for 30 minutes at 37 C. Subsequently, viable cells were enumerated by FACS, measuring DAPI-negative viable cells using CountBright Beads (Invitrogen, C36950). Relative T cell survival was calculated as 100*(viable cells: antibody+complement/viable cells: complement only).

15.1.12 iCaspase9 Activation and Cytokine Release Assay

For dimerizer titration, T cells were incubated at 3×10⁴ cells/well a 96-well U bottom plates for 24 hours in T cell media containing 50 IU/mL rhIL-2 and varying concentrations of AP20187 (B/B homodimerizer, Takara, 635058). After 24 hours, cells were analyzed for DAPI-negative viable cells using CountBright™ Beads (Invitrogen, C36950). Relative T cell survival was calculated as 100*(viable cells: dimerizer/viable cells: DMSO). For assessment of cytokine release following iCaspase9 activation, T cells were incubated at 5×10⁴ cells/well in a 96-well U bottom plates for 24 h in T cell media containing 50 IU/mL rhIL-2 and 100 nM AP20187 or DMSO was added. At 24 hours supernatants were obtained and IL-12 secretion was assessed via IL-12 cytometric bead array kit (BD Biosciences, 560151).

### 15.1.13 Reactive Oxygen Species Assessment

Unstimulated day 5 T cells were plated at 5×10⁴ cells/well in 96-well U bottom plates, washed once with 200 μL of PBS, resuspended in 100 μL of PBS containing 1 M CM-H₂-DCFDA (Invitrogen, C6827) or 5 M Mitosox™ Red (Invitrogen, M36008) for 30 minutes at 37 C. Cells were washed twice in 200 μL of PBS+0.5% BSA, stained with anti-CD4/CD8, and analyzed by FACS for DAPI-negative viable cells.

### 15.1.14 CFSE Dilution

T cells were stained with 1 μM carboxyfluorescein succinimidyl ester (CFSE, BioLegend, 423801) and added at 2×10⁴ cells/well in 96-well U bottom plates containing 80 cGy-irradiated targets at a 1:3 E:T ratio without IL-2. Three days later, T cells were analyzed by FACS for DAPI-negative Thy1.2⁺ T cells and cell counts per well were determined using CountBright Beads (Invitrogen, C36950).

### 15.1.15 Transcription Factor Reporter Assay

T cells were transduced with transcription factor reporter vectors on day 1 post-stimulation (to ensure equivalent reporter transduction among constructs), on day 2 cells were transduced with CAR construct vectors and Zip-sorted or left unsorted for further analysis. T cells were assessed for EGFP induction by FACS following culture with or without target cells in BFP-transduction reporter-positive cells.

### 15.1.16 In Vivo Experiments

Female BALB/cJ (Jackson Laboratory, 00651) and B6(Cg)-Tyrc-2J/J albino B6 (Jackson Laboratory, 000058) mice were purchased at 6-8 weeks of age and used in experiments at 7-12 weeks of age. CD45.1 congenic BALB/c mice (Jackson Laboratory, 006584) were bred at MSKCC. Animal studies were conducted in the MSKCC vivarium under a protocol approved by the MSKCC Institutional Animal Care and Use Committee. Mice were evaluated at least twice daily by MSKCC veterinary staff and lab members and euthanized when reaching any of the following humane endpoints: weight loss >25%, labored breathing, moribund status, hind-limb paralysis, development of ascites, tumor >2 cm, or interfering with bodily functions.

### 15.1.17 BM185 Pre-B ALL Mouse Model

BALBc/J mice were sublethally irradiated with 450 cGy of gamma radiation (Gammacell, cesium source), rested for 4 hours, and then injected with varying doses of BM185 cell lines via tail vein in 200 μL of DMEM without additives (day 0). Mice were randomized into groups following leukemia injection. On day 2, Zip-sorted T cells were injected into the retroorbital plexus in 150 μL of DMEM. T cell and BM185 cell doses are depicted in figures above survival or BLI plots. Mice were evaluated daily for evidence of reaching humane endpoints described in the In vivo experiments section. Mice were serially assessed for leukemia progression via firefly luciferase (ffluc)-based BLI. In some experiments, spleens or bone marrow (BM) were obtained at the time of euthanasia for further analysis. Spleens and BM were dissociated through 40-micron filters, red blood cell lysed (Hybri-Max™, Sigma, R7757), and stained with anti-CD3ε, Thy1.1, CD19, and CD20. Spleens or BM with <0.1% Thy1.1⁺ CD3⁻ BM185 cells or <10 events were excluded from analysis of BM185 surface phenotype. In some experiments, mice were bled via retroorbital plexus, blood was red blood cell-lysed, and stained for flow cytometry analysis.

### 15.1.18 C1498 Acute Myeloid Leukemia Model

Albino B6 mice were sublethally irradiated with 550 cGy of gamma radiation (Gammacell, cesium source), rested for 4 hours, and then injected with varying doses of C1498 cell lines via tail vein in 200 μL of DMEM without additives (day 0). Mice were randomized into groups following leukemia injection. On day 2, Zip-sorted T cells were injected into the retroorbital plexus in 150 μL of DMEM. T cell and C1498 cell doses are depicted in figures above survival or BLI plots. Mice were evaluated daily for evidence of reaching humane endpoints described in the In vivo experiments section. Mice were serially assessed for leukemia progression via CBR luciferase based BLI. In some experiments, BM was obtained at the time of euthanasia for further analysis. BM was dissociated through 40-micron filters, red blood cell lysed (Hybri-Max™, Sigma, R7757), and stained for hCD8 and tumor target antigens. BM with <0.1% hCD8⁺ C1498 cells or <10 events was excluded from C1498 surface phenotype analysis. In BM of some CAR T cell treated mice, we observed an amorphous debris that simultaneously stained positively for all flow markers: hCD8, CD19, CD20, CD79b, and BAFF-R. This population was gated out of analyses. The C1498 model was less predictable than BM185, with mice sometimes dying overnight despite looking otherwise healthy on the prior night. Additionally, a subset of CAR T cell treated mice apparently cleared leukemia from the BM with extramedullary progression. Therefore, the number of available BM samples with C1498 to assess for antigen-loss escape was diminished compared with the BM185 model.

### 15.1.19 Bioluminescence Imaging (BLI)

For serial quantitative assessment of leukemia, mice were injected with D-luciferin (Gold Bio, LUCK-2G) at 150 mg/kg dose intraperitoneally. Ten minutes after injection, isoflurane-anesthetized mice were imaged using an IVIS Spectrum CT imaging system (PerkinElmer). For serial quantitative assessment of T cells, mice were injected with 100 g of water-soluble Coelenterazine (NanoLight Technology, 3031) into the retroorbital plexus and imaged immediately. Mice were imaged individually following injection.

### 15.1.20 Statistical Analysis

For BLI curves, groups were compared using area under the curve (AUC) analysis performed on log-transformed BLI values using a Vardi test with the function aucVardiTest in the R package clinfun. False discovery rate (FDR) correction was applied to account for multiple comparisons. Pairwise log-rank tests were performed by the function pairwise_survdiff in the R package survminer, followed by FDR correction for multiple tests. All other statistical tests were performed using GraphPad Prism, with test type described in figure legends.

### 16.2 Results

### 15.2.1 Exemplary Approaches for Constitutive Inhibition of NFAT Signaling to Regulate T Cell Function

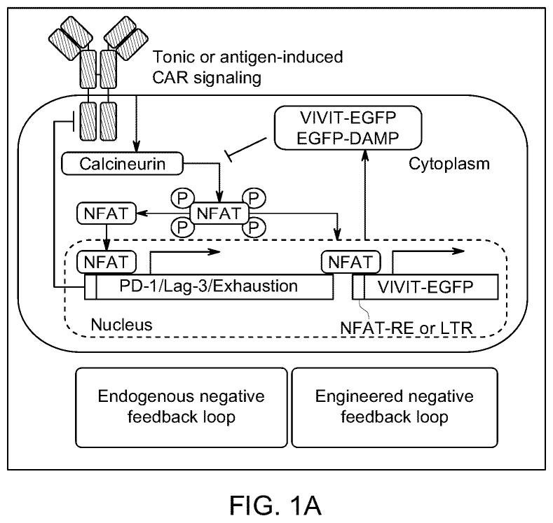

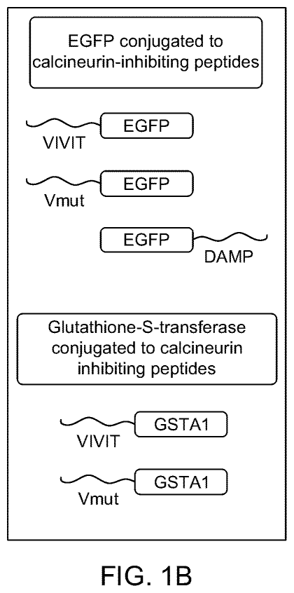

An exemplary approach for inhibiting transcription factor NFAT activation by calcineurin in a CAR expressing T cell is shown in FIG. 1A. This approach uses a VIVIT peptide appended to a “carrier” protein (e.g., enhanced green fluorescence protein) and expressed as a VIVIT-carrier fusion protein. Binding of the VIVIT peptide motif in the fusion protein to calcineurin inhibits the ability of calcineurin to dephosphorylate NFAT. As a result, dephosphorylation and nuclear translocation of NFAT is inhibited, thereby inhibiting induction of proteins involved in cell exhaustion and/or cell death (e.g., PD-1/L1, Lag-3, CD200/R, Tim-3, Fas). FIG. 1B provides exemplary peptides that can be used to inhibit NFAT signaling. In one example, cells can be modified to express VIVIT, variants of VIVIT (Vmut), or other conserved PxIxIT calcineurin binding sequences of NFAT. These VIVIT, Vmut or PxIxIT sequences sequester endogenous calcineurin thereby blocking its interaction with endogenous NFAT, thus effectively inhibiting NFAT signaling. These inhibitory peptides can be expressed as a fusion protein, appended to the C-terminal or N-terminal ends of a suitable carrier protein such as for example, enhanced green fluorescence protein (EGFP) and glutathione-S-transferase (GSTA1), among others. In another example, cells can be modified to express a peptide derived from the autoinhibitory domain of calcineurin (ITSFEEAKGLDRINERMPPRRDAMP), denoted here as “DAMP” can also be used to inhibit calcineurin dependent NFAT signaling. Similar to the PxIxIT group of peptides, the DAMP group of peptides can also be expressed as a fusion protein, appended to the C-terminal or N-terminal ends of carrier proteins like EGFP, GSTA1, or others Thus, overexpression of carrier proteins fused to PxIxIT, or DAMP group of peptides provide constitutive NFAT inhibition to attenuate NFAT signal strength.

### 15.2.2 Exemplary Approaches for Drug-Regulatable Inhibition of NFAT Signaling to Regulate T Cell Function

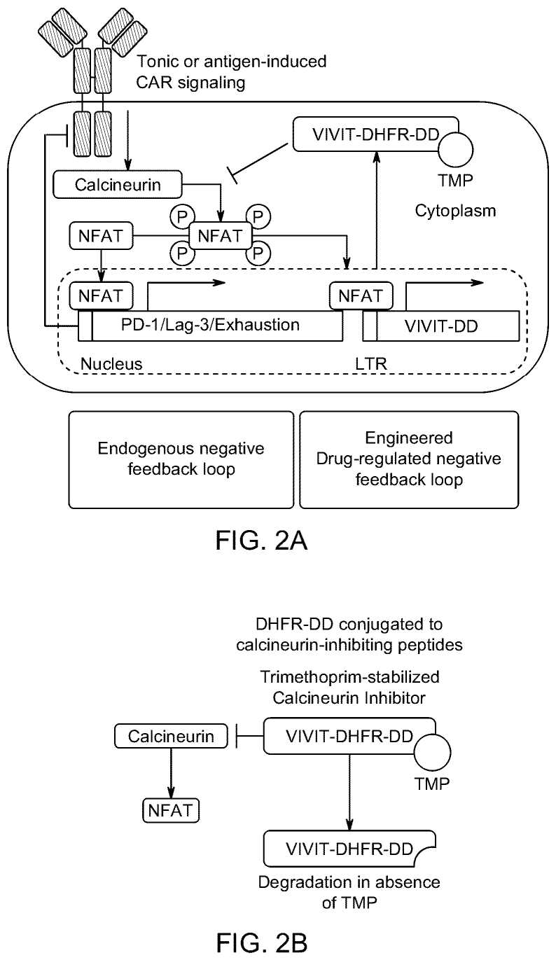

Another approach for inhibiting NFAT signaling is through a drug regulatable expression of PxIxIT, or DAMP peptides, which allows one to control when the inhibitors are being stably expressed in the cells. One illustrative example uses destabilized mutants of dihydrofolate reductase (DHFR-DD) fused to the NFAT inhibitory peptides described above in FIG. 1B. These inhibitory peptides are stably expressed as a Peptide-DHFR-DD fusion protein only in presence of a stabilizing drug (FIG. 2A), for example, trimethoprim (TMP), thereby allowing for drug regulated inhibition of NFAT and thus, drug regulated inhibition of proteins involved in cell exhaustion and/or cell death. Similarly destabilized mutants of FK binding protein (FKBP-DD) can also be used to generate inhibitory peptide-FKBP-DD fusion proteins, stable expression of which require a stabilizing drug, for example, Shield-1 (Shld1). With either DHFR-DD or FKBP-DD, absence of the stabilizing drug results in degradation of the DHFR-DD/NFAT inhibitory peptide or FKBP-DD/NFAT inhibitory peptide (FIG. 2B) thus allowing control of NFAT signaling in the transduced cell.

### 15.2.3 Exemplary Promoters for Selective Expression of NFAT Inhibitors

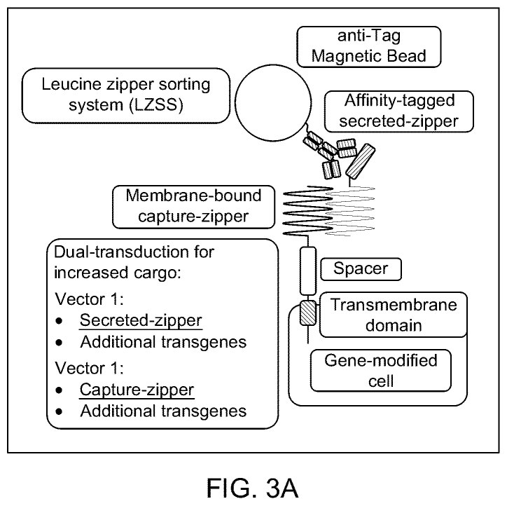

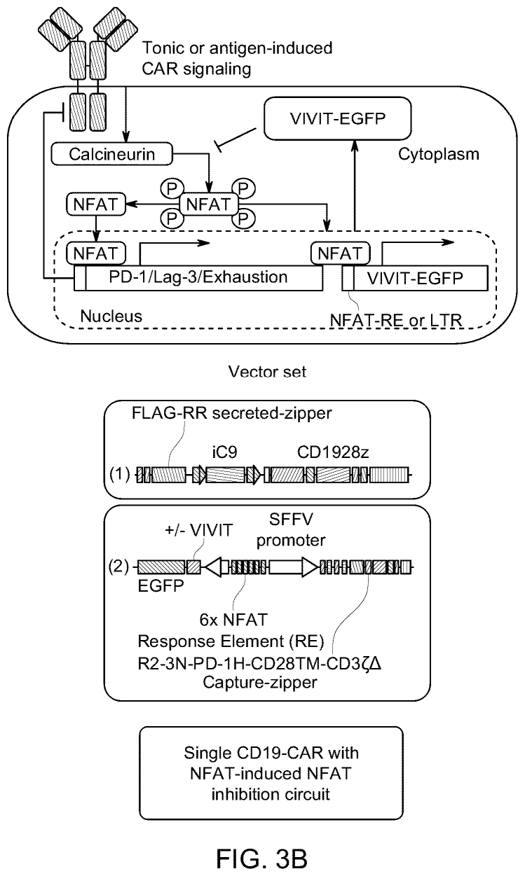

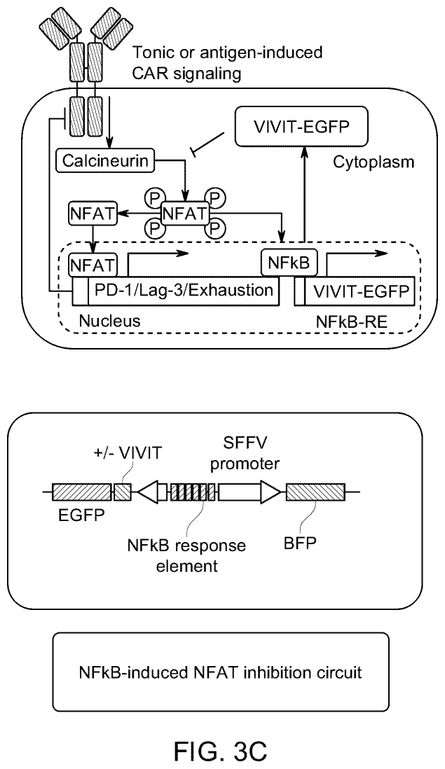

FIG. 3B is a vector map illustrating use of an NFAT responsive promoter (NFAT-response element) to drive NFAT-dependent expression of the inhibitory peptide-carrier fusion proteins (e.g., VIVIT-EGFP). The use of an NFAT-response element provides for a negative feedback loop that inhibits excessive NFAT signaling in the transduced cells (e.g., T cells) when encountering targets with high antigen expression for extended durations, while avoiding NFAT inhibition against weaker targets that induce NFAT to a lesser extent. A similar approach using a NFkB responsive promoter to drive NFkB-dependent expression of the inhibitory peptide-carrier fusion proteins is illustrated in FIG. 3C.

### 15.2.4 Leucine Zipper Sorting Systems Facilitate Production of T Cells Expressing NFAT Inhibitors

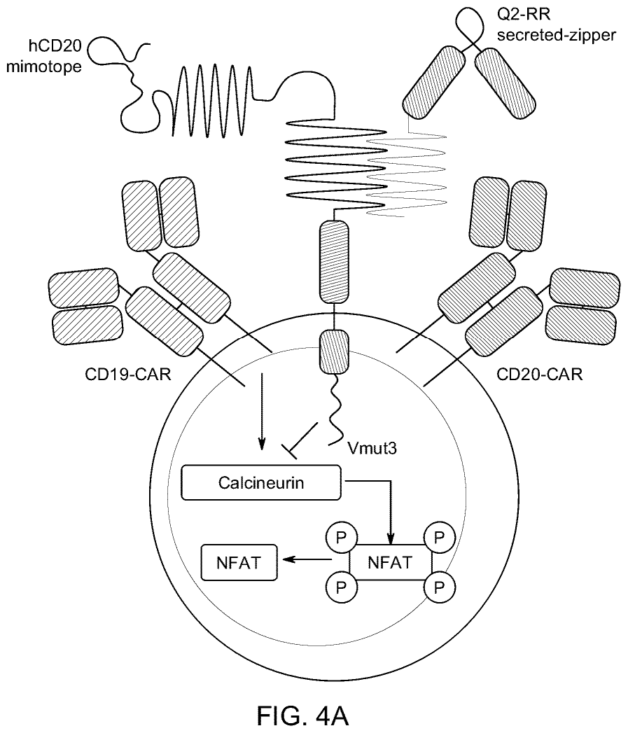

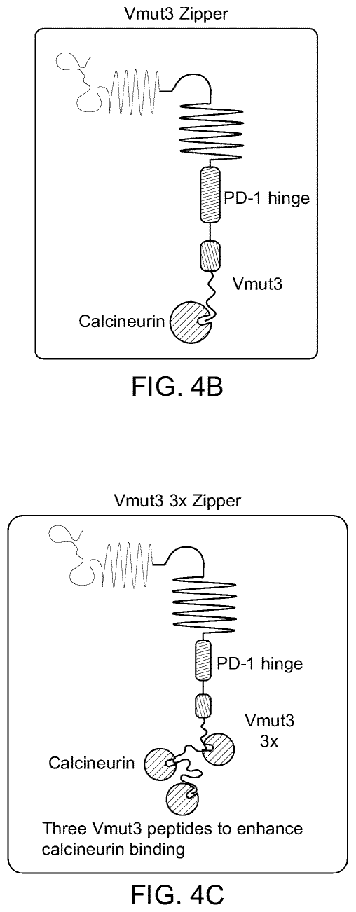

In yet another exemplary approach the calcineurin/NFAT signaling inhibitory peptides are expressed as a fusion protein attached to the cytoplasmic-facing domain of a capture zipper sequence comprising a transmembrane sequence, which heterodimerizes with a zipper sequence that is secreted outside the cell (secreted zipper, FIG. 4A). Use of zipper-based systems permits zip sorting of transduced cells to obtain highly purified cells that express the calcineurin/NFAT inhibitory peptides. Optionally, the capture zipper may have a CD20 mimotopes attached on their extracellular domain. FIG. 4B shows one variant of the zipper-based sorting system in which the capture zipper comprises an extracellular human CD20 mimotope, a leucine zipper domain, a PD-1 hinge domain, a transmembrane domain and an intracellular domain to which is attached a calcineurin/NFAT signaling inhibitory peptide (e.g., Vmut3). FIG. 4C illustrates another variant of the zipper-based sorting system in which the capture zipper comprises an extracellular human CD20 mimotope, a leucine zipper domain, a PD-1 hinge domain, a transmembrane domain and an intracellular domain to which is attached three calcineurin/NFAT signaling inhibitory peptides (e.g., 3×Vmut3).

### 15.2.5 Attenuating NFkB Signaling in CAR T Cells

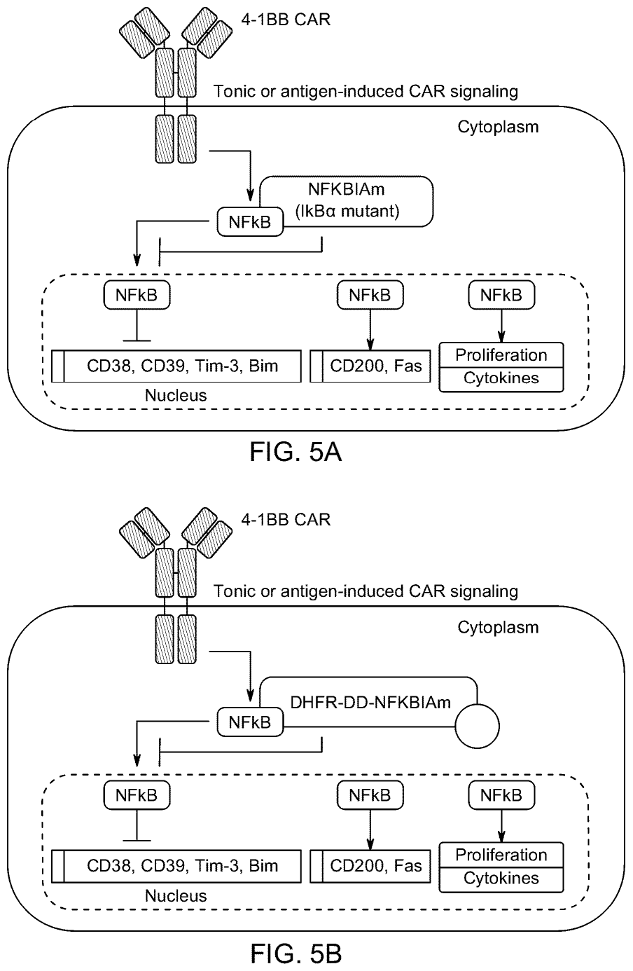

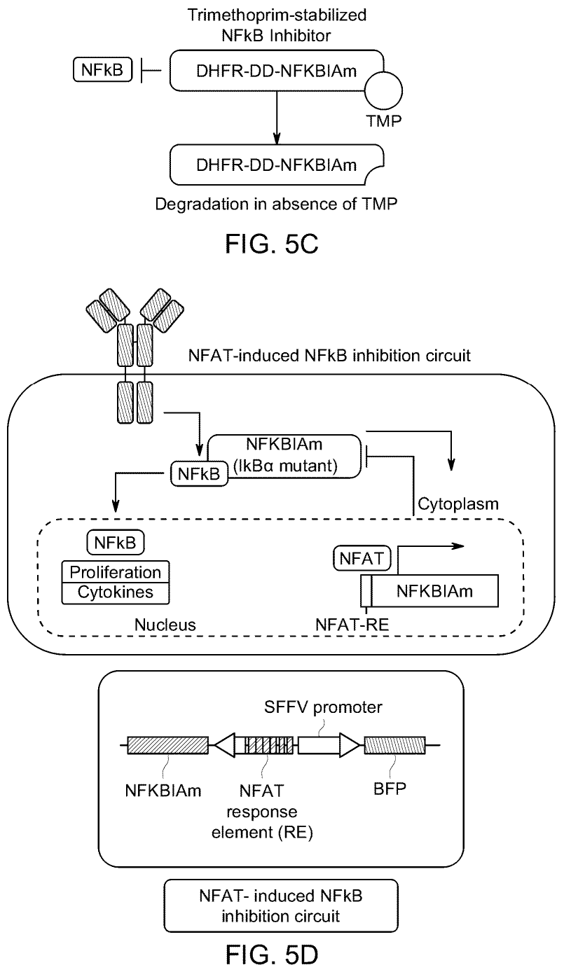

Another approach for regulate T cell function is through attenuation of NFkB signaling. One example of such an approach is illustrated in FIG. 5A, which shows modifying T cells to express degradation-resistant mutants of NFkB (e.g., NFKBIAm also known as IkBαm) to regulate T cell survival, proliferation, and cytokine production. The cells can also be transduced with drug-regulatable NFKBIAm construct, for e.g., NFKBIAm fused to destabilized mutants of dihydrofolate reductase (DHFR-DD) to enable drug-regulated NFkB attenuation (FIGS. 5B, 5C) as discussed in Section 16.2.2. Destabilized mutants of FK binding protein (FKBP-DD) can similarly be used to generate cells expressing NFKBIAm-FKBP-DD fusion proteins. As discussed in Section 16.2.3, constitutive or drug regulatable expression of NFKBIAm can be driven using for example NFkB or NFAT responsive promoters (FIGS. 5A-5D), which provide a negative feedback loop to regulate excessive NFKBIAm expression in the transduced cells.

### 15.2.6 Drug Regulated Attenuation of NFAT Signaling

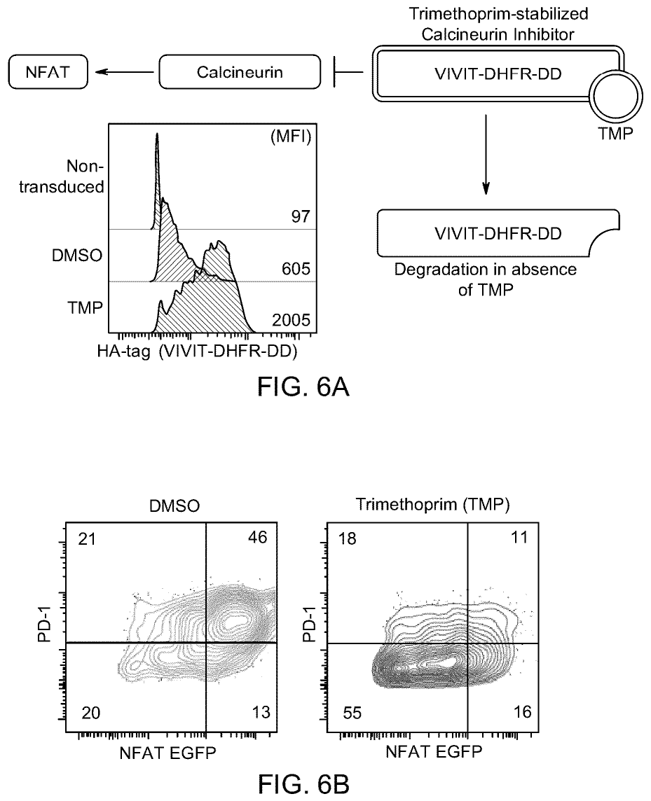

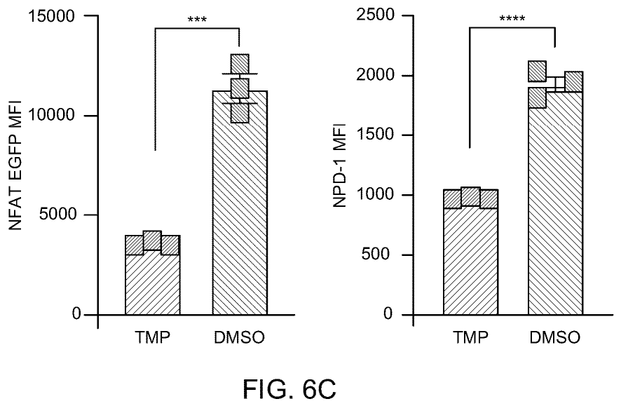

To test the effects of TMP inducible VIVIT peptide expression, T cells were transfected with hemagglutinin tagged (HA-tag) VIVIT-DHFR-DD. FACS analysis revealed a TMP dependent expression of VIVIT-DHFR-DD in the T cells as visualized by HA-tag staining. T cells transduced with vectors encoding VIVIT-DHFR-DD and NFAT-EGFP were analyzed for PD-1 and NFAT-EGFP expression by FACS. The data in FIG. 6B shows that expression of PD-1 is reduced in the NFAT-EGFP positive cells that are cultured with TMP, demonstrating the benefit of using drug regulatable VIVIT expression to reduce expression of T cell inhibitory molecules including PD-1. A quantitative assessment of NFAT-EGFP expression in the absence or presence of TMP is shown in FIG. 6C.

### 15.2.7 Constitutive Attenuation of NFAT Signaling in Zip-Sorted CAR T Cells

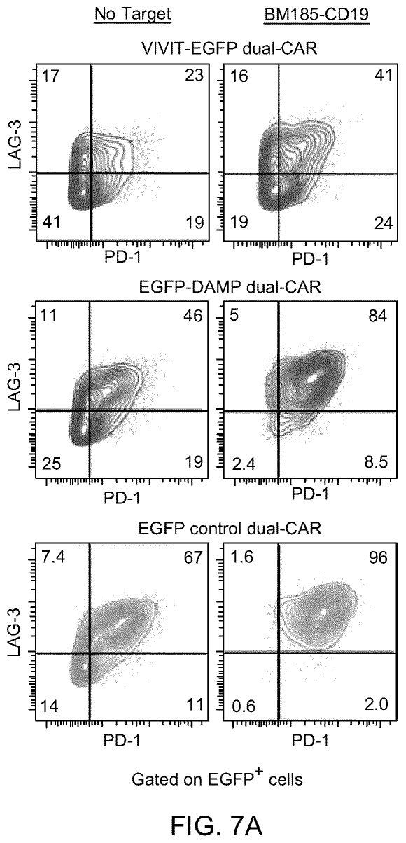

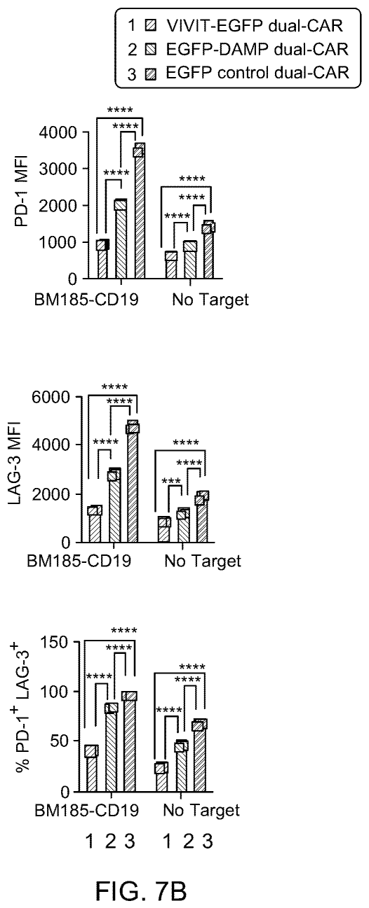

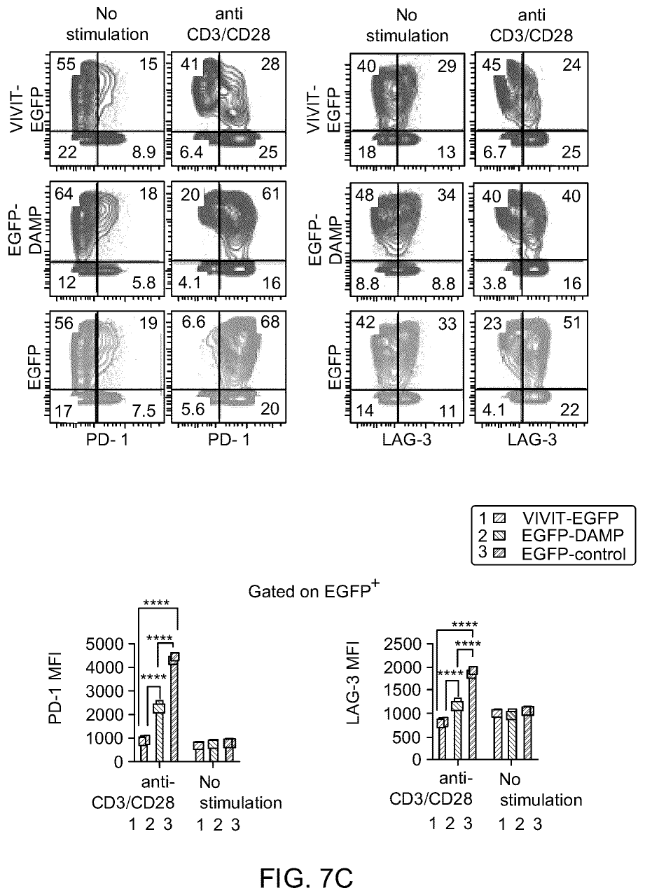

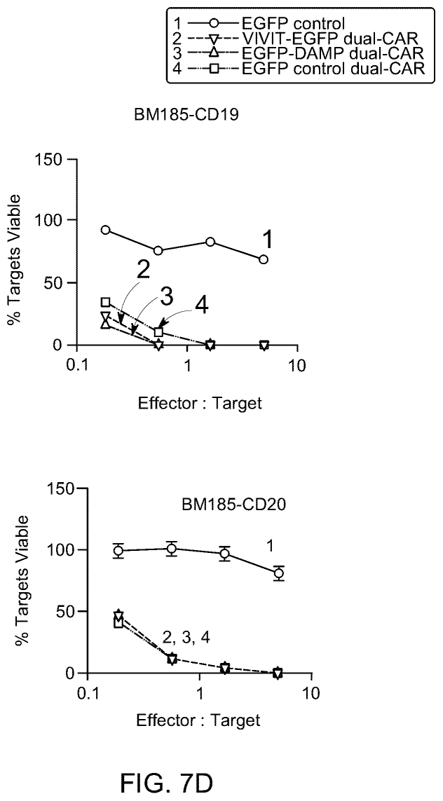

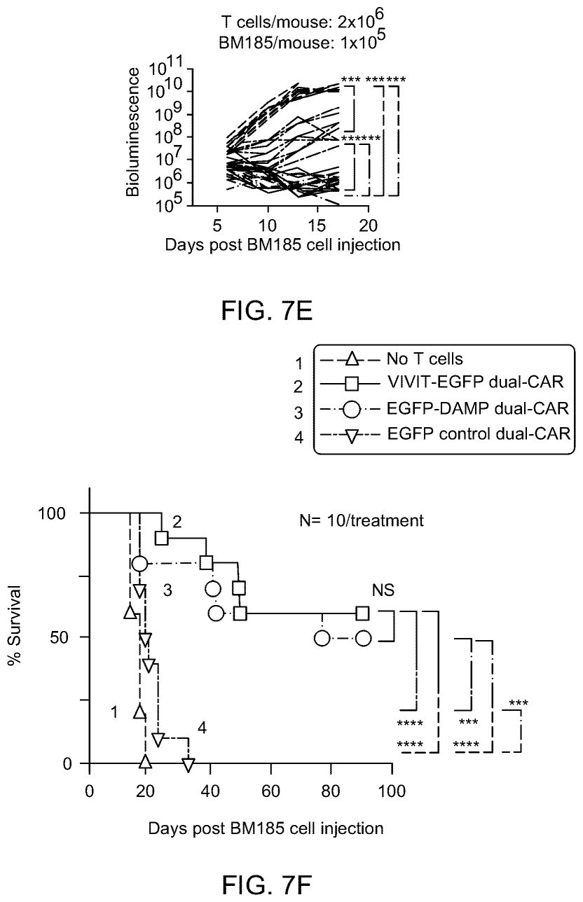

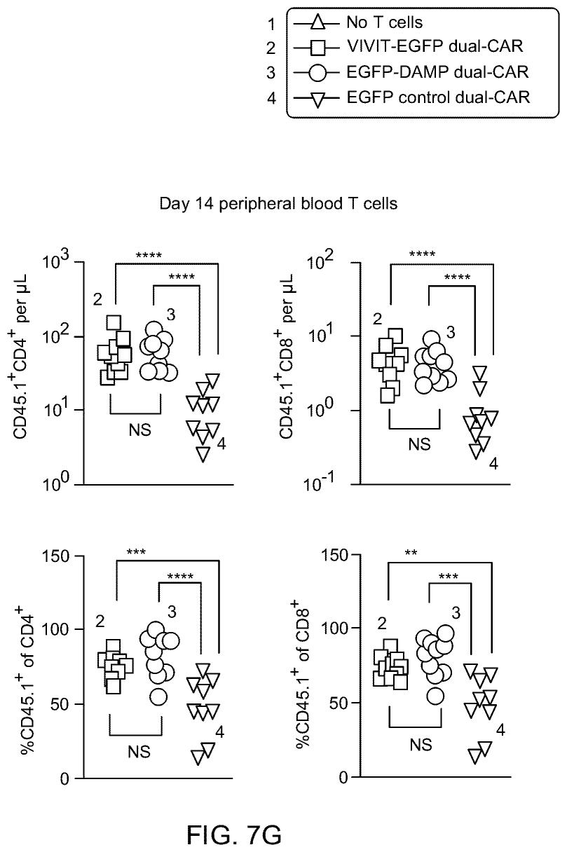

In vitro Assay: Zip-sorted CD19-28z/CD20-28z dual-CAR T cells (see FIG. 4A) were transduced with VIVIT-GFP, EGFP-DAMP, or EGFP constructs. The transduced cells were cultured for 24 h in the absence or presence of CD19 expressing BM185 target cells followed by FACS analysis for expression of T cell exhaustion markers PD-1 and LAG-3. As seen in FIG. 7A, expression of PD-1 and LAG-3 is reduced by constitutive inhibition of NFAT signaling in CAR T cells expressing VIVIT or DAMP. Quantitative assessment of PD-1 and LAG-3 expression is shown in FIG. 7B. Zip-sorted CD19/28z/CD20/28z dual-CAR T cells were stimulated for 24 h with anti-CD3/CD28 and assessed for PD-1 and LAG-3 expression. FIG. 7C shows upregulation of PD-1 and LAG-3 upregulation in response to anti-CD3/CD28, which is reduced in dual CAR_T cells that co-express NFAT signaling inhibitors. Next, the ability of the dual CART cells to lyse target cells was examined using a luciferase-based target cell lysis assay. FIG. 7D shows loss of target cell viability for both CD19 and CD20 expressing targets.

In vivo Assay: BALB/c mice were injected with luciferase expressing BM185 leukemia followed by administration of the Zip-sorted CD19/28z/CD20/28z dual-CAR T cells transduced with the various constructs. The number of resident leukemia cells in the animals was followed for up to 20 days post injection by bioluminescent imaging (FIG. 7E). FIG. 7F shows the results of survival studies in these animals. Taken together these data show improved survival of tumor bearing mice that were injected with dual CAR cells co-expressing NFAT signaling inhibitors. An increased persistence of dual-CAR T cells co-expressing the NFAT signaling inhibitors was also observed (FIG. 7G).

### 15.2.8 Constitutive Attenuation of NFAT Signaling by VIVIT Mutants

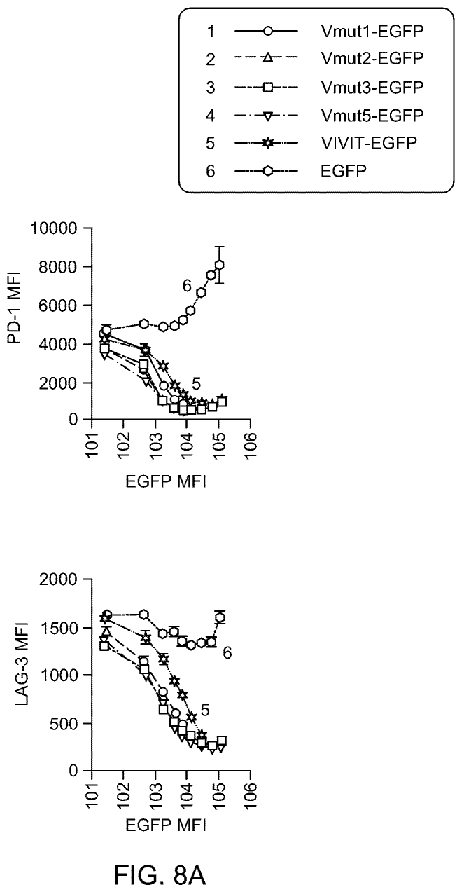

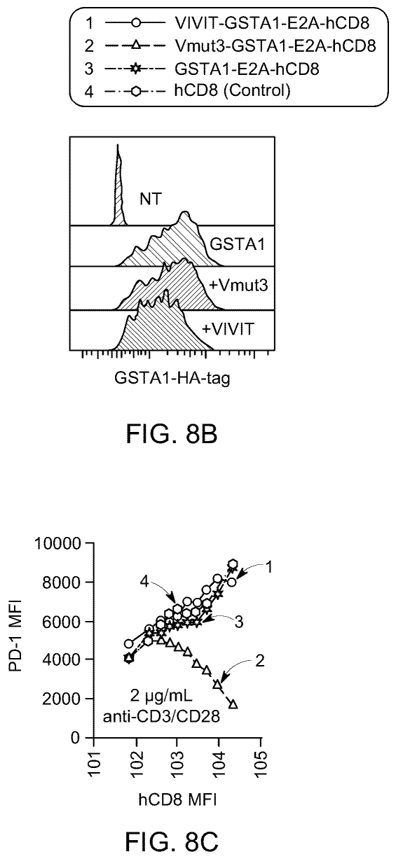

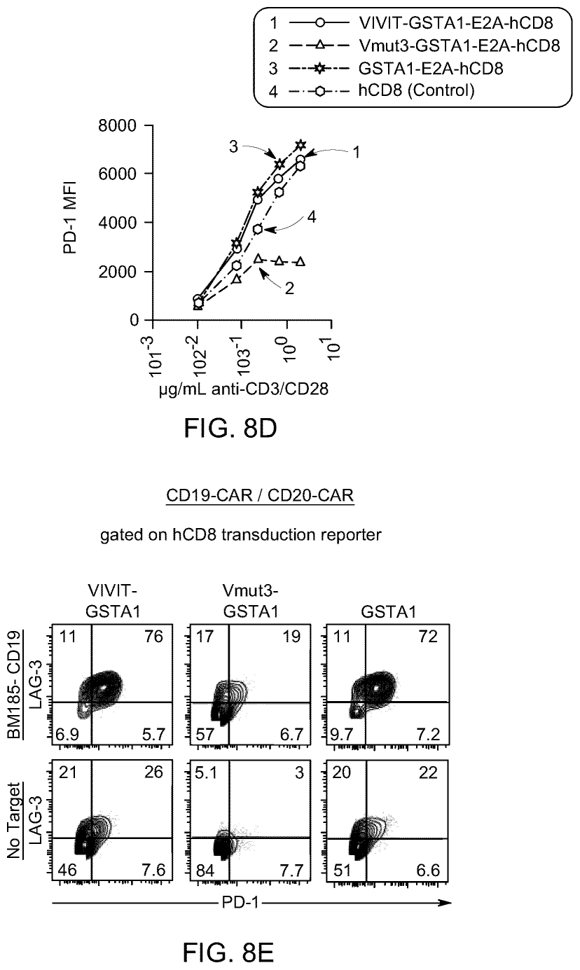

To test the efficacy of VIVIT mutant peptides on PD-1 and LAG-3 down regulation, T cells transduced with vectors encoding these peptides for expression as EGFP fusion peptides. FIG. 8A shows PD-1 and LAG-3 down regulation after 24 h stimulation with anti-CD3/CD28. FIG. 8B shows HA-tag staining in T cells transduced with vectors encoding HA-tagged human GSTA1, VIVIT-GSTA1, and Vmut3-GSTA1. FIG. 8C shows PD-1 upregulation as function of hCD8 reporter expression in T cells transduced with the indicated vectors and stimulated for 24 h with CD3/CD28. FIG. 8D shows PD-1 levels as function of anti-CD3/CD28 in T cells transduced with the indicated vectors and stimulated for 24 h. FIG. 8E shows PD-1 upregulation in T cells transduced with the indicated vectors and stimulated for 24 h in the presence or absence of BM185-CD19 targets.

### 15.2.9 Inducible Inhibition of NFAT Signaling

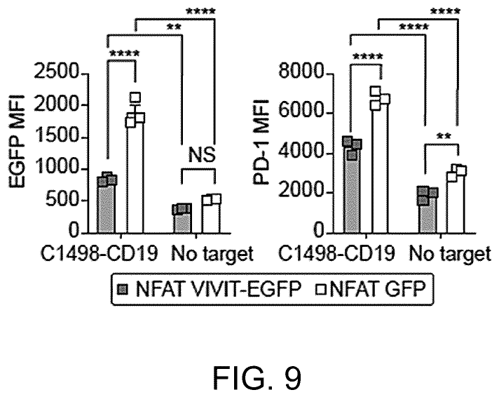

CD1928z CAR T cells were transduced with vectors encoding for EGFP or VIVIT-EGFP under the control of an NFAT responsive promoter (see illustrations in FIGS. 1A and 3B). FIG. 9 shows FACS analysis of EGFP and PD-1 expression in transduced T cells incubated for 2 d with or without C1498-CD19 targets added every 24 h (E:T 1:1, two additions). The NFAT-inducible negative feedback loop is designed to only inhibit NFAT signaling under conditions of strong CAR/TCR activation associated with strong NFAT signaling.

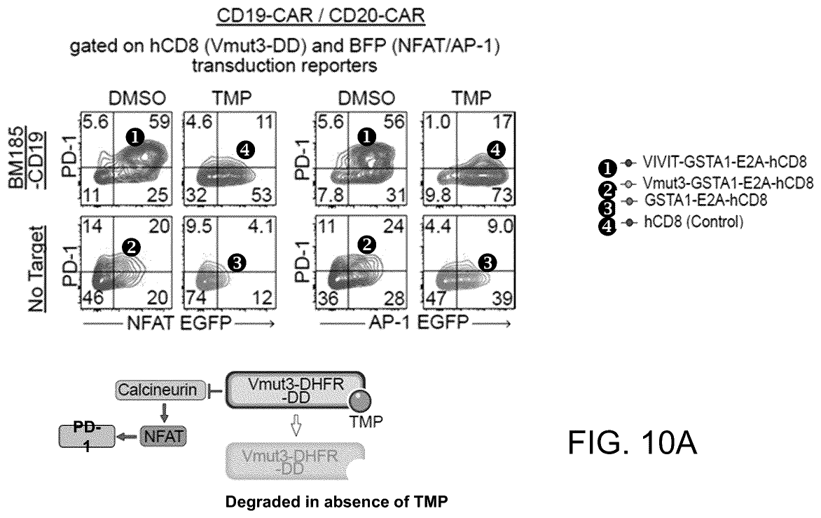

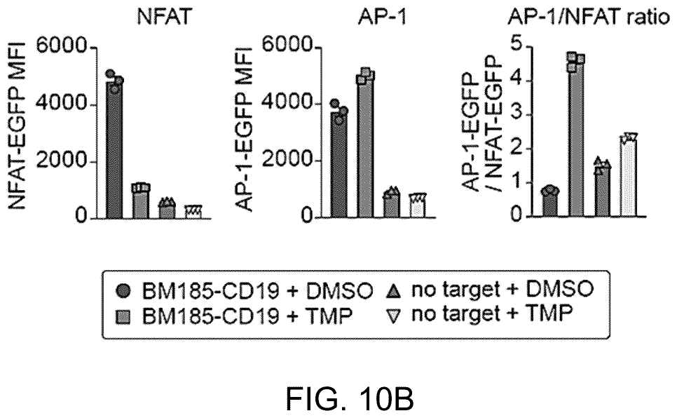

Zip-sorted CD19-28z/CD20-28z dual-CAR T cells were transduced with Vmut3-DHFR-DD-E2A-hCD8 vector. The cells were then cultured for 2 d with TMP and stimulated for 24 h with BM185-CD19 cell targets. FIGS. 10A and 10B show drug-dependent inhibition of NFAT and AP-1 signaling as revealed by reduced PD-1 and LAG-3 expression.

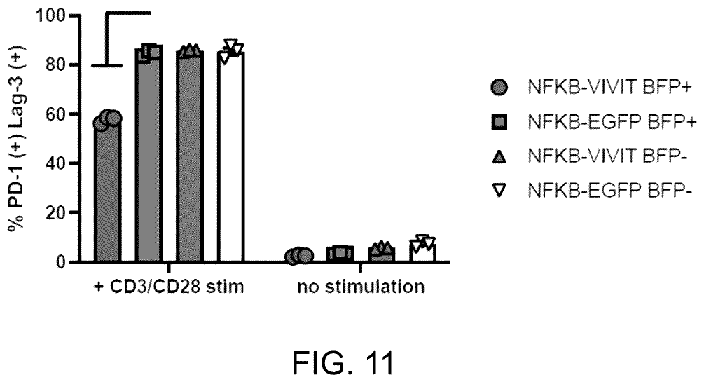

FIG. 11 shows exemplary use of NFkB-inducible NFAT-inhibitor expression (see FIG. 3C) for inhibiting PD-1 and LAG-3 upregulation in transduced T cells stimulated for 24 h with plate-bound CD3/CD28 (2 μg/mL each). PD-1 and LAG-3 expression was inhibited in cells expressing the negative feedback NFkB-VIVIT-EGFP vector (BFP⁺), but not in non-transduced cells (BFP⁻) or control NFkB-EGFP vector.

Conclusion: TMP-regulated Vmut3-DHFR-DD construct inhibited both tonic and antigen-induced NFAT signaling and PD-1 upregulation, and boosted AP-1 signaling, producing an enhanced AP-1/NFAT activation ratio. The NFAT-induced negative feedback loop utilizing VIVIT-EGFP designed with the goal of providing graded NFAT inhibition in response to CAR-induced NFAT signal strength inhibited itself (reduced EGFP induction), and also inhibited tonic and antigen-induced upregulation of PD-1.

### 15.2.10 Leucine Zipper Sorting System for Presenting Calcineurin/NFAT Inhibitory Peptides

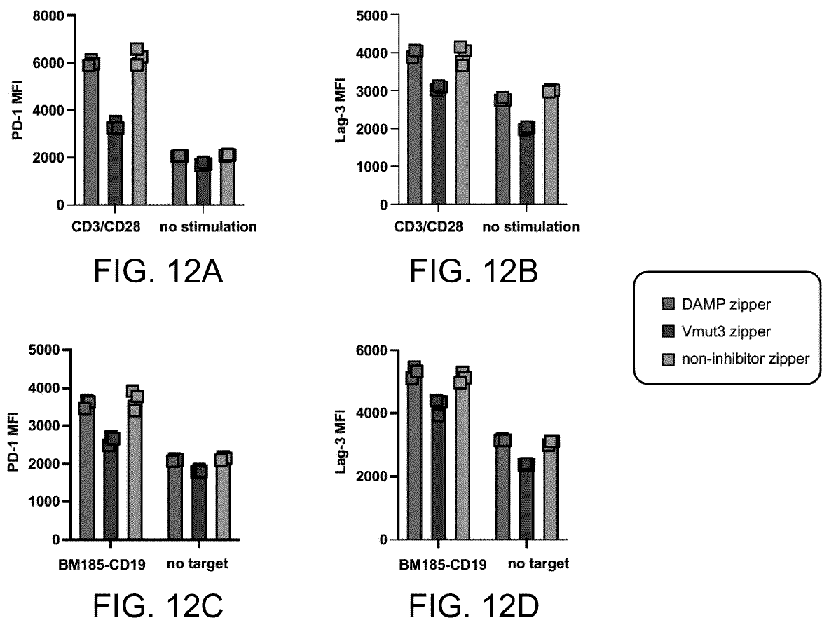

NFAT signaling inhibitory peptide Vmut3, or DAMP conjugated to the C-terminus of a capture-zipper (cytoplasmic side) of a leucine zipper sorting system were expressed in CD19-28z/CD20-28z dual-CAR T cells. Expression of the Vmut3 peptide inhibited CD3/CD28 stimulated (2 μg/mL each, 24 h) upregulation of PD-1 (FIG. 12A) and Lag-3. (FIG. 12B).

Next, the cells were co-cultured for 24 h with BM185-CD19 targets. FIGS. 12C and 12D shows PD-1 and Lag-3 expression respectively following co-culturing the transduced dual-CAR T cells with BM185-CD19 targets. Vmut3, but not DAMP-conjugated capture-zipper inhibited upregulation of PD-1 and Lag-3. Based on these data it is concluded that the relatively lower affinity of the DAMP capture-zipper was insufficient to inhibit antigen or antibody-stimulated inhibitory receptor expression, whereas the higher affinity Vmut3 capture-zipper attenuated inhibitory receptor upregulation.

15.2.11 Effect of Expressing Fusion Proteins with Repeated Inhibitory Peptides on PD-1 And LAG-3

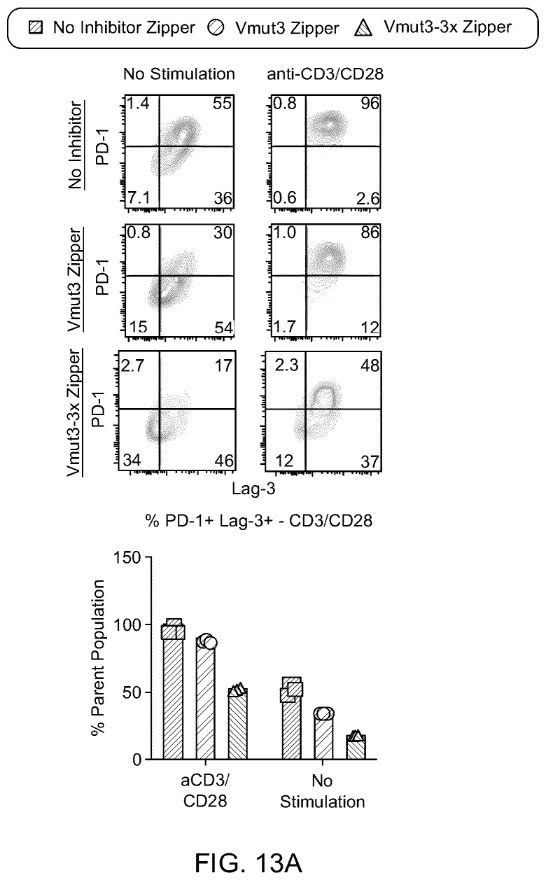

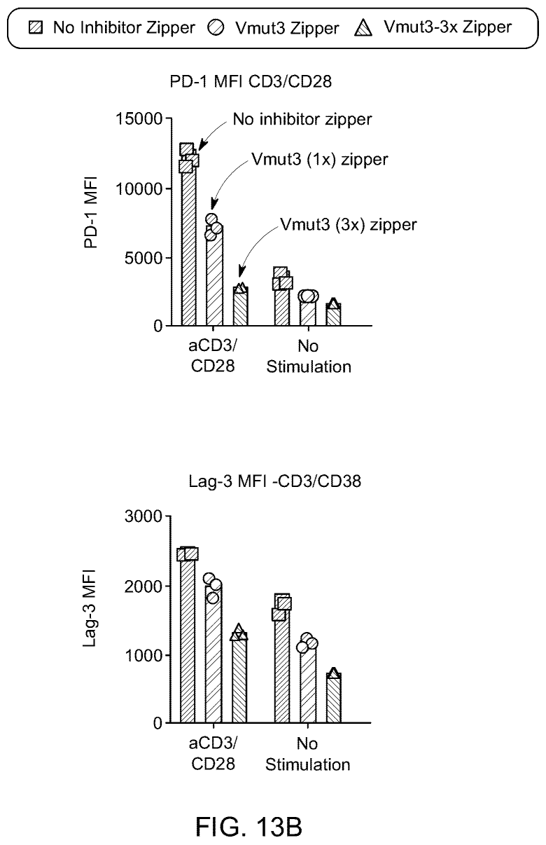

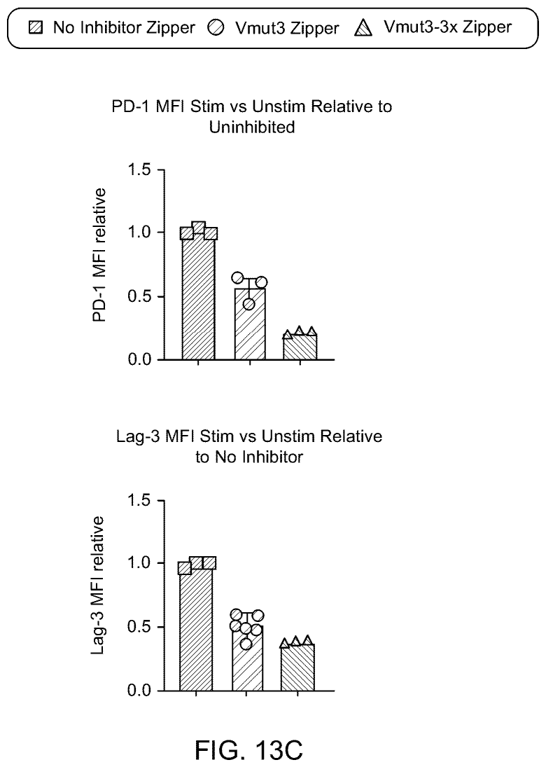

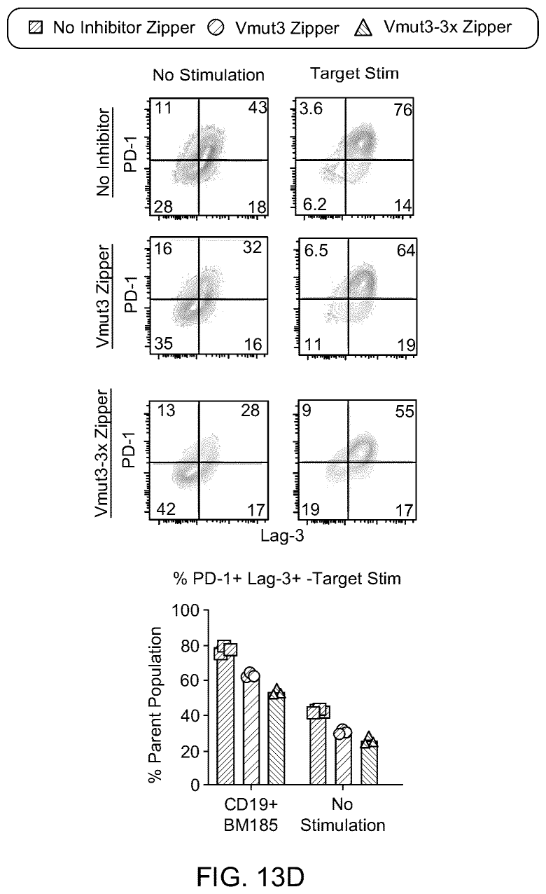

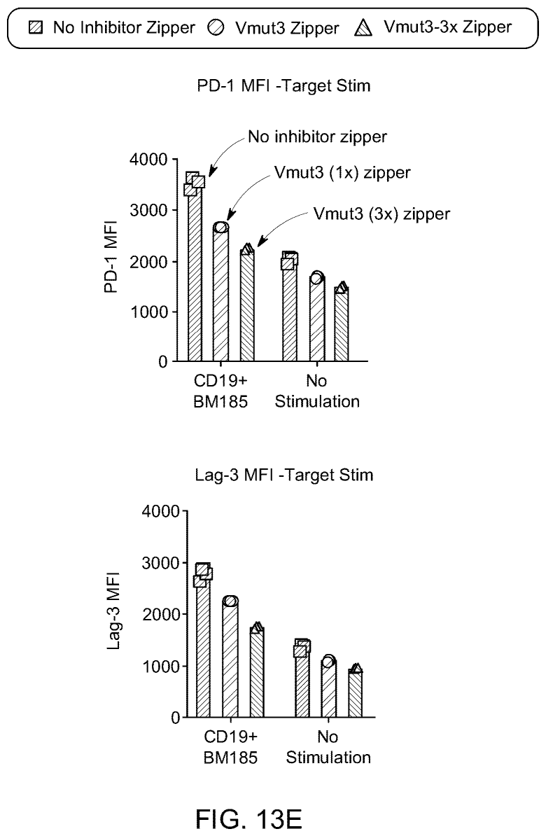

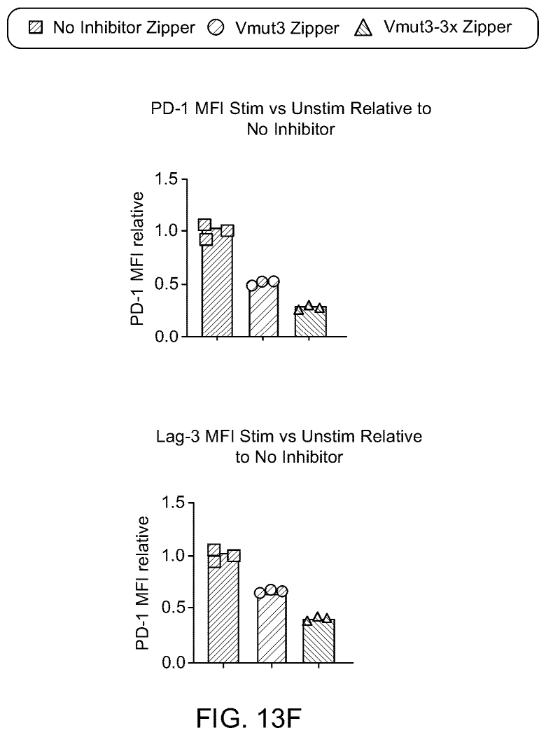

CAR T cells expressing triplet repeats of calcineurin/NFAT inhibitory peptides using the capture-zipper format (see FIG. 4A) were tested for their ability to enhance inhibition of CAR and TCR-stimulated upregulation of PD-1 and Lag-3. FIG. 13A shows FACS analysis for inhibition of anti-CD3/CD8 activated PD-1 and Lag-3 induction in Vmut3 (1×) and Vmut3 (3×) expressing T cells. FIG. 13B shows mean fluorescence intensity (MFI) of PD-1 and Lag-3 expression in the cells described in FIG. 13A. FIG. 13C shows relative inhibition of anti-CD3/CD28 stimulated PD-1 and Lag-3 induction by Vmut3 and Vmut3 3× capture-zippers.

To test the impact on NFAT signaling following stimulation with BM185-CD19 targets, the Vmut3 (1×) and Vmut3 (3×) expressing CAR T cells were incubated for 24 h with the target cells. FIG. 13D shows FACS analysis showing inhibition of PD-1 and Lag-3 induction in these T cells. FIG. 13E shows MFI of PD-1 and Lag-3 in the cells described in FIG. 13D. FIG. 13F shows relative inhibition of antigen stimulated PD-1 and Lag-3 induction by Vmut3 (1×) and Vmut3 (3×) capture-zippers.

### 15.2.12 Vectors-Encoding NFkB Inhibitory Protein NFKBIAm for Inhibiting Tonic Signaling in Dual-CAR T Cells

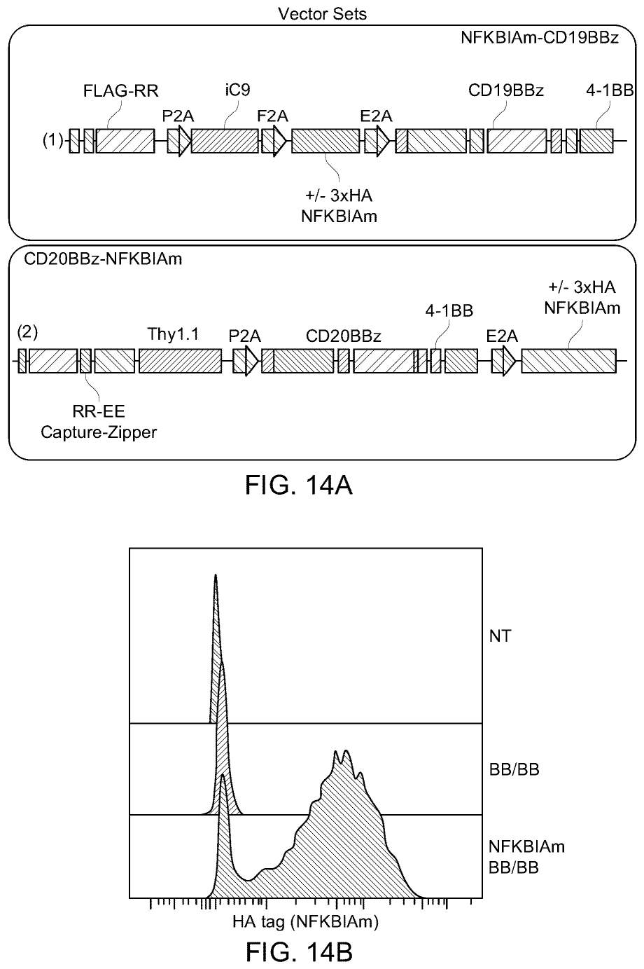

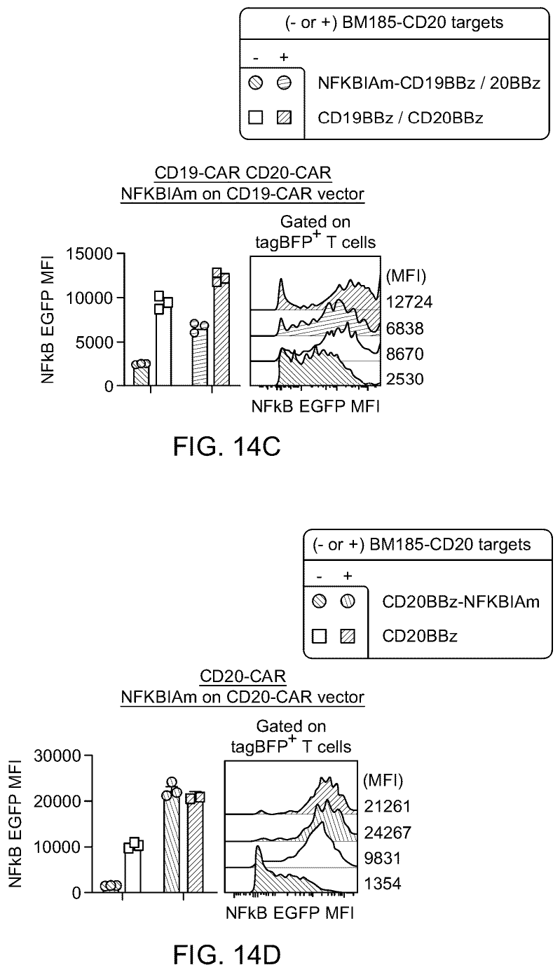

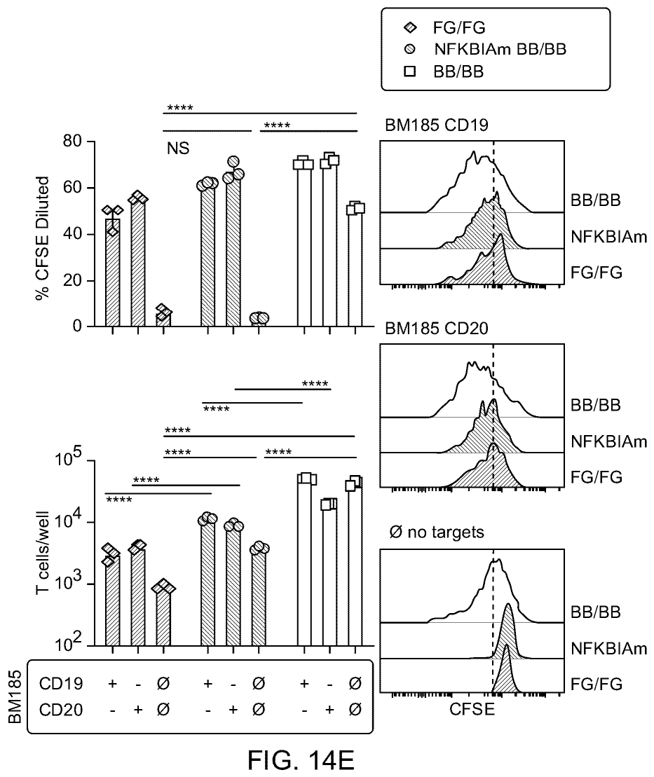

CD19/CD20 Dual CAR T cells were transduced with vectors-encoding HA-tagged NFKBIAm (FIG. 14A). FIG. 14B shows FACS analysis of HA-tagged NFKBIAm expression in the dual transduced CAR T cells. FIG. 14C shows FACS analysis of NFKB induction (EGFP) in zip-sorted CD19/CD20 dual-CAR T cells co-transduced with an SV40-BFP-NFκB-dEGFP reporter vector and stimulated for 24 h with BM185-CD20 targets (E:T=1:1). FIG. 14D shows NFκB reporter activity in T cells transduced with vectors encoding CD20BBz±NFBKIAm followed by co-culture with BM185-CD20 targets for 24 h (E:T=1:1). FIG. 14E shows target-stimulated spontaneous carboxyfluorescein diacetate, succinimidyl ester (CFSE) diluted and absolute T cell counts of dual-CAR T cells following 3 d culture with irradiated targets at (E:T=1:3). In the presence of antigen, NFKBIA inhibited T cells upregulate NFκB and divide but demonstrate reduced tonic activation resulting from high expression of two tonic signaling 4-1BB CARs. As a result, tonic proliferation in the absence of antigen is abrogated.

### 15.2.13 Reversal of Immune Phenotype Induced by Tonic Signaling of Vectors-Encoding NFkB Inhibitory Protein NFKBIAm for Inhibiting Tonic Signaling in Dual-CAR T Cells

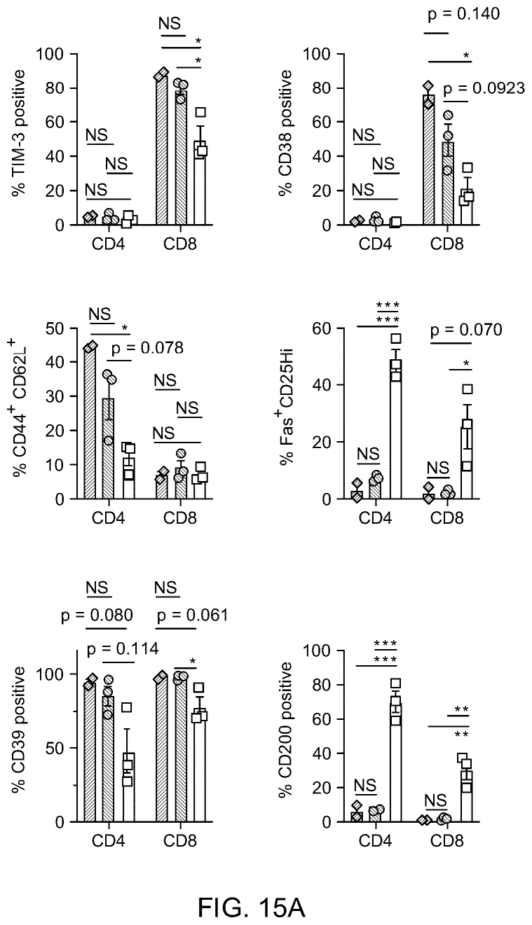

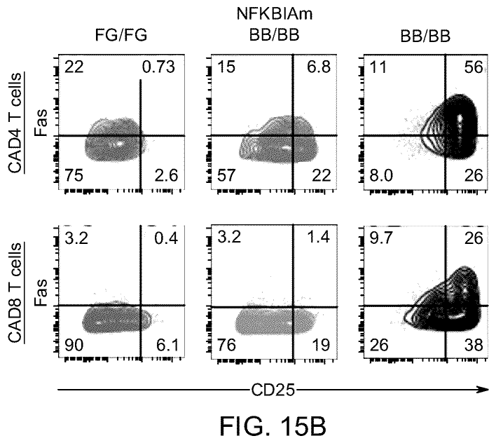

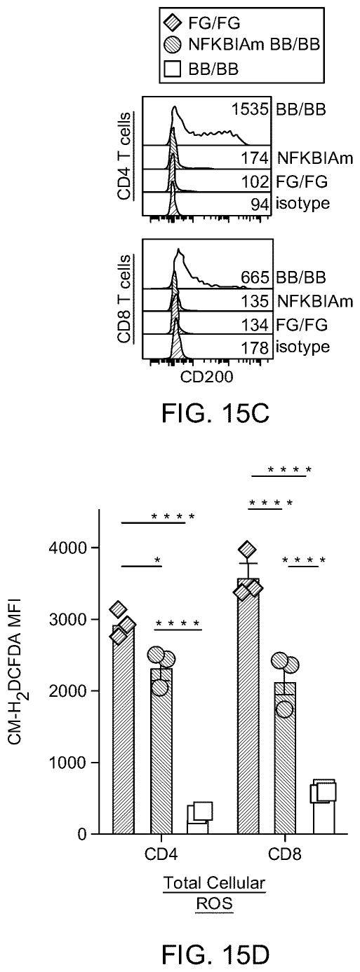

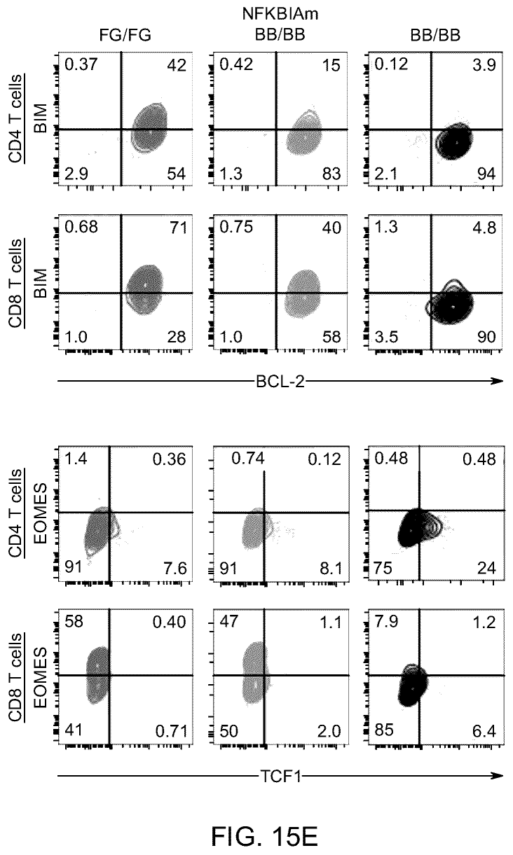

FIG. 15A shows expression of TIM-3, CD44/CD62L and CD39 in CD4+ and CD8+ dual-4-1BB CAR T cells. FIG. 15B shows FACS analysis of Fas and CD25 expression in unstimulated day 5 cultured Zip-sorted CD19/CD20 dual-CAR T cells (First generation (FG), NFKBIAm BB/BB, and BB/BB). FIG. 15C shows expression of CD200 in FG, NFKBIAm BB/BB, and BB/BB dual CAR T cells. FIG. 15D shows quantitation of total cellular ROS production in the T cells described in in FG, NFKBIAm BB/BB, and BB/BB dual CAR T cells following incubation with CM-H₂DCFDA. FIG. 15E shows the results of FACS analysis for transcription factors and apoptosis-associated proteins in T cells described in FG, NFKBIAm BB/BB, and BB/BB dual CAR T cells.

Conclusion: NFKBIAm-BB/BB and FG/FG T cells shared a similar immunophenotype, with upregulation of TIM-3, CD38, and CD39, and downregulation of Fas and CD25. NFKBIAm blocked tonic upregulation of the inhibitory ligand CD200 and upregulated total cellular ROS in BB/BB T cells. NFKBIAm expression partially upregulated the proapoptotic protein BIM which was repressed in BB/BB T cells. CD4+BB/BB T cells partially retained expression of the sternness factor TCF1, which was lost in FG/FG and NFKBIAm BB/BB T cells. Similarly, expression of the differentiation factor EOMES was repressed in CD8+BB/BB T cells but upregulated in FG/FG and NFKBIAm BB/BB T cells. Expression of NFKBIAm in BB/BB T cells blocked elevation of day 6 serum cytokines and rescued mice from early BB/BB CAR T cell-mediated toxicity, but also impaired anti-leukemia activity with mice demonstrating progressive BM185 growth and concomitant weight loss. Overall, we found that high expression of two 4-1BB co-stimulated CARs promotes tonic NFκB-dependent proliferation, and metabolic and immunophenotypic alterations, which transiently improved anti-leukemia activity, but also promoted lethal toxicity associated with cytokine release syndrome (CRS) and lethal toxicity. Taken together, these data show that immune phenotypes induced by tonic signaling in dual-4-1BB CAR T cells are reversed following NFκB inhibition.

### 15.2.14 Prevention of Toxicity and Cytokine Release In Vivo Following NFkB Inhibition

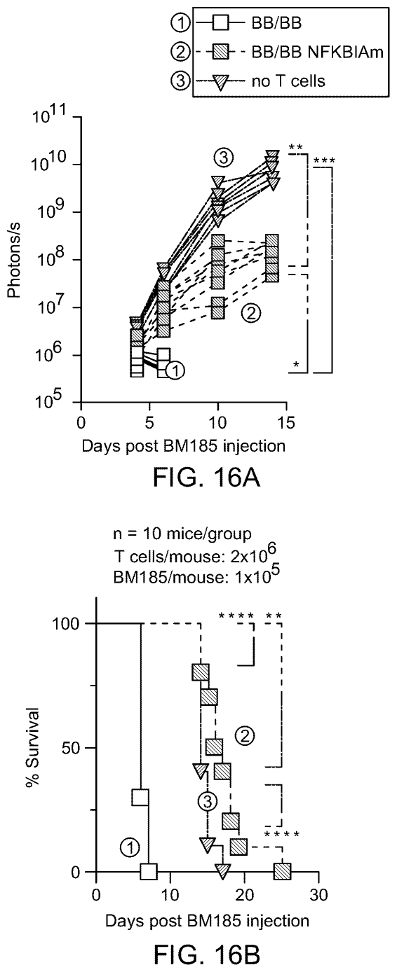

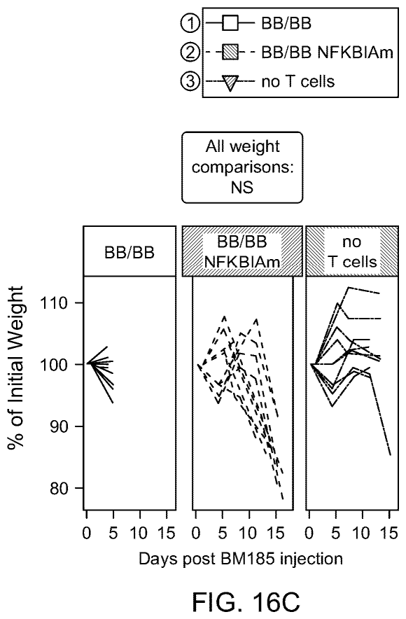

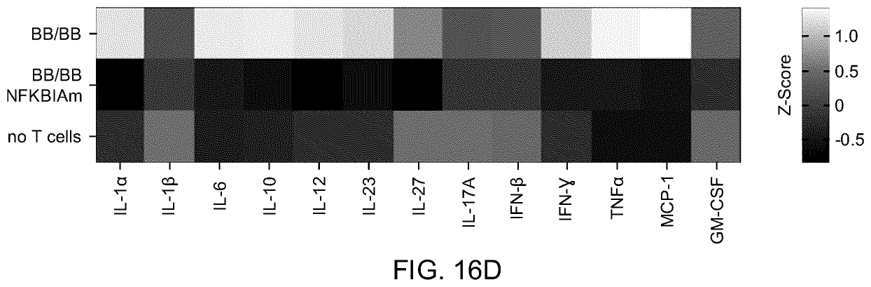

Dual-4-1BB-CAR T cell expressing NFκB inhibitors were tested for toxicity and cytokine release in vivo. BALB/c mice were injected with bioluminescent BM185 leukemic cells, followed by administration of zip sorted CD19BBz/CD20BBz dual CAR T cells or, CD19BBz/CD20BBz dual CAR T cells transduced with the NFKBIAm vector. FIG. 16A shows BM185 bioluminescence following administration of the dual CAR BALB/c T cells. Survival studies in these animals revealed improvement in survival in the NFKBIAm BB/BB group compared to the BB/BB group. FIG. 16C shows serial assessment of body weights post BM185 leukemia cell injection for the treated animals. FIG. 16D shows the results of serum cytokine analysis on day 6 post BM185 leukemia cell injection. In conclusion, dual-4-1BB CAR T cells promote lethal toxicity shortly after infusion associated with upregulation of multiple inflammatory cytokines in the serum of mice. Co-expression of NFKBIAm inhibited T cell-mediated toxicity and cytokine production with retention of anti-leukemia activity.

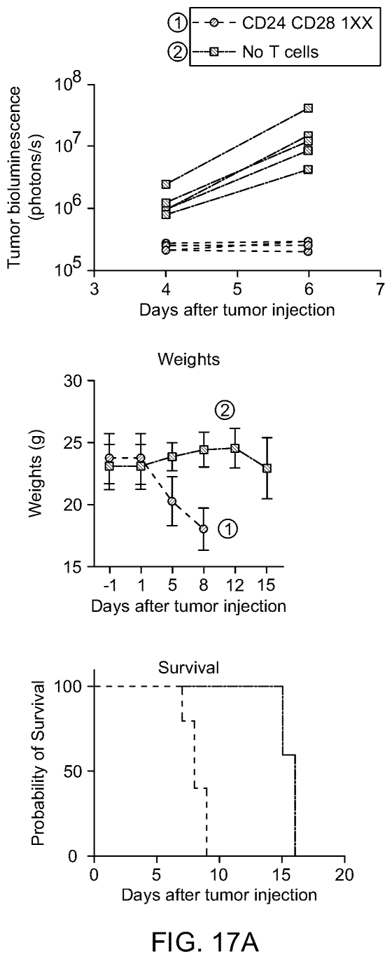

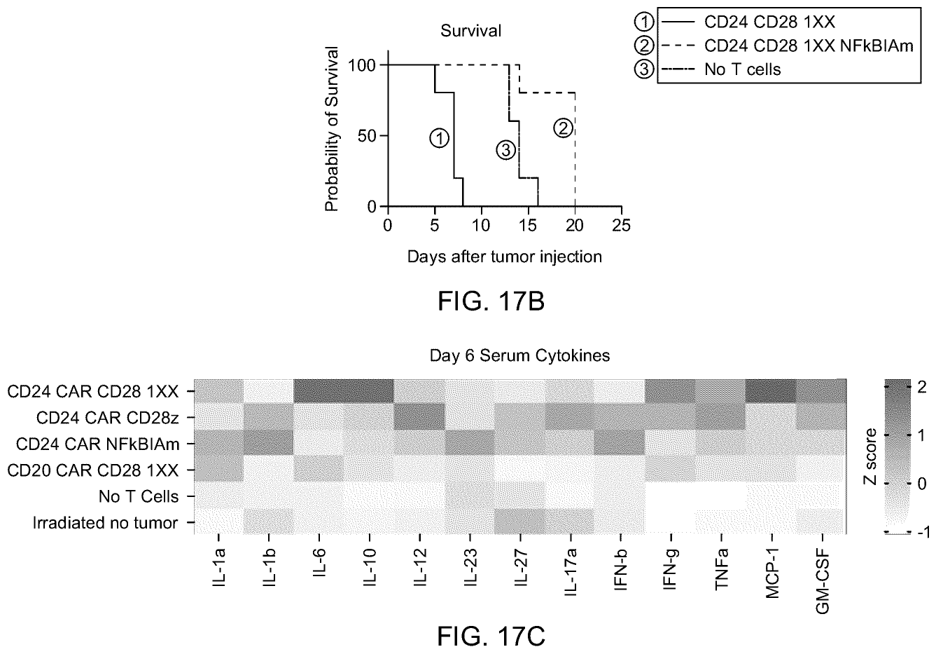

A similar analysis was performed to test the effects of NFκB inhibition in CD24-CAR T cells FIG. 17A shows tumor fluorescence, body weights and survival of BALB/c mice injected with BM185 leukemic cells followed by administration with CD24-CAR T cells. FIG. 17B shows survival of BALB/c mice injected with BM185 leukemic cells followed by administration with CD24-CAR T cells or CD24-CAR T cells transduced with the NFKBIAm vector. FIG. 17C shows the results of serum cytokine analysis on day 6 post BM185 leukemia cell injection in mice administered the indicated CAR T cells. In conclusion, co-expression of the NFKBIAm inhibited T cell-mediated toxicity and cytokine production in the CAR T cells.

### 15.2.15 NFAT-Inducible NFKBIAm Expression

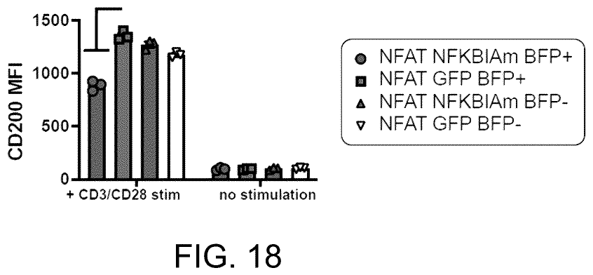

T cells were transduced with NFAT-inducible NFKBIAm vector (see FIGS. 5A-5D) and stimulated for 24 h with plate-bound CD3/CD28 (2 μg/mL each, 24 h). FIG. 18 shows that CD200 upregulation was inhibited in cells expressing the negative feedback NFAT-NFKBIAm-EGFP vector (BFP⁺), but not in non-transduced cells (BFP⁻) or control NFAT-EGFP vector.

### 15.2.16 Drug-Inducible NFKBIAm Expression

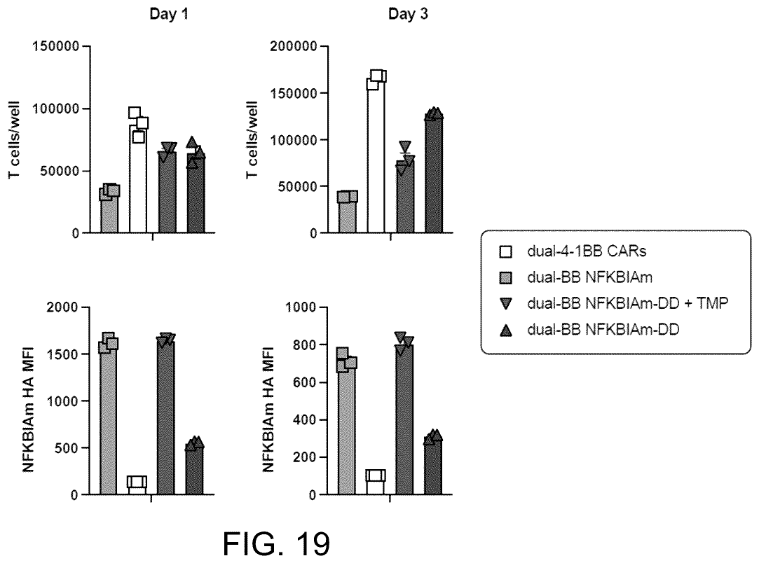

Zip-sorted CD19BBz/CD20BBz dual-CAR T cells were transduced with the NFKBIAm vector, or the DHFR-DD-NFKBIAm vector and cultured with TMP for 1 or 3 days. T cell counts and NFKBIAm expression (HA-tag) were assessed (FIG. 19). Tonic T cell proliferation was attenuated in the presence of constitutive NFKBIAm expression and drug induced NFKBIAm expression.

Overall, the results presented herein demonstrate that the instant platform facilitates generation of T cells expressing combinations of many transgenes at high expression levels able to simultaneously overcome multiple challenges faced by CAR T cells. This platform showed remarkable versatility, allowing us to generate capture-zippers from a wide array of molecules. Tonic signaling from CD28-costimulated CARs promoted upregulation of inhibitory receptors, exhaustion-associated upregulation of TOX and downregulation of TCF1 transcription factors, and increased production of ROS. Partial ITAM inactivation and selective NFAT attenuation greatly improved dual-CAR T cell activity suggesting that WT CD28z CARs promote T cell dysfunction in part through NFAT-mediated upregulation of exhaustion-associated factors including TOX, with NFAT signaling potentially enhanced by CAR-induced ROS elevation. In contrast, expression of two 4-1BB-costimulated CARs produced autonomously expanding T cells characterized by down-regulation of ROS and a subset of inhibitory receptors and associated with lethal CRS, which could be prevented via canonical NFκB pathway inhibition. Therefore, depending on the pathways activated by tonic signaling, high-level multi-CAR expression can drive either T cell exhaustion or promote T cell-mediated toxicity.

1. A system comprising a plurality of nucleic acid constructs, wherein the plurality comprises:
(a) a first nucleic acid construct encoding a membrane bound polypeptide comprising:
(i) an extracellular domain comprising a first leucine zipper sequence;
(ii) a transmembrane domain; and
(iii) an intracellular domain;
(b) a second nucleic acid construct encoding a soluble polypeptide comprising a second leucine zipper sequence capable of heterodimerizing with the first leucine zipper sequence and a signal peptide sequence;
wherein each of the first and/or second nucleic acid constructs further encode one or more CAR, safety switch, switch receptor, and/or cytokine; and
wherein the first or second nucleic acid construct further comprises a nucleic acid sequence encoding: (1) a nuclear factor of activated T cells (NFAT) inhibitor peptide or NFkB inhibitor peptide; and (2) a carrier protein.

2. The system of claim 1, wherein the inhibitor peptide inhibits calcineurin signaling.

3. The system of claim 1, wherein the inhibitor peptide is a NFAT inhibitor peptide (NFAT inhibitor).

4. The system of claim 3, wherein:
a) the NFAT inhibitor comprises a PXIXIT calcineurin binding sequence, wherein X is any amino acid;
b) the NFAT inhibitor is a VIVIT peptide or a variant thereof; or
c) the NFAT inhibitor comprises the amino acid sequence set forth in any one of SEQ ID Nos: 24-29.

5.-6. (canceled)

7. The system of claim 2, wherein the inhibitor peptide comprises a sequence derived from the autoinhibitory domain of calcineurin.

8. The system of claim 7, wherein the inhibitor peptide comprises the amino acid sequence set forth in SEQ ID No: 30.

9. The system of claim 1, wherein the inhibitor peptide inhibits NFkB signaling.

10. The system of claim 9, wherein the inhibitor peptide is an NFkB inhibitor, or a degradation-resistant mutant thereof.

11. The system of claim 10, wherein the inhibitory peptide is a mutated NFkB inhibitor alpha (NFKBIAm).

12. The system of claim 11, wherein the NFKBIAm comprises the amino acid sequence set forth in SEQ ID No: 32.

13. The system of claim 1, wherein the carrier protein is selected from the group consisting of glutathione-S-transferase (GSTA1), enhanced green fluorescence protein (EGFP), and blue fluorescent (BFP).

14. The system of claim 1, wherein the nucleic acid sequence encoding the CP comprises a regulatable gene element.

15.-21. (canceled)

22. The system of claim 1, wherein the first nucleic acid construct comprises the amino acid sequence set forth in SEQ ID NO: 34.

23. The system of claim 1, wherein the second nucleic acid construct comprises the amino acid sequence set forth in SEQ ID NO: 35.

24. The system of claim 13, wherein the nucleic acid sequence of the first nucleic acid construct further encodes a hinge domain.

25. (canceled)

26. The system of claim 1, wherein the first and/or second nucleic acid construct comprises a promoter element that regulates expression of the inhibitor peptide.

27.-29. (canceled)

30. The system of claim 1, wherein the inhibitory peptide comprises the amino acid sequence set forth in any one of SEQ ID NOs: 70-93.

31. An engineered immune cell comprising the system of claim 1.

32.-64. (canceled)

65. A method for treating a disease comprising providing to a subject in need thereof a population of engineered immune cells wherein the engineered immune cells each comprise the system of claim 1.

66. (canceled)

67. The method of claim 65, wherein the disease is a cancer, an autoimmune disease, an inflammatory disease, or a graft versus-host disease.

68.-73. (canceled)


SEQUENCE LISTING (93 sequences)

SEQ ID NO: 1 - protein, 6 aa, synthetic construct
  REGION 1..6: note = Description of sequence: Linker1
GSGRSG

SEQ ID NO: 2 - protein, 9 aa, synthetic construct
  REGION 1..9: note = Description of sequence: Linker2
GGGGSGGGS

SEQ ID NO: 3 - protein, 17 aa, synthetic construct
  REGION 1..17: note = Description of sequence: Linker3
GGGGSGGGGSGGGGSTS

SEQ ID NO: 4 - protein, 12 aa, synthetic construct
  REGION 1..12: note = Description of sequence: Linker4
GPPVASGSGRSG

SEQ ID NO: 5 - protein, 12 aa, synthetic construct
  REGION 1..12: note = Description of sequence: Linker5
GGGGSGGGSGSL

SEQ ID NO: 6 - protein, 37 aa, synthetic construct
  REGION 1..37: note = Description of sequence: Linker6
GGGGSGGGSELPTQGTFSNVSTNVSGGGGSGGGSRSG

SEQ ID NO: 7 - protein, 12 aa, synthetic construct
  REGION 1..12: note = Description of sequence: Linker7
TSGGGGSGGGSG

SEQ ID NO: 8 - protein, 14 aa, synthetic construct
  REGION 1..14: note = Description of sequence: Linker8
TSGGGGSGGGSGTG

SEQ ID NO: 9 - protein, 12 aa, synthetic construct
  REGION 1..12: note = Description of sequence: Linker9
GGGGSGGGSGTG

SEQ ID NO: 10 - protein, 11 aa, synthetic construct
  REGION 1..11: note = Description of sequence: Linker10
STGGGGSGGGS

SEQ ID NO: 11 - protein, 11 aa, synthetic construct
  REGION 1..11: note = Description of sequence: Linker11
GSGGGGSGGGS

SEQ ID NO: 12 - protein, 10 aa, synthetic construct
  REGION 1..10: note = Description of sequence: Linker12
GTSRAKRGSG

SEQ ID NO: 13 - protein, 8 aa, synthetic construct
  REGION 1..8: note = Description of sequence: Linker13
GGGGSGTG

SEQ ID NO: 14 - protein, 9 aa, synthetic construct
  REGION 1..9: note = Description of sequence: Linker14
GTSRAKRGS

SEQ ID NO: 15 - protein, 9 aa, synthetic construct
  REGION 1..9: note = Description of sequence: Linker15
ALGGSGGGS

SEQ ID NO: 16 - protein, 7 aa, synthetic construct
  REGION 1..7: note = Description of sequence: Linker16
GGGGSTS

SEQ ID NO: 17 - protein, 7 aa, synthetic construct
  REGION 1..7: note = Description of sequence: Linker17
SGGGGSD

SEQ ID NO: 18 - protein, 6 aa, synthetic construct
  REGION 1..6: note = Description of sequence: Linker18
GSSGSG

SEQ ID NO: 19 - protein, 6 aa, synthetic construct
  REGION 1..6: note = Description of sequence: Linker19
GSGGTR

SEQ ID NO: 20 - protein, 5 aa, synthetic construct
  REGION 1..5: note = Description of sequence: Linker20
GSGTR

SEQ ID NO: 21 - protein, 5 aa, synthetic construct
  REGION 1..5: note = Description of sequence: Linker21
TSGSG

SEQ ID NO: 22 - protein, 5 aa, synthetic construct
  REGION 1..5: note = Description of sequence: Linker22
GSGGS

SEQ ID NO: 23 - protein, 18 aa, synthetic construct
  REGION 1..18: note = Description of sequence: interchain linker
GSTGGGGSGGGGSGGGGS

SEQ ID NO: 24 - protein, 22 aa, synthetic construct
  REGION 1..22: note = Description of sequence: VIVIT peptide
MAGPHPVIVITGPHEEGPPVAS

SEQ ID NO: 25 - protein, 22 aa, synthetic construct
  REGION 1..22: note = Description of sequence: Vmut1
MAGPVPRIIITKPYEEGPPVAS

SEQ ID NO: 26 - protein, 22 aa, synthetic construct
  REGION 1..22: note = Description of sequence: Vmut2
MAGPVPRIIITKPDEEGPPVAS

SEQ ID NO: 27 - protein, 22 aa, synthetic construct
  REGION 1..22: note = Description of sequence: Vmut3
MAGPVPRIIITKPEEEGPPVAS

SEQ ID NO: 28 - protein, 84 aa, synthetic construct
  REGION 1..84: note = Description of sequence: Vmut3 3x
MAGPVPRIIITKPEEEGSTGGGGSGGGGSGGGGSMAGPVPRIIITKPEEEGSTGGGGSGGGGSGGGGSMAGPVPRIIITKPEEE

SEQ ID NO: 29 - protein, 23 aa, synthetic construct
  REGION 1..23: note = Description of sequence: Vmut5
MASGLLPRIIITPPYELGPPVAS

SEQ ID NO: 30 - protein, 25 aa, synthetic construct
  REGION 1..25: note = Description of sequence: DAMP
ITSFEEAKGLDRINERMPPRRDAMP

SEQ ID NO: 31 - protein, 37 aa, synthetic construct
  REGION 1..37: note = Description of sequence: linker + DAMP peptide
  DOMAIN 1..12: note = Linker Domain
  DOMAIN 13..37: note = DAMP Peptide
GGGGSGGGSGSLITSFEEAKGLDRINERMPPRRDAMP

SEQ ID NO: 32 - protein, 314 aa, synthetic construct
  REGION 1..314: note = Description of sequence: NFKBIAm (mutant IkBa)
MFQPAGHGQDWAMEGPRDGLKKERLVDDRHDAGLDAMKDEEYEQMVKELREIRLQPQEAPLAAEPWKQQLTEDGDSFLHLAIIHEEKPLTMEVIGQVKGDLAFLNFQNNLQQTPLHLAVITNQPGIAEALLKAGCDPELRDFRGNTPLHLACEQGCLASVAVLTQTCTPQHLHSVLQATNYNGHTCLHLASIHGYLAIVEHLVTLGADVNAQEPCNGRTALHLAVDLQNPDLVSLLLKCGADVNRVTYQGYSPYQLTWGRPSTRIQQQLGQLTLENLQMLPEAEDEEAYDAEAEFAEDELPDDDCVFGGQRLTL

SEQ ID NO: 33 - protein, 527 aa, synthetic construct
  REGION 1..527: note = Description of sequence: NFKBIAm DHFR-DD
YPYDVPDYAGYPYDVPDYAGYPYDVPDYAGSGMFQPAGHGQDWAMEGPRDGLKKERLVDDRHDAGLDAMKDEEYEQMVKELREIRLQPQEAPLAAEPWKQQLTEDGDSFLHLAIIHEEKPLTMEVIGQVKGDLAFLNFQNNLQQTPLHLAVITNQPGIAEALLKAGCDPELRDFRGNTPLHLACEQGCLASVAVLTQTCTPQHLHSVLQATNYNGHTCLHLASIHGYLAIVEHLVTLGADVNAQEPCNGRTALHLAVDLQNPDLVSLLLKCGADVNRVTYQGYSPYQLTWGRPSTRIQQQLGQLTLENLQMLPEAEDEEAYDAEAEFAEDELPDDDCVFGGQRLTLGSGTRGSTGGGGSGGGGSGGGGSISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 34 - protein, 47 aa, synthetic construct
  REGION 1..47: note = Description of sequence: EE12RR345L zipper
LEIEAAFLERENTALETRVAELRQRVQRLRNRVSQYRTRYGPLGGGK

SEQ ID NO: 35 - protein, 47 aa, synthetic construct
  REGION 1..47: note = Description of sequence: RR12EE345L zipper
LEIRAAFLRQRNTALRTEVAELEQEVQRLENEVSQYETRYGPLGGGK

SEQ ID NO: 36 - protein, 113 aa, synthetic construct
  REGION 1..113: note = Description of sequence: 3N RR EE blocked zipper
LEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGK

SEQ ID NO: 37 - protein, 113 aa, synthetic construct
  REGION 1..113: note = Description of sequence: RR EE blocked zipper
LEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGK

SEQ ID NO: 38 - protein, 47 aa, synthetic construct
  REGION 1..47: note = Description of sequence: 3N mutant zipper
LEIEAAFLRQENTALRTRVAELEQEVQRLENEVSQYETRYGPLGGGK

SEQ ID NO: 39 - protein, 29 aa, synthetic construct
  REGION 1..29: note = Description of sequence: 3x HA tag
YPYDVPDYAGYPYDVPDYAGYPYDVPDYA

SEQ ID NO: 40 - protein, 39 aa, synthetic construct
  REGION 1..39: note = Description of sequence: twin Strep Tag
SAWSHPQFEKGGGSGGGSGGSAWSHPQFEKGGGGSGGGS

SEQ ID NO: 41 - protein, 233 aa, synthetic construct
  REGION 1..233: note = Description of sequence: tagBFP
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 42 - protein, 239 aa, synthetic construct
  REGION 1..239: note = Description of sequence: EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 43 - protein, 159 aa, synthetic construct
  REGION 1..159: note = Description of sequence: DHFR-DD
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 44 - protein, 20 aa, synthetic construct
  REGION 1..20: note = Description of sequence: E2A peptide
QCTNYALLKLAGDVESNPGP

SEQ ID NO: 45 - protein, 235 aa, synthetic construct
  REGION 1..235: note = Description of sequence: human CD8 alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 46 - protein, 222 aa, synthetic construct
  REGION 1..222: note = Description of sequence: glutathione-S-transferase (GSTA1)
MAEKPKLHYFNARGRMESTRWLLAAAGVEFEEKFIKSAEDLDKLRNDGYLMFQQVPMVEIDGMKLVQTRAILNYIASKYNLYGKDIKERALIDMYIEGIADLGEMILLLPVCPPEEKDAKLALIKEKIKNRYFPAFEKVLKSHGQDYLVGNKLSRADIHLVELLYYVEELDSSLISSFPLLKALKTRISNLPTVKKFLQPGSPRKPPMDEKSLEEARKIFRF

SEQ ID NO: 47 - protein, 402 aa, synthetic construct
  REGION 1..402: note = Description of sequence: SFFV promoter
GTAACGCCATTTTGCAAGGCATGGAAAAATACCAAACCAAGAATAGAGAAGTTCAGATCAAGGGCGGGTACATGAAAATAGCTAACGTTGGGCCAAACAGGATATCTGCGGTGAGCAGTTTCGGCCCCGGCCCGGGGCCAAGAACAGATGGTCACCGCAGTTTCGGCCCCGGCCCGAGGCCAAGAACAGATGGTCCCCAGATATGGCCCAACCCTCAGCAGTTTCTTAAGACCCATCAGATGTTTCCAGGCTCCCCCAAGGACCTGAAATGACCCTGCGCCTTATTTGAATTAACCAATCAGCCTGCTTCTCGCTTCTGTTCGCGCGCTTCTGCTTCCCGAGCTCTATAAAAGAGCTCACAACCCCTCACTCGGCGCGCCAGTCCTCCGACAGACTGAGTCG

SEQ ID NO: 48 - protein, 352 aa, synthetic construct
  REGION 1..352: note = Description of sequence: NFAT response element
GGAGGAAAAACTGTTTCATACAGAAGGCGTCAATTAGGAGGAAAAACTGTTTCATACAGAAGGCGTCAATTAGGAGGAAAAACTGTTTCATACAGAAGGCGTCAATTGGTCCCATCGAATTAGGAGGAAAAACTGTTTCATACAGAAGGCGTCAATTAGGAGGAAAAACTGTTTCATACAGAAGGCGTCAATTAGGAGGAAAAACTGTTTCATACAGAAGGCGTCAATTGGTCCCGGGACATTTTGACACCCCCATAATATTTTTCCAGAATTAACAGTATAAATTGCATCTCTTGTTCAAGAGTTCCCTATCACTCTCTTTAATCACTACTCACAGTAACCTCAACTCCTG

SEQ ID NO: 49 - protein, 110 aa, synthetic construct
  REGION 1..110: note = Description of sequence: NFKB response element
GGGGACTTTCCGCTTGGGGACTTTCCGCTGGGGACTTTCCGCTGGGGACTTTCCGCTGGGGACTTTCCGCGGAGACTCTAGAGGGTATATAATGGAAGCTCGAATTCCAG

SEQ ID NO: 50 - protein, 49 aa, synthetic construct
  REGION 1..49: note = Description of sequence: poly(A) signal
AATAAAATATCTTTATTTTCATTACATCTGTGTGTTGGTTTTTTGTGTG

SEQ ID NO: 51 - protein, 40 aa, synthetic construct
  REGION 1..40: note = Description of sequence: R2 - dual hCD20 cyclic mimotope with intervening linkers
CPYSNPSLCSGGGGSSGGGGSDCPYSNPSLCGGGGSGGGS

SEQ ID NO: 52 - protein, 25 aa, synthetic construct
  REGION 1..25: note = Description of sequence: PD-1 hinge
TERILETSTRYPSPSPKPEGRFQGM

SEQ ID NO: 53 - protein, 27 aa, synthetic construct
  REGION 1..27: note = Description of sequence: CD28TM
FWALVVVAGVLFCYGLLVTVALCVIWT

SEQ ID NO: 54 - protein, 36 aa, synthetic construct
  REGION 1..36: note = Description of sequence: CD28 hinge
IEFMYPPPYLDNERSNGTIIHIKEKHLCHTQSSPKL

SEQ ID NO: 55 - protein, 41 aa, synthetic construct
  REGION 1..41: note = Description of sequence: CD28 cytoplasmic
NSRRNRLLQSDYMNMTPRRPGLTRKPYQPYAPARDFAAYRP

SEQ ID NO: 56 - protein, 42 aa, synthetic construct
  REGION 1..42: note = Description of sequence: human 4-1BB cytoplasmic domain
KRGRKKLLYIFKQPFMRPVQTTQEEDGCSCRFPEEEEGGCEL

SEQ ID NO: 57 - protein, 29 aa, synthetic construct
  REGION 1..29: note = Description of sequence: CD3zeta delta
RAKFSRSAETAANLQDTFDALHMQTLAPR

SEQ ID NO: 58 - protein, 113 aa, synthetic construct
  REGION 1..113: note = Description of sequence: CD3zeta
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 59 - protein, 19 aa, synthetic construct
  REGION 1..19: note = Description of sequence: P2A
ATNFSLLKQAGDVEENPGP

SEQ ID NO: 60 - protein, 41 aa, synthetic construct
  REGION 1..41: note = Description of sequence: Q2 CD34 zipper
ELPTQGTFSNVSTNVSGGGGSGGGSELPTQGTFSNVSTNVS

SEQ ID NO: 61 - protein, 400 aa, synthetic construct
  REGION 1..400: note = Description of sequence: iCaspase9
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVD

SEQ ID NO: 62 - protein, 44 aa, synthetic construct
  REGION 1..44: note = Description of sequence: CD8 hinge
STTTKPVLRTPSPVHPTGTSQPQRPEDCRPRGSVKGTGLDFACD

SEQ ID NO: 63 - protein, 25 aa, synthetic construct
  REGION 1..25: note = Description of sequence: CD8TM
IYIWAPLAGICVALLLSLIITLICY

SEQ ID NO: 64 - protein, 22 aa, synthetic construct
  REGION 1..22: note = Description of sequence: F2A
VKQTLNFDLLKLAGDVESNPGP

SEQ ID NO: 65 - protein, 55 aa, synthetic construct
  REGION 1..55: note = Description of sequence: FLAG RR zipper
DYKDDDDKLEIRAAFLRQRNTALRTEVAELEQEVQRLENEVSQYETRYGPLGGGK

SEQ ID NO: 66 - protein, 143 aa, synthetic construct
  REGION 1..143: note = Description of sequence: Thy1.1
QKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 67 - protein, 240 aa, synthetic construct
  REGION 1..240: note = Description of sequence: clone M1/69 CD24 scFv
EVKLQESGPGLVKPSLTLSLTCTVSGFSLNNYAVIWVRQPPGKGLEWMGVIWSDGKTNYNSALKSRLSISRDTSKSQVFLKMNNLQTEDTAMYFCARSSYYSSWFDSWGQGVMVTVSSGGGGSGGGGSGGGGSDIQLTQSPSFLSASVGDRVTLSCKASQNINKYLAWYQQKLGEAPKLLIYNANSLQTGIPSRFSGSGSGTDFTLTISSLQPEDVATYFCLQHNSWWTLGGGTKLELKR

SEQ ID NO: 68 - protein, 239 aa, synthetic construct
  REGION 1..239: note = Description of sequence: 1D3 CD19 scFv - VH-(G4S)3-VL (CD19 scFv)
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKR

SEQ ID NO: 69 - protein, 271 aa, synthetic construct
  REGION 1..271: note = Description of sequence: 18B12 scFv mCD20 targeting - Patent: WO2007064911A1
TMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSS

SEQ ID NO: 70 - protein, 267 aa, synthetic construct
  REGION 1..267: note = Description of sequence: VIVIT-EGFP
MAGPHPVIVITGPHEEGPPVASGSGRSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 71 - protein, 479 aa, synthetic construct
  REGION 1..479: note = Description of sequence: VIVIT 3xHA DHFR-DD N type hCD8
MAGPHPVIVITGPHEEGPPVASGSGRSGYPYDVPDYAGYPYDVPDYAGYPYDVPDYAGSGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGQCTNYALLKLAGDVESNPGPLEMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 72 - protein, 479 aa, synthetic construct
  REGION 1..479: note = Description of sequence: Vmut3 3xHA DHFR-DD N type hCD8
MAGPVPRIIITKPEEEGPPVASGSGRSGYPYDVPDYAGYPYDVPDYAGYPYDVPDYAGSGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGQCTNYALLKLAGDVESNPGPLEMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 73 - protein, 542 aa, synthetic construct
  REGION 1..542: note = Description of sequence: VIVIT GSTA1 3xHA hCD8
MAGPHPVIVITGPHEEGPPVASGSGRSGMAEKPKLHYFNARGRMESTRWLLAAAGVEFEEKFIKSAEDLDKLRNDGYLMFQQVPMVEIDGMKLVQTRAILNYIASKYNLYGKDIKERALIDMYIEGIADLGEMILLLPVCPPEEKDAKLALIKEKIKNRYFPAFEKVLKSHGQDYLVGNKLSRADIHLVELLYYVEELDSSLISSFPLLKALKTRISNLPTVKKFLQPGSPRKPPMDEKSLEEARKIFRFGTGYPYDVPDYAGYPYDVPDYAGYPYDVPDYAALGQCTNYALLKLAGDVESNPGPLEMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 74 - protein, 542 aa, synthetic construct
  REGION 1..542: note = Description of sequence: Vmut3 GSTA1 3xHA hCD8
MAGPVPRIIITKPEEEGPPVASGSGRSGMAEKPKLHYFNARGRMESTRWLLAAAGVEFEEKFIKSAEDLDKLRNDGYLMFQQVPMVEIDGMKLVQTRAILNYIASKYNLYGKDIKERALIDMYIEGIADLGEMILLLPVCPPEEKDAKLALIKEKIKNRYFPAFEKVLKSHGQDYLVGNKLSRADIHLVELLYYVEELDSSLISSFPLLKALKTRISNLPTVKKFLQPGSPRKPPMDEKSLEEARKIFRFGTGYPYDVPDYAGYPYDVPDYAGYPYDVPDYAALGQCTNYALLKLAGDVESNPGPLEMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 75 - protein, 267 aa, synthetic construct
  REGION 1..267: note = Description of sequence: VIVIT GFP - Vmut1
MAGPVPRIIITKPYEEGPPVASGSGRSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 76 - protein, 267 aa, synthetic construct
  REGION 1..267: note = Description of sequence: VIVIT GFP - Vmut2
MAGPVPRIIITKPDEEGPPVASGSGRSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 77 - protein, 267 aa, synthetic construct
  REGION 1..267: note = Description of sequence: VIVIT GFP - Vmut3
MAGPVPRIIITKPEEEGPPVASGSGRSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - protein, 268 aa, synthetic construct
  REGION 1..268: note = Description of sequence: VIVIT GFP - Vmut5
MASGLLPRIIITPPYELGPPVASGSGRSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 79 - protein, 2371 aa, synthetic construct
  REGION 1..2371: note = Description of sequence: VIVIT GFP NFAT SFFV BFP
CACACAAAAAACCAACACACAGATGTAATGAAAATAAAGATATTTTATTTAAGCTTTACTTGTACAGCTCGTCCATGCCGAGAGTGATCCCGGCGGCGGTCACGAACTCCAGCAGGACCATGTGATCGCGCTTCTCGTTGGGGTCTTTGCTCAGGGCGGACTGGGTGCTCAGGTAGTGGTTGTCGGGCAGCAGCACGGGGCCGTCGCCGATGGGGGTGTTCTGCTGGTAGTGGTCGGCGAGCTGCACGCTGCCGTCCTCGATGTTGTGGCGGATCTTGAAGTTCACCTTGATGCCGTTCTTCTGCTTGTCGGCCATGATATAGACGTTGTGGCTGTTGTAGTTGTACTCCAGCTTGTGCCCCAGGATGTTGCCGTCCTCCTTGAAGTCGATGCCCTTCAGCTCGATGCGGTTCACCAGGGTGTCGCCCTCGAACTTCACCTCGGCGCGGGTCTTGTAGTTGCCGTCGTCCTTGAAGAAGATGGTGCGCTCCTGGACGTAGCCTTCGGGCATGGCGGACTTGAAGAAGTCGTGCTGCTTCATGTGGTCGGGGTAGCGGCTGAAGCACTGCACGCCGTAGGTCAGGGTGGTCACGAGGGTGGGCCAGGGCACGGGCAGCTTGCCGGTGGTGCAGATGAACTTCAGGGTCAGCTTGCCGTAGGTGGCATCGCCCTCGCCCTCGCCGGACACGCTGAACTTGTGGCCGTTTACGTCGCCGTCCAGCTCGACCAGGATGGGCACCACCCCGGTGAACAGCTCCTCGCCCTTGCTCACCATCCCAGATCTTCCTGATCCGCTTGCGACAGGGGGCCCCTCCTCGTGTGGACCTGTAATTACGATTACTGGGTGTGGTCCCGCCATGGTACGCGTCAGGAGTTGAGGTTACTGTGAGTAGTGATTAAAGAGAGTGATAGGGAACTCTTGAACAAGAGATGCAATTTATACTGTTAATTCTGGAAAAATATTATGGGGGTGTCAAAATGTCCCGGGACCAATTGACGCCTTCTGTATGAAACAGTTTTTCCTCCTAATTGACGCCTTCTGTATGAAACAGTTTTTCCTCCTAATTGACGCCTTCTGTATGAAACAGTTTTTCCTCCTAATTCGATGGGACCAATTGACGCCTTCTGTATGAAACAGTTTTTCCTCCTAATTGACGCCTTCTGTATGAAACAGTTTTTCCTCCTAATTGACGCCTTCTGTATGAAACAGTTTTTCCTCCTAATTCGATATCAAGCTGGCGGCCGCAACTGGCCGGTACCTGTAACGCCATTTTGCAAGGCATGGAAAAATACCAAACCAAGAATAGAGAAGTTCAGATCAAGGGCGGGTACATGAAAATAGCTAACGTTGGGCCAAACAGGATATCTGCGGTGAGCAGTTTCGGCCCCGGCCCGGGGCCAAGAACAGATGGTCACCGCAGTTTCGGCCCCGGCCCGAGGCCAAGAACAGATGGTCCCCAGATATGGCCCAACCCTCAGCAGTTTCTTAAGACCCATCAGATGTTTCCAGGCTCCCCCAAGGACCTGAAATGACCCTGCGCCTTATTTGAATTAACCAATCAGCCTGCTTCTCGCTTCTGTTCGCGCGCTTCTGCTTCCCGAGCTCTATAAAAGAGCTCACAACCCCTCACTCGGCGCGCCAGTCCTCCGACAGACTGAGTCGGGATCCACCATGAGCGAGCTGATTAAGGAGAACATGCACATGAAGCTGTACATGGAGGGCACCGTGGACAACCATCACTTCAAGTGCACATCCGAGGGCGAAGGCAAGCCCTACGAGGGCACCCAGACCATGAGAATCAAGGTGGTCGAGGGCGGCCCTCTCCCCTTCGCCTTCGACATCCTGGCTACTAGCTTCCTCTACGGCAGCAAGACCTTCATCAACCACACCCAGGGCATCCCCGACTTTTTCAAGCAGTCCTTCCCTGAGGGCTTCACATGGGAGAGAGTCACCACATACGAAGACGGGGGCGTGCTGACCGCTACCCAGGACACCAGCCTCCAGGACGGCTGCCTCATCTACAACGTCAAGATCAGAGGGGTGAACTTTACATCCAACGGCCCTGTGATGCAGAAGAAAACACTCGGCTGGGAGGCCTTCACCGAGACGCTGTACCCCGCTGACGGCGGCCTGGAAGGCAGAAACGACATGGCCCTGAAGCTCGTGGGCGGGAGCCATCTGATCGCAAACATCAAGACCACATATAGATCCAAGAAACCCGCTAAGAACCTCAAGATGCCTGGCGTCTACTATGTGGACTACAGACTGGAAAGAATCAAGGAGGCCAACAACGAGACCTACGTCGAGCAGCACGAGGTGGCAGTGGCCAGATACTGCGACCTCCCTAGCAAACTGGGCCACAAGCTAAAT

SEQ ID NO: 80 - protein, 2155 aa, synthetic construct
  REGION 1..2155: note = Description of sequence: VIVIT GFP NFkB SFFV BFP
CACACAAAAAACCAACACACAGATGTAATGAAAATAAAGATATTTTATTTAAGCTTTACTTGTACAGCTCGTCCATGCCGAGAGTGATCCCGGCGGCGGTCACGAACTCCAGCAGGACCATGTGATCGCGCTTCTCGTTGGGGTCTTTGCTCAGGGCGGACTGGGTGCTCAGGTAGTGGTTGTCGGGCAGCAGCACGGGGCCGTCGCCGATGGGGGTGTTCTGCTGGTAGTGGTCGGCGAGCTGCACGCTGCCGTCCTCGATGTTGTGGCGGATCTTGAAGTTCACCTTGATGCCGTTCTTCTGCTTGTCGGCCATGATATAGACGTTGTGGCTGTTGTAGTTGTACTCCAGCTTGTGCCCCAGGATGTTGCCGTCCTCCTTGAAGTCGATGCCCTTCAGCTCGATGCGGTTCACCAGGGTGTCGCCCTCGAACTTCACCTCGGCGCGGGTCTTGTAGTTGCCGTCGTCCTTGAAGAAGATGGTGCGCTCCTGGACGTAGCCTTCGGGCATGGCGGACTTGAAGAAGTCGTGCTGCTTCATGTGGTCGGGGTAGCGGCTGAAGCACTGCACGCCGTAGGTCAGGGTGGTCACGAGGGTGGGCCAGGGCACGGGCAGCTTGCCGGTGGTGCAGATGAACTTCAGGGTCAGCTTGCCGTAGGTGGCATCGCCCTCGCCCTCGCCGGACACGCTGAACTTGTGGCCGTTTACGTCGCCGTCCAGCTCGACCAGGATGGGCACCACCCCGGTGAACAGCTCCTCGCCCTTGCTCACCATCCCAGATCTTCCTGATCCGCTTGCGACAGGGGGCCCCTCCTCGTGTGGACCTGTAATTACGATTACTGGGTGTGGTCCCGCCATGGTAGGATCTAAGCTTCAACAGTACCGGAATGCCAAGCTGGAATTCGAGCTTCCATTATATACCCTCTAGAGTCTCCGCGGAAAGTCCCCAGCGGAAAGTCCCCAGCGGAAAGTCCCCAGCGGAAAGTCCCCAAGCGGAAAGTCCCCTAGCTTGGATCCAGACATGATAAGAGAGCTCAGGTACCTGTAACGCCATTTTGCAAGGCATGGAAAAATACCAAACCAAGAATAGAGAAGTTCAGATCAAGGGCGGGTACATGAAAATAGCTAACGTTGGGCCAAACAGGATATCTGCGGTGAGCAGTTTCGGCCCCGGCCCGGGGCCAAGAACAGATGGTCACCGCAGTTTCGGCCCCGGCCCGAGGCCAAGAACAGATGGTCCCCAGATATGGCCCAACCCTCAGCAGTTTCTTAAGACCCATCAGATGTTTCCAGGCTCCCCCAAGGACCTGAAATGACCCTGCGCCTTATTTGAATTAACCAATCAGCCTGCTTCTCGCTTCTGTTCGCGCGCTTCTGCTTCCCGAGCTCTATAAAAGAGCTCACAACCCCTCACTCGGCGCGCCAGTCCTCCGACAGACTGAGTCGGGATCCACCATGAGCGAGCTGATTAAGGAGAACATGCACATGAAGCTGTACATGGAGGGCACCGTGGACAACCATCACTTCAAGTGCACATCCGAGGGCGAAGGCAAGCCCTACGAGGGCACCCAGACCATGAGAATCAAGGTGGTCGAGGGCGGCCCTCTCCCCTTCGCCTTCGACATCCTGGCTACTAGCTTCCTCTACGGCAGCAAGACCTTCATCAACCACACCCAGGGCATCCCCGACTTTTTCAAGCAGTCCTTCCCTGAGGGCTTCACATGGGAGAGAGTCACCACATACGAAGACGGGGGCGTGCTGACCGCTACCCAGGACACCAGCCTCCAGGACGGCTGCCTCATCTACAACGTCAAGATCAGAGGGGTGAACTTTACATCCAACGGCCCTGTGATGCAGAAGAAAACACTCGGCTGGGAGGCCTTCACCGAGACGCTGTACCCCGCTGACGGCGGCCTGGAAGGCAGAAACGACATGGCCCTGAAGCTCGTGGGCGGGAGCCATCTGATCGCAAACATCAAGACCACATATAGATCCAAGAAACCCGCTAAGAACCTCAAGATGCCTGGCGTCTACTATGTGGACTACAGACTGGAAAGAATCAAGGAGGCCAACAACGAGACCTACGTCGAGCAGCACGAGGTGGCAGTGGCCAGATACTGCGACCTCCCTAGCAAACTGGGCCACAAGCTAAAT

SEQ ID NO: 81 - protein, 886 aa, synthetic construct
  REGION 1..886: note = Description of sequence: R2 3N PD-1EC CD28 delta Vmut3 P2A CD20 CD28EC CD28z
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKGGGGSGGGSTERILETSTRYPSPSPKPEGRFQGMFWALVVVAGVLFCYGLLVTVALCVIWTRAKFSRSAETAANLQDTFDALHMQTLAPRGGGGSGGGGSGGGGSTSMAGPVPRIIITKPEEEGSGTGRAKRGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 82 - protein, 954 aa, synthetic construct
  REGION 1..954: note = Description of sequence: R2 3N PD-1EC CD28 delta Vmut3 3x P2A CD20 CD28EC CD28z
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKGGGGSGGGSTERILETSTRYPSPSPKPEGRFQGMFWALVVVAGVLFCYGLLVTVALCVIWTRAKFSRSAETAANLQDTFDALHMQTLAPRGGGGSGGGGSGGGGSTSMAGPVPRIIITKPEEEGSTGGGGSGGGGSGGGGSMAGPVPRIIITKPEEEGSTGGGGSGGGGSGGGGSMAGPVPRIIITKPEEEGSGTGRAKRGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 83 - protein, 895 aa, synthetic construct
  REGION 1..895: note = Description of sequence: R2 3N PD-1EC CD28 delta DAMP P2A CD20 CD28EC CD28z
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKGGGGSGGGSTERILETSTRYPSPSPKPEGRFQGMFWALVVVAGVLFCYGLLVTVALCVIWTRAKFSRSAETAANLQDTFDALHMQTLAPRGGGGSGGGGSGGGGSTSITSFEEAKGLDRINERMPPRRDAMPGSGTGRAKRGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 84 - protein, 276 aa, synthetic construct
  REGION 1..276: note = Description of sequence: EGFP-DAMP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGGGSGGGSGSLITSFEEAKGLDRINERMPPRRDAMP

SEQ ID NO: 85 - protein, 1097 aa, synthetic construct
  REGION 1..1097: note = Description of sequence: Q2 RR12EE345L 2A iC9 CD19 CD8EC CD28z
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREQKLISEEDLLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 86 - protein, 1050 aa, synthetic construct
  REGION 1..1050: note = Description of sequence: FLAG-RR12EE345L P2A iC9 F2A CD19 CD8H CD8TM BBz
METDTLLLWVLLLWVPGSTGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREQKLISEEDLLESSTTTKPVLRTPSPVHPTGTSQPQRPEDCRPRGSVKGTGLDFACDIYIWAPLAGICVALLLSLIITLICYKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 87 - protein, 859 aa, synthetic construct
  REGION 1..859: note = Description of sequence: RR12EE345L linker EE12RR345L Thy1.1 P2A CD20 CD28H CD28TM BBz E2A
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGP

SEQ ID NO: 88 - protein, 1426 aa, synthetic construct
  REGION 1..1426: note = Description of sequence: FLAG-RR12EE345L P2A iC9 F2A 3xHA NFkBIAm E2A CD19 CD8H CD8TM BBz
METDTLLLWVLLLWVPGSTGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSGYPYDVPDYAGYPYDVPDYAGYPYDVPDYAGSGMFQPAGHGQDWAMEGPRDGLKKERLVDDRHDAGLDAMKDEEYEQMVKELREIRLQPQEAPLAAEPWKQQLTEDGDSFLHLAIIHEEKPLTMEVIGQVKGDLAFLNFQNNLQQTPLHLAVITNQPGIAEALLKAGCDPELRDFRGNTPLHLACEQGCLASVAVLTQTCTPQHLHSVLQATNYNGHTCLHLASIHGYLAIVEHLVTLGADVNAQEPCNGRTALHLAVDLQNPDLVSLLLKCGADVNRVTYQGYSPYQLTWGRPSTRIQQQLGQLTLENLQMLPEAEDEEAYDAEAEFAEDELPDDDCVFGGQRLTLGSGRAKRQCTNYALLKLAGDVESNPGPGSLTMASPLTRFLSLNLLLLGESIILGSGEAEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKREQKLISEEDLLESSTTTKPVLRTPSPVHPTGTSQPQRPEDCRPRGSVKGTGLDFACDIYIWAPLAGICVALLLSLIITLICYKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 89 - protein, 1207 aa, synthetic construct
  REGION 1..1207: note = Description of sequence: RR12EE345L linker EE12RR345L Thy1.1 P2A CD20 CD28H CD28TM BBz E2A 3xHA NFkBIAm
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLEYPYDVPDYAGYPYDVPDYAGYPYDVPDYAGSGMFQPAGHGQDWAMEGPRDGLKKERLVDDRHDAGLDAMKDEEYEQMVKELREIRLQPQEAPLAAEPWKQQLTEDGDSFLHLAIIHEEKPLTMEVIGQVKGDLAFLNFQNNLQQTPLHLAVITNQPGIAEALLKAGCDPELRDFRGNTPLHLACEQGCLASVAVLTQTCTPQHLHSVLQATNYNGHTCLHLASIHGYLAIVEHLVTLGADVNAQEPCNGRTALHLAVDLQNPDLVSLLLKCGADVNRVTYQGYSPYQLTWGRPSTRIQQQLGQLTLENLQMLPEAEDEEAYDAEAEFAEDELPDDDCVFGGQRLTL

SEQ ID NO: 90 - protein, 1388 aa, synthetic construct
  REGION 1..1388: note = Description of sequence: RR12EE345L linker EE12RR345L Thy1.1 CD20 CD28EC BBz 3xHA DHFR-DD NFkBIAm
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLEYPYDVPDYAGYPYDVPDYAGYPYDVPDYAGSGMFQPAGHGQDWAMEGPRDGLKKERLVDDRHDAGLDAMKDEEYEQMVKELREIRLQPQEAPLAAEPWKQQLTEDGDSFLHLAIIHEEKPLTMEVIGQVKGDLAFLNFQNNLQQTPLHLAVITNQPGIAEALLKAGCDPELRDFRGNTPLHLACEQGCLASVAVLTQTCTPQHLHSVLQATNYNGHTCLHLASIHGYLAIVEHLVTLGADVNAQEPCNGRTALHLAVDLQNPDLVSLLLKCGADVNRVTYQGYSPYQLTWGRPSTRIQQQLGQLTLENLQMLPEAEDEEAYDAEAEFAEDELPDDDCVFGGQRLTLGSGTRGSTGGGGSGGGGSGGGGSISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 91 - protein, 2623 aa, synthetic construct
  REGION 1..2623: note = Description of sequence: NFKBIAm NFAT SFFV BFP
CACACAAAAAACCAACACACAGATGTAATGAAAATAAAGATATTTTATTTAAGCTTTATTATAATGTCAGACGCTGGCCTCCAAACACACAGTCATCATCGGGCAGCTCATCCTCTGCGAACTCTGCCTCCGCGTCATAGGCCTCCTCATCCTCGGCCTCAGGTAGCATCTGGAGATTTTCCAAGGTCAGCTGGCCCAGCTGCTGCTGTATCCGGGTACTTGGGCGGCCCCAGGTAAGCTGGTAGGGGGAGTAGCCTTGGTAGGTTACCCTGTTGACATCAGCCCCACATTTCAACAAGAGCGAAACCAGGTCAGGATTCTGCAGGTCCACCGCAAGGTGGAGGGCTGTCCGGCCATTGCAGGGCTCCTGAGCGTTGACATCAGCACCCAAAGTCACCAAGTGCTCCACGATGGCCAGGTAGCCGTGGATAGAGGCTAGGTGCAGACACGTGTGGCCATTGTAGTTGGTGGCCTGTAGGACGGAGTGGAGATGCTGGGGTGTGCAGGTCTGCGTCAAGACTGCTACACTGGCCAGGCAGCCCTGCTCACAGGCAAGATGTAGAGGGGTATTTCCTCGAAAGTCTCGGAGTTCAGGATCACAGCCAGCTTTCAGAAGTGCCTCAGCAATTCCTGGCTGGTTGGTGATCACAGCCAAGTGGAGTGGAGTCTGCTGCAGGTTGTTCTGGAAGTTGAGGAAGGCCAGGTCTCCCTTCACCTGACCAATGACTTCCATTGTCAGCGGCTTCTCTTCGTGGATGATTGCCAAGTGCAGGAACGAGTCTCCGTCCTCCGTGAGCTGCTGCTTCCAGGGCTCGGCGGCCAGCGGCGCCTCCTGCGGCTGCAGGCGGATCTCCCGCAGCTCCTTCACCATTTGCTCGTACTCCTCGTCCTTCATGGCGTCAAGACCTGCGTCGTGGCGGTCGTCCACCAAGCGCTCCTTCTTGAGGCCATCCCGCGGGCCCTCCATTGCCCAGTCCTGGCCGTGCCCAGCTGGCTGAAACATTCCTGATCCAGCATAATCAGGAACGTCGTAAGGGTAGCCAGCGTAATCTGGAACATCGTATGGATAGCCAGCATAATCGGGAACATCATAGGGATACATGGTCCATGGACGCGTCAGGAGTTGAGGTTACTGTGAGTAGTGATTAAAGAGAGTGATAGGGAACTCTTGAACAAGAGATGCAATTTATACTGTTAATTCTGGAAAAATATTATGGGGGTGTCAAAATGTCCCGGGACCAATTGACGCCTTCTGTATGAAACAGTTTTTCCTCCTAATTGACGCCTTCTGTATGAAACAGTTTTTCCTCCTAATTGACGCCTTCTGTATGAAACAGTTTTTCCTCCTAATTCGATGGGACCAATTGACGCCTTCTGTATGAAACAGTTTTTCCTCCTAATTGACGCCTTCTGTATGAAACAGTTTTTCCTCCTAATTGACGCCTTCTGTATGAAACAGTTTTTCCTCCTAATTCGATATCAAGCTGGCGGCCGCAACTGGCCGGTACCTGTAACGCCATTTTGCAAGGCATGGAAAAATACCAAACCAAGAATAGAGAAGTTCAGATCAAGGGCGGGTACATGAAAATAGCTAACGTTGGGCCAAACAGGATATCTGCGGTGAGCAGTTTCGGCCCCGGCCCGGGGCCAAGAACAGATGGTCACCGCAGTTTCGGCCCCGGCCCGAGGCCAAGAACAGATGGTCCCCAGATATGGCCCAACCCTCAGCAGTTTCTTAAGACCCATCAGATGTTTCCAGGCTCCCCCAAGGACCTGAAATGACCCTGCGCCTTATTTGAATTAACCAATCAGCCTGCTTCTCGCTTCTGTTCGCGCGCTTCTGCTTCCCGAGCTCTATAAAAGAGCTCACAACCCCTCACTCGGCGCGCCAGTCCTCCGACAGACTGAGTCGGGATCCACCATGAGCGAGCTGATTAAGGAGAACATGCACATGAAGCTGTACATGGAGGGCACCGTGGACAACCATCACTTCAAGTGCACATCCGAGGGCGAAGGCAAGCCCTACGAGGGCACCCAGACCATGAGAATCAAGGTGGTCGAGGGCGGCCCTCTCCCCTTCGCCTTCGACATCCTGGCTACTAGCTTCCTCTACGGCAGCAAGACCTTCATCAACCACACCCAGGGCATCCCCGACTTTTTCAAGCAGTCCTTCCCTGAGGGCTTCACATGGGAGAGAGTCACCACATACGAAGACGGGGGCGTGCTGACCGCTACCCAGGACACCAGCCTCCAGGACGGCTGCCTCATCTACAACGTCAAGATCAGAGGGGTGAACTTTACATCCAACGGCCCTGTGATGCAGAAGAAAACACTCGGCTGGGAGGCCTTCACCGAGACGCTGTACCCCGCTGACGGCGGCCTGGAAGGCAGAAACGACATGGCCCTGAAGCTCGTGGGCGGGAGCCATCTGATCGCAAACATCAAGACCACATATAGATCCAAGAAACCCGCTAAGAACCTCAAGATGCCTGGCGTCTACTATGTGGACTACAGACTGGAAAGAATCAAGGAGGCCAACAACGAGACCTACGTCGAGCAGCACGAGGTGGCAGTGGCCAGATACTGCGACCTCCCTAGCAAACTGGGCCACAAGCTAAATTAA

SEQ ID NO: 92 - protein, 1203 aa, synthetic construct
  REGION 1..1203: note = Description of sequence: RR-EE Thy1.1 M1 VH-VL CD24 ST CD28EC CD28 1XX 3xHA NFkBIAm
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAEVKLQESGPGLVKPSLTLSLTCTVSGFSLNNYAVIWVRQPPGKGLEWMGVIWSDGKTNYNSALKSRLSISRDTSKSQVFLKMNNLQTEDTAMYFCARSSYYSSWFDSWGQGVMVTVSSGGGGSGGGGSGGGGSDIQLTQSPSFLSASVGDRVTLSCKASQNINKYLAWYQQKLGEAPKLLIYNANSLQTGIPSRFSGSGSGTDFTLTISSLQPEDVATYFCLQHNSWWTLGGGTKLELKRSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVFNALQKDKMAEAFSEIGTKGERRRGKGHDGLFQGLSTATKDTFDALHMQTLAPRGSGQCTNYALLKLAGDVESNPGPLEYPYDVPDYAGYPYDVPDYAGYPYDVPDYAGSGMFQPAGHGQDWAMEGPRDGLKKERLVDDRHDAGLDAMKDEEYEQMVKELREIRLQPQEAPLAAEPWKQQLTEDGDSFLHLAIIHEEKPLTMEVIGQVKGDLAFLNFQNNLQQTPLHLAVITNQPGIAEALLKAGCDPELRDFRGNTPLHLACEQGCLASVAVLTQTCTPQHLHSVLQATNYNGHTCLHLASIHGYLAIVEHLVTLGADVNAQEPCNGRTALHLAVDLQNPDLVSLLLKCGADVNRVTYQGYSPYQLTWGRPSTRIQQQLGQLTLENLQMLPEAEDEEAYDAEAEFAEDELPDDDCVFGGQRLTL

SEQ ID NO: 93 - protein, 842 aa, synthetic construct
  REGION 1..842: note = Description of sequence: R2 3N PD-1EC CD28 delta P2A CD20 CD28EC CD28z
METDTLLLWVLLLWVPGSTGCPYSNPSLCSGGGGSSGGGGSDCPYSNPSLCGGGGSGGGSLEIEAAFLRQENTALRTRVAELEQEVQRLENEVSQYETRYGPLGGGKGGGGSGGGGSGGGGSGGGSLEIEAAFLERENTALETRVAELRQRVQRLRNRVSQYRTRYGPLGGGKGGGGSGGGSTERILETSTRYPSPSPKPEGRFQGMFWALVVVAGVLFCYGLLVTVALCVIWTRAKFSRSAETAANLQDTFDALHMQTLAPRGSGATNFSLLKQAGDVEENPGPGSLTMASPLTRFLSLNLLLLGESIILGSGEAQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRGGGGSGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSSAWSHPQFEKGGGSGGGSGGSAWSHPQFEKGGGGSGGGSEARLIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGP